(19) United States
(12) Patent Application Publication
(10) Pub. No.: US 2026/0317746 A1
(43) Pub. Date: Oct. 15, 2026

(54) RECOMBINANT ACE2 POLYPEPTIDE AND USES THEREOF

(71) Applicant: Board of Supervisors of Louisiana State University and Agricultural and Mechanical College, Baton Rouge, LA (US)

(72) Inventors: Eric LAZARTIGUES, New Orleans, LA (US); Catalin FILIPEANU, Alexandria, VA (US)

(21) Appl. No.: 19/473,860

(22) PCT Filed: Apr. 10, 2024
(86) PCT No.: PCT/US2024/023899

§ 371 Date: Oct. 9, 2025

Related U.S. Application Data
(60) Provisional application No. 63/458,396, filed on Apr. 10, 2023.

(51) Int. Cl. C12N 9/48; A61K 38/00; A61P 9/00; C12N 15/86

(52) U.S. Cl. CPC C12N 9/485; A61P 9/00; C12N 15/86; A61K 38/00; C12N 2750/14143; C12Y 304/17023

(57) ABSTRACT

This invention is directed to recombinant ACE2 polypeptides and fragments thereof, compositions comprising the same, and methods for using the same to treat disease.

This application is an International Application, which claims the benefit of priority from U.S. provisional patent application No. 63/458,396, filed on Apr. 10, 2023, the entire contents of each which are incorporated herein by reference in their entireties.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### GOVERNMENT INTERESTS

This invention was made with government support under Grant No. HL150592 and P30GM106392 awarded by the National Institutes of Health, and Merit Award I01 BX004294 awarded by the United States Department of Veterans Affairs. The government has certain rights in the invention.

### FIELD OF THE INVENTION

This invention is directed to recombinant ACE2 polypeptides and fragments thereof, compositions comprising the same, and methods for using the same to treat disease.

### BACKGROUND OF THE INVENTION

Angiotensin converting enzyme 2 (ACE2) is a member of the renin-angiotensin system, primarily involved in the conversion of the vasoconstrictor octapeptide angiotensin (Ang)-II to the vasodilatory heptapeptide Ang-(1-7). Decreases in cellular ACE2 levels have been associated with cardiovascular diseases like hypertension and diabetes, while overexpression of ACE2 has been shown to have beneficial effects in preventing these diseases. ACE2 is a critical component of the compensatory renin-angiotensin system that is downregulated during the development of hypertension, possibly via ubiquitination. However, little is known about the mechanisms involved in ACE2 ubiquitination in neurogenic hypertension.

### SUMMARY OF THE INVENTION

An aspect of the invention is directed to a recombinant human Angiotensin Converting Enzyme type 2 (ACE2) polypeptide or fragment. In embodiments, the recombinant polypeptide comprises one or more substitutions selected from the group consisting of K769R, K770R, K771R, K773R, or K787R. In embodiments, the recombinant polypeptide or fragment thereof is resistant to protein degradation.

In embodiments, the recombinant polypeptide or fragment thereof described herein comprises an amino acid sequence according to:

MGVIVVGIVILIFTGIRDRRKKNKARSGENPYASIDISKGENNPG
FQNTDDVQTSF;
MGVIVVGIVILIFTGIRDRKRKNKARSGENPYASIDISKGENNPG
FQNTDDVQTSF;
MGVIVVGIVILIFTGIRDRKKRNKARSGENPYASIDISKGENNPG
FQNTDDVQTSE;
MGVIVVGIVILIFTGIRDRKKKNRARSGENPYASIDISKGENNPG
FQNTDDVQTSF;
MGVIVVGIVILIFTGIRDRKKKNKARSGENPYASIDISRGENNPG
FQNTDDVQTSF;
MGVIVVGIVILIFTGIRDRRRRNRARSGENPYASIDISRGENNPG
FQNTDDVQTSE,

or a sequence of at least 90% identity thereto.

In embodiments, the recombinant polypeptide or fragment thereof described herein consists of an amino acid sequence according to:

MGVIVVGIVILIFTGIRDRRKKNKARSGENPYASIDISKGENNPG
FQNTDDVQTSF;
MGVIVVGIVILIFTGIRDRKRKNKARSGENPYASIDISKGENNPG
FQNTDDVQTSF;
MGVIVVGIVILIFTGIRDRKKRNKARSGENPYASIDISKGENNPG
FQNTDDVQTSF;
MGVIVVGIVILIFTGIRDRKKKNRARSGENPYASIDISKGENNPG
FQNTDDVQTSF;
MGVIVVGIVILIFTGIRDRKKKNKARSGENPYASIDISRGENNPG
FQNTDDVQTSF;
MGVIVVGIVILIFTGIRDRRRRNRARSGENPYASIDISRGENNPG
FQNTDDVQTSE,

or a sequence of at least 90% identity thereto.

In embodiments, the recombinant polypeptide described herein comprises one or more post-translational modifications.

In embodiments, the recombinant polypeptide described herein exhibits at least one activity selected from the group consisting of resistance to ubiquitination, resistance to angiotensin II-induced degradation, enhanced enzymatic activity toward angiotensin formation, or increased cellular expression levels.

In embodiments, the recombinant polypeptide described herein is isolated from or secreted by a host cell. For example, the host cell is a mammalian cell, an insect cell, a yeast cell, or a bacterial cell.

Aspects of the invention are also drawn to a nucleic acid encoding the recombinant polypeptide or fragment described herein.

Further, aspects of the invention are drawn to a vector comprising the nucleic acid described herein.

Aspects of the invention are also drawn to a cell comprising the nucleic acid described herein. For example, the cell is a mammalian cell, bacterial cell, yeast cell, or an insect cell.

Further, aspects of the invention are drawn to a pharmaceutical composition comprising the recombinant polypeptide described herein, the nucleic acid described herein, or the cell described herein, and a pharmaceutically acceptable carrier.

In embodiments, the pharmaceutical composition described herein further comprises at least one additional active agent.

Aspects of the invention are also drawn to a method of treating a subject afflicted with a cardiovascular disease. In embodiments, the method comprises administering to the subject the recombinant polypeptide described herein, the nucleic acid described herein, the cell described herein, or the pharmaceutical composition described herein.

In embodiments, the cardiovascular disease comprises hypertension, type 2 diabetes, or heart failure.

Further, aspects of the invention are drawn to a method of treating a subject afflicted with a pulmonary disease. In embodiments, the method comprises administering to the subject the recombinant polypeptide described herein, the nucleic acid described herein, the cell described herein, or the pharmaceutical composition described herein.

In embodiments, the pulmonary disease comprises acute respiratory distress syndrome (ARDS) or COVID-19.

In embodiments, the administering is intracranially.

Other objects and advantages of this invention will become readily apparent from the ensuing description.

### BRIEF DESCRIPTION OF THE FIGURES

FIG. 1 provides data showing ubiquitination reduces ACE2 expression and activity. Panel A provides a representative blot showing the levels of ubiquitination of ACE2 in control HEK293T cells before and after Ang-II treatment. The first two lanes are from cells transfected with GFP-tagged ACE2, AT1R, HA-tagged ubiquitin, and pcDNA3.1, and the last two lanes are from cells transfected for 48 h, serum-starved for 24 h, and subsequently treated with (left) Ang-II (100 nM for 4 h) or Ang-II (100 nM) and losartan (1 μM) for 4 h. GFP was then immunoprecipitated from the cells, and immunoblotting was performed against HA, as described in the Methods section. Panel B provides a graph showing ACE2 activity in HEK293T cells transfected with pcDNA3.1 or HA-tagged ubiquitin (in duplicate from three separate transfections, n=6). Panel C provides a graph showing ACE2 activity (n=6), and Panel D shows expression (n=3) normalized to β-actin in HEK293T cells transfected with pcDNA3.1 and treated with PYR-41 (50 μM for 2 h), a cell-permeable inhibitor of E1 ligases, PR-619 (20 μM, 6 h), a broad-spectrum reversible inhibitor of cysteine-reactive deubiquitinases, or Ang-II (100 nM). Panel E provides a graph showing ACE2 expression in Neuro2A cells treated with the same drugs (n=5-6). Two-way analysis of variance (ANOVA) followed by Tukey's test for multiple comparisons. Statistical significance: *P<0.05, **P<0.01, ***P<0.001, and ****P<0.0001.

FIG. 2 provides data showing predicted ACE2-interacting Nedd4-2 is up-regulated in hypertension. Panel A provides bioinformatic analysis (UbiBrowser v1.0) showing E3 ubiquitin ligases predicted to interact with ACE2 based on E3 recognition motifs, network loops, and enriched gene ontology pairs. Panel B provides a schematic showing identification of four potential Nedd4-2 (also known as NEDD4L) recognition motifs (green) in the amino acid sequence of ACE2. Close-by lysine residues (K) are highlighted as potential ubiquitination sites. Panel C provides data showing changes in E3 ubiquitin ligase abundance identified by proteomic analysis from the hypothalamus of normotensive and Ang-II-infused mice from both sexes. Representative pictures and capillary western analysis of Nedd4-2 (130 kDa) and ACE2 (120 kDa) expression in the mouse (n=6/group) brain (Panel D), heart (Panel E), kidney (Panel F), and cardiac left ventricles (n=6/group) from African American donors (Panel G) normalized to GAPDH (~37 kDa). Two-way ANOVA followed by Tukey's test for multiple comparisons. Statistical significance: *P<0.05, **P<0.01, ***P<0.001, and ****P<0.0001.

FIG. 3 provides data showing ACE2 is ubiquitinated by Nedd4-2. Panel A provides a graph showing the impact of WT Nedd4-2 and its catalytically inactive mutant (C/S) transfection on ACE2 activity in HEK293T cells exposed to Ang-II (n=5). Panel B provides data showing modulation of ACE2 cellular levels by Nedd4-2 in HEK293T cells (n=4). Panel C provides images showing subcellular localization of ACE2 and Nedd4-2 in HAEC in the absence (control) or presence of Ang-II (100 nM for 4 h). Panel D provides representative pictures and capillary western analysis of Nedd4-2 and ACE2 expression in HAEC treated with Ang-II in the presence or absence of Nedd4-2 siRNA. Two-way ANOVA followed by Tukey's test for multiple comparisons, except for Panel B (one-way ANOVA followed by Bonferroni's test). Statistical significance: *P<0.05, **P<0.01, ***P<0.001, and ****P<0.0001. Scale bars are 10 μm.

FIG. 4 provides data showing ubiquitination-resistant ACE2. Panel A provides the amino acid sequence of the native ACE2 C-terminus and the six mutants (A) where lysine residues were replaced by arginine. The distal predicted Nedd4-2 recognition motif is highlighted in green. Panel B provides a graph showing ACE2 activity for WT and hACE2 mutants in the absence and presence of Ang-II (100 nM, 4 h). Panel C provides a representative western electrophoresis and quantified data showing the impact of Ang-II (100 nM, 4 h) on total cellular levels of ACE2 (n=6) in HEK293T cells transfected with WT ACE2 or ACE2-5R. All cells were transfected with AT1R plasmids. Panel D provides a schematic showing the hACE2-5R (Mutant 6) adeno-associated viral vector. Two-way ANOVA. Statistical significance: *P<0.05 and **P<0.01 vs. WT.

FIG. 5 provides data showing ACE2 is expressed on GABAergic neurons. Panel A provides a schematic showing the injection protocol and representative expression of AAV-ChR2-eYFP in the dorsal (dBNST) and ventral (vBNST) bed nucleus of the stria terminalis. Scale bar: 200 μm. Panel B provides immunohistochemistry pictures showing the co-localization of GABAergic neuron-targeted channelrhodopsin (ChR2) expression (green) with ACE2 (red). Photostimulations of ChR2 (10 Hz, 2 min, n=7) with blue LED on GABAergic cell bodies in the BNST (Panel C) and neuronal projections in the PVN (Panel D) lead to an immediate reduction in systolic BP and HR, while green LED is ineffective. Inserts show magnifications (x2) of boxed regions. Scale bars: 100 μm. Panel E provides data showing the changes in systolic BP and HR initiated by photoactivation of ChR2 in the BNST are prevented by GABAA receptor blockade by bicuculline (1 mM) in the PVN. Abbreviations: 3V, third ventricle; AC, anterior commissure.

FIG. 6 provides data showing ubiquitination-resistant ACE2-5R blunts neurogenic hypertension. Twenty-four-hour recording of BP and HR traces (Panel A) and average data at 4 weeks (Panel B) show that BNST expression of hACE2-5R blunted the development of Ang-II-mediated hypertension in mice during the active phase (grey box; n=5-6/group). The resting and active phases are indicated by the sun and moon symbols, respectively. Cumulative traces and the area under the curve (Panel C) show that PVN blockade of GABAA receptors with bicuculline (1 mM) mediates an enhanced systolic pressure response in Ang-II-infused mice expressing ACE2-5R in the BNST (n=4-6/group). Arrows indicate bicuculline bilateral injections. Capillary western analysis of Nedd4-2 (Panel D) (top band), ACE2 (Panel E), AT1R (Panel F), Mas1R (Panel G), and MDM2 (Panel H) expression in the BNST normalized to total proteins (n=7-10/group). Two-way ANOVA followed by Tukey's test for multiple comparisons. Statistical significance: *P<0.05, **P<0.01, ***P<0.001, and ****P<0.0001.

FIG. 7 provides a schematic of the role of ACE2 ubiquitination in neurogenic hypertension. ACE2 is expressed on GABAergic neurons within the BNST where elevated Ang-II levels lead to Nedd4-2 up-regulation and ACE2 ubiquitination. Ubiquitinated ACE2 is internalized and degraded, preventing the conversion of Ang-II to Ang-(1-7). This is associated with a reduction in the GABAergic inhibitory input to pre-sympathetic neurons in the PVN, contributing to the development of neurogenic hypertension. BNST-targeted expression of a ubiquitination-resistant ACE2-5R mutant preserved ACE2 expression on the cell surface, leading to Ang-II conversion to Ang-(1-7), a reduction of Nedd4-2 expression, enhanced GABAergic inhibitory tone to the PVN, and reduction in neurogenic hypertension.

FIG. 8 provides data showing capillary Western analysis of NEDD4-2 (~130 kDa) expression in cardiac left ventricles from Caucasian donors (n=5-8/group) normalized to GAPDH (~37 kDa).

FIG. 9 provides images showing HEK293T cells transfected with ACE2-GFP (green) and NEDD4-2 (red) in presence or absence of Ang-II. Scale bars are 10 μm.

FIG. 10 provides a schematic showing predicted Nedd4L substrates obtained from the UbiBrowser database. Green circles point to sodium channels and glutamate receptors involved in hypertension. Retrieved substrates of E3 ligase: Nedd4-2. 13 known substrates were retrieved (red). 2262 predicted substrates were retrieved with high confidence, with the top 20 predicted substrates shown (blue).

FIG. 11 provides a schematic showing the ACE2-mediated inhibitory signaling in the PVN microcircuits. Stimulation of ACE2 activity in peri-PVN regions converts Ang-II into Ang-(1-7) and enhances GABAergic signaling to the PVN region that inhibits neuronal activity of autonomic and neuroendocrine functions. Upon stimulation, ACE2 blunts the activity of AT1R-containing neurons, which also contain CRH, ADAM17, Glutamate, and decreases the excitatory input to pre-autonomic neurons that project to the brainstem cardiovascular nuclei (RVLM, NTS) and IML, leading to decreased sympathetic outflow. Similarly, ACE2 also directly influences oxytocin and vasopressin neurons in the PVN and modulates their neuroendocrine release in the circulation. Color coding, GABAergic neurons containing ACE2 (green), MasR (pink), AT2R (violet); gray-colored neurons represent pre-autonomic neurons containing ADAM17, glutamate and Sim1 along with GluR (blue), CRHR (violet). Abbreviations: A ACTH, adrenocorticotropic hormone; ADAM17, metallopeptidase domain 17; AVP, arginine vasopressin; A. Pituitary, anterior pituitary; CRH, corticotropic releasing hormone; CRHR, corticotropic releasing hormone receptor; GABAR, GABA receptor; Glu, glutamate; OXT, oxytocin; P. Pituitary, posterior pituitary; sim1, single-minded 1.

FIG. 12 provides a schematic showing ACE2 ubiquitination in neurogenic hypertension: a working model. ACE2-5R expression in the BNST blocks Ang II-mediated ACE2 ubiquitination by Nedd4-2, thus stabilizing ACE2 compensatory activity in GABAergic neurons and reinforcing the inhibitory tone to PVN glutamatergic neurons. Stimulation of GABA-A receptors in the PVN reduces the excitatory activity of these presympathetic neurons, leading to a reduction of sympathetic drive to end organs, ultimately resulting in a reduction of blood pressure. ACE2-5R, ubiquitination-resistant ACE2; AT1R, angiotensin II type 1 receptor; BNST, bed nucleus of the stria terminalis; GABA-AR, GABA-A receptor; PVN, paraventricular nucleus; RVLM, rostral ventrolateral medulla; Ub, ubiquitin.

FIG. 13 provides data showing UBR1 and ACE2 expression are differentially regulated by sex hormones in Ang-II-mediated hypertension. After 4 weeks of Ang-II infusion UBR1 (Panels A-C) and ACE2 expression was assessed in hypothalamus, heart, and kidney (Panels E-G) (n=6-8/group). Human cardiac left ventricle samples were obtained from the Medical College of Wisconsin and Duke University rejected donor databanks. Representative immunoassays for UBR1, ACE2 and GAPDH samples from Caucasian and Black donors (n=6-7/group) (Panels D, H). Data are shown as mean±SEM. Statistical significance: Two-way analysis of variance (ANOVA): ***P<0.0001, **P<0.01 and *P<0.05. Abbreviations: NT, normotensive; HT, hypertensive; 3, male; 9, female. Panel I provides a schematic showing the experimental protocol.

FIG. 14 provides data showing UBR1 contributes to Ang-II mediated ACE2 degradation. Human aortic endothelial cells were pre-treated with control or UBR1 siRNA for 6 hours before exposure to Ang-II (100 ng) for 4 hours. Panel A provides representative immunoassays for ACE2, UBR1 and B-Tubulin. Panel B provides data showing Ang-II induced a 2-fold increase in UBR1 levels that was blunted in cells pre-treated with UBR1 siRNA. Ang-II induced a dramatic (~80%) decrease in ACE2 levels. UBR1 knockdown did not affect basal expression of ACE2 but reversed the Ang-II mediated effect, resulting in an upregulation of ACE2 following Ang-II exposure. Panel C provides images showing Gfp-labeled ACE2 (Green) localizes at the plasma membrane in control cells and internalizes to the cytoplasm after Ang-II treatment. Texas red-labelled UBR1 is present throughout the cell and colocalizes with ACE2 mostly in the nucleus (DAPI, blue) following Ang-II treatment. Data are shown as mean±SEM (n=3, in triplicate). Statistical significance: One-way analysis of variance (ANOVA) followed by Bonferroni test for multiple comparisons, ***P<0.001 vs. Control siRNA: ###P<0.001 vs. Control siRNA+Ang-II and $$$P<0.001 vs. UBR1 siRNA.

FIG. 15 provides data showing UBR1 siRNA Infusion in Hypertensive Mice. Mice BP was recorded using radiotelemetry probes at baseline and after 1 week of Ang-II infusion (600 ng/kg/min) (Panel A). Experiment timeline of UBR1 siRNA infusion (Panel B). Weekly MAP recording in mice infused with either ICV (Panels C,D) or subcutaneous (Panels F,G) UBR1 siRNA. Bar charts showing average MAP per week in ICV (Panel E) and subcutaneous (Panel F) UBR1 siRNA groups. Unpaired student t test (E,H). n=4-5/group.

FIG. 16 provides a schematic showing that E3 ligases, DUB, and other agents regulating ACE2 ubiquitination. ACE2 ubiquitination leads to internalization and degradation in lysosomes and the proteasome. Several E3 ligases, miRNA, LPS, and vitamin C have been reported to promote ACE2 ubiquitination whereas DUBs, Apelin 13, long noncoding RNA, and MG132 oppose this post-translational modification. With the exception of Nedd4-2, the role of the reninangiotensin system in these processes has not been elucidated. ACE2, angiotensin-converting enzyme 2; AT1R, angiotensin II type 1 receptor; AT2R, angiotensin II type 2 receptor; DUB, deubiquitinase; LPS, lipopolysaccharides; MasR, Mas receptor; Ub, ubiquitin; Vit C, vitamin C.

FIG. 17 provides representative blot images showing ACE2 expression. Panel A provides a representative blot image showing the levels of ubiquitination of ACE2 in control HEK293T cells before and after Ang-II treatment. The first two lanes are from cells transfected with GFP-tagged ACE2, AT1R, HA-tagged ubiquitin, and pcDNA3.1, and the last two lanes are from cells transfected for 48 h, serum-starved for 24 h, and subsequently treated with (left) Ang-II (100 nM for 4 h) or Ang-II (100 nM) and losartan (1 μM) for 4 h. GFP was then immunoprecipitated from the cells, and immunoblotting was performed against HA, as described in herein. Panel B provides a representative blot image showing the input proteins for Panel A. Panel C provides a representative blot image showing ACE2 protein expression. Panel D provides a representative blot image showing beta actin loading control for Panel C. Panel E provides a representative blot image showing ACE2 protein expression. Data were normalized to total proteins.

FIG. 18 provides representative blot images showing Nedd4-2 expression, ACE2 expression, and GAPDH expression. Panel A provides a representative blot image showing Nedd4-2 expression in the brain, corresponding to FIG. 17. Panel B provides a representative blot image showing ACE2 expression in the brain, corresponding to FIG. 17. Panel C provides a representative blot image showing GAPDH expression in the brain, corresponding to FIG. 17. Panel D provides a representative blot image showing Nedd4-2 expression in the heart, corresponding to FIG. 17. Panel E provides a representative blot image showing ACE2 expression in the heart, corresponding to FIG. 17. Panel F provides a representative blot image showing GAPDH expression in the heart, corresponding to FIG. 17. Panel G provides a representative blot image showing Nedd4-2 expression in the kidney. Panel H provides a representative blot image showing ACE2 expression in the kidney. Panel I provides a representative blot image showing GAPDH expression in the kidney. Panel J provides a representative blot image showing Nedd4-2 expression in human cardiac left ventricle. Panel K provides a representative blot image showing ACE2 expression in human cardiac left ventricle. Panel L provides a representative blot image showing GAPDH expression in human cardiac left ventricle.

FIG. 19 provides representative blot images showing Nedd4-2 expression, ACE2 expression, and GAPDH expression. Panel A provides a representative blot image showing ACE2 expression in HEK293T cells. Panel B provides a representative blot image showing GAPDH expression in HEK293T cells. Panel C provides a representative blot image showing Nedd4-2 expression in human aortic endothelial cells. Panel D provides a representative blot image showing ACE2 expression in human aortic endothelial cells.

FIG. 20 provides representative blot images showing Nedd4-2 expression, ACE2 expression, and GAPDH expression. Panel A provides a representative blot image showing ACE2 expression in HEK293T cells. Panel B provides a representative blot image showing GAPDH expression in HEK293T cells.

FIG. 21 provides representative blot images showing Nedd4-2 expression, ACE2 expression, and GAPDH expression. Panel A provides a representative image showing Nedd4-2 expression in the mouse brain (bed nucleus of the stria terminalis). Panel B provides a representative blot image showing ACE2 expression in the mouse brain (bed nucleus of the stria terminalis). Panel C provides a representative blot image showing AT1 receptor expression in the mouse brain (bed nucleus of the stria terminalis). Panel D provides a representative blot image showing Mas receptor expression in the mouse brain (bed nucleus of the stria terminalis). Panel E provides a representative blot image showing MDM2 expression in the mouse brain (bed nucleus of the stria terminalis).

### DETAILED DESCRIPTION OF THE INVENTION

### Abbreviations and Definitions

Detailed descriptions of one or more embodiments are provided herein. It is to be understood, however, that the invention can be embodied in various forms. Therefore, specific details disclosed herein are not to be interpreted as limiting, but rather as a basis for the claims and as a representative basis for teaching one skilled in the art to employ the invention in any appropriate manner.

The singular forms “a”, “an” and “the” include plural reference unless the context clearly dictates otherwise. The use of the word “a” or “an” when used in conjunction with the term “comprising” in the claims and/or the specification can mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly, “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

The term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

The terms “ACE2” and “angiotensin-converting enzyme 2” can refer to a carboxypeptidase which is a member of the renin-angiotensin system (RAS), primarily involved in the conversion of the vasoconstrictor octapeptide angiotensin (Ang)-II to the vasodilatory heptapeptide Ang-(1-7).

### Recombinant ACE2 Polypeptides

Decreases in cellular ACE2 levels have been associated with cardiovascular diseases like hypertension and diabetes. Several post-translational mechanisms mediated by the Ang-II type 1 receptor (AT1R), involved in ACE2 down-regulation and their contribution to the development of neurogenic hypertension, including ACE2 shedding and internalization followed by degradation in lysosomes. Importantly, this latter mechanism may be associated with ubiquitination induced by elevated Ang-II levels.

```
An example amino acid sequence of Angiotensin Converting
Enzyme type 2 (ACE2) protein (isoform 1, uniprot id: Q9BYF1):
      1 msssswllls Ivavtaaqst ieeqaktfld kfnheaedlf yqsslaswny ntniteenvq
    61 nmnnagdkws aflkeqstla qmyplqeiqn ltvklqlqal qqngssvlse dkskrintil
  121 ntmstiystg kvcnpdnpqe clllepglne imansldyne rlwaweswrs evgkqlrply
  181 eeyvvlknem aranhyedyg dywrgdyevn gvdgydysrg qliedvehtf eeikplyehl
  241 hayvraklmn aypsyispig clpahllgdm wgrfwtnlys ltvpfgqkpn idvtdamvdq
  301 awdaqrifke aekffvsvgl pnmtqgfwen smltdpgnvq kavchptawd lgkgdfrilm
  361 ctkvtmddfl tahhemghiq ydmayaaqpf llrnganegf heavgeimsl saatpkhlks
  421 igllspdfqe dneteinfll kqaltivgtl pftymlekwr wmvfkgeipk dqwmkkwwem
  481 kreivgvvep vphdetycdp aslfhvsndy sfiryytrtl yqfqfqealc qaakhegplh
  541 kcdisnstea gqklfnmlrl gksepwtlal envvgaknmn vrpllnyfep lftwlkdqnk
  601 nsfvgwstdw spyadqsikv rislksalgd kayewndnem ylfrssvaya mrqyflkvkn
  661 qmilfgeedv rvanlkpris fnffvtapkn vsdiiprtev ekairmsrsr indafrlndn
  721 sleflgiqpt lgppnqppvs iwlivfgvvm gvivvgivil iftgirdrkk knkarsgenp
  781 yasidiskge nnpgfqntdd vqtsf
An example nucleotide sequence of ACE2 (human) (GenBank: AB046569.1):
      1 tttttagtct agggaaagtc attcagtgga tgtgatcttg gctcacaggg gacgatgtca
    61 agctcttcct ggctccttct cagccttgtt gctgtaactg ctgctcagtc caccattgag
  121 gaacaggcca agacattttt ggacaagttt aaccacgaag ccgaagacct gttctatcaa
  181 agttcacttg cttcttggaa ttataacacc aatattactg aagagaatgt ccaaaacatg
  241 aataacgctg gggacaaatg gtctgccttt ttaaaggaac agtccacact tgcccaaatg
  301 tatccactac aagaaattca gaatctcaca gtcaagcttc agctgcaggc tcttcagcaa
  361 aatgggtctt cagtgctctc agaagacaag agcaaacggt tgaacacaat tctaaataca
  421 atgagcacca tctacagtac tggaaaagtt tgtaacccag ataatccaca agaatgctta
  481 ttacttgaac caggtttgaa tgaaataatg gcaaacagtt tagactacaa tgagaggctc
  541 tgggcttggg aaagctggag atctgaggtc ggcaagcagc tgaggccatt atatgaagag
  601 tatgtggtct tgaaaaatga gatggcaaga gcaaatcatt atgaggacta tggggattat
  661 tggagaggag actatgaagt aaatggggta gatggctatg actacagccg cggccagttg
  721 attgaagatg tggaacatac ctttgaagag attaaaccat tatatgaaca tcttcatgcc
  781 tatgtgaggg caaagttgat gaatgcctat ccttcctata tcagtccaat tggatgcctc
  841 cctgctcatt tgcttggtga tatgtggggt agattttgga caaatctgta ctctttgaca
  901 gttccctttg gacagaaacc aaacatagat gttactgatg caatggtgga ccaggcctgg
  961 gatgcacaga gaatattcaa ggaggccgag aagttctttg tatctgttgg tcttcctaat
1021 atgactcaag gattctggga aaattccatg ctaacggacc caggaaatgt tcagaaagca
1081 gtctgccatc ccacagcttg ggacctgggg aaaggcgact tcaggatcct tatgtgcaca
1141 aaggtgacaa tggacgactt cctgacagct catcatgaga tggggcatat tcagtatgat
1201 atggcatatg ctgcacaacc ttttctgcta agaaatggag ctaatgaagg attccatgaa
1261 gctgttgggg aaatcatgtc actttctgca gccacaccta agcatttaaa atccattggt
1321 cttctgtcac ccgattttca agaagacaat gaaacagaaa taaacttcct gctcaaacaa
1381 gcactcacga ttgttgggac tctgccattt acttacatgt tagagaagtg gaggtggatg
1441 gtctttaaag gggaaattcc caaagaccag tggatgaaaa agtggtggga gatgaagcga
1501 gagatagttg gggtggtgga acctgtgccc catgatgaaa catactgtga ccccgcatct
1561 ctgttccatg tttctaatga ttactcattc attcgatatt acacaaggac cctttaccaa
1621 ttccagtttc aagaagcact ttgtcaagca gctaaacatg aaggccctct gcacaaatgt
1681 gacatctcaa actctacaga agctggacag aaactgttca atatgctgag gcttggaaaa
1741 tcagaaccct ggaccctagc attggaaaat gttgtaggag caaagaacat gaatgtaagg
1801 ccactgctca actactttga gcccttattt acctggctga aagaccagaa caagaattct
1861 tttgtgggat ggagtaccga ctggagtcca tatgcagacc aaagcatcaa agtgaggata
1921 agcctaaaat cagctcttgg agatagagca tatgaatgga acgacaatga aatgtacctg
1981 ttccgatcat ctgttgcata tgctatgagg cagtactttt taaaagtaaa aaatcagatg
2041 attctttttg gggaggagga tgtgcgagtg gctaatttga aaccaagaat ctcctttaat
2101 ttctttgtca ctgcacctaa aaatgtgtct gatatcattc ctagaactga agttgaaaag
2161 gccatcagga tgtcccggag ccgtatcaat gatgctttcc gtctgaatga caacagccta
2221 gagtttctgg ggatacagcc aacacttgga cctcctaacc agccccctgt ttccatatgg
2281 ctgattgttt ttggagttgt gatgggagtg atagtggttg gcattgtcat cctgatcttc
2341 actgggatca gagatcggaa gaagaaaaat aaagcaagaa gtggagaaaa tccttatgcc
2401 tccatcgata ttagcaaagg agaaaataat ccaggattcc aaaacactga tgatgttcag
2461 acctcctttt agaaaaatct atgtttttcc tottgaggtg attttgttgt atgtaaatgt
2521 taatttcatg gtatagaaaa tataagatga taaaaatatc attaaatgtc aaaactatga
2581 ctctgttcag aaaaaaaaa
```

As described herein, substitutions of lysine residues into arginine residues of positions 769, 770, 771, 773, and/or 787 of angiotensin-converting enzyme 2 (ACE2) prevent ubiquitin-associated protein degradation. Specifically, it was discovered that this mutant protein is resistant to ubiquitination and angiotensin II-induced degradation. Further, it was discovered that because the mutant protein cannot be degraded via ubiquitination, it displays an increased half-life and enhanced enzymatic activity toward formation of angiotensin (1-7), which is known to have beneficial effects on cardiovascular and pulmonary function. Accordingly, aspects of the invention are drawn to recombinant ACE2 polypeptides and fragments thereof that comprise one or more substitutions selected from the group comprising of K769R, K770R, K771R, K773R, or K787R.

The term “recombinant” can refer to genetic material (i.e., nucleic acids, the polypeptides they encode, and vectors and cells comprising such polynucleotides) that has been modified to alter its sequence or expression characteristics, such as by mutating the coding sequence to produce an altered polypeptide (i.e., a polypeptide comprising one or more amino acid substitutions). Recombinant nucleic acids, polypeptides, and cells based thereon, have been manipulated by man such that they are not identical to related nucleic acids, polypeptides, and cells found in nature. The term “recombinant polypeptide” can refer to a polypeptide that is produced by recombinant techniques, wherein DNA or RNA encoding the expressed protein is inserted into a suitable expression vector that is in turn used to transform a host cell to produce the polypeptide.

Routine methods for making recombinant nucleic acids can be used to construct expression vectors encoding the polypeptides of interest using appropriate transcriptional/translational control signals and the protein coding sequences. (See, for example, Sambrook et al., Molecular Cloning: A Laboratory Manual, 3d Ed. (Cold Spring Harbor Laboratory 2001)).

The terms “polypeptide,” “peptide” and “protein” can be used interchangeably herein and can refer to a polymer of amino acid residues. The terms can apply to amino acid polymers in which one or more amino acid residue is an artificial chemical mimetic of a corresponding naturally occurring amino acid, as well as to naturally occurring amino acid polymers and non-naturally occurring amino acid polymer.

The terms “amino acid” and “amino acid residue” can refer to natural amino acids, unnatural amino acids, and modified amino acids. Any reference to an amino acid, generally or specifically by name, includes reference to both the D and the L stereoisomers if their structure allows such stereoisomeric forms.

A polypeptide of the invention can be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides can have a defined three-dimensional structure, although they do not necessarily have such structure. Polypeptides with a defined three-dimensional structure are referred to as folded, and polypeptides which do not possess a defined three-dimensional structure, but rather can adopt a large number of different conformations, and are referred to as unfolded.

The invention encompasses ACE2 variants and fragments thereof that comprise one or more substitutions that, when compared to wild-type ACE2, are associated with increased activity and resistance to angiotensin (Ang)-II-mediated degradation. For example, one or more lysine residues can substituted with arginine in order to generate the recombinant ACE2 polypeptide and fragments thereof described herein. In embodiments, the one or more substitutions can be selected from a group consisting of K769R, K770R, K771R, K773R, K787R, K787R, or any combination thereof. For example, the one or more substitutions can be K769R/K770R/K771R/K773R/K787R/K787R.

In embodiments, the recombinant ACE2 polypeptide comprises an amino acid sequence according to:

MGVIVVGIVILIFTGIRDRRKKNKARSGENPYASIDISKGENNPG
FQNTDDVQTSF;
MGVIVVGIVILIFTGIRDRKRKNKARSGENPYASIDISKGENNPG
FQNTDDVQTSF;
MGVIVVGIVILIFTGIRDRKKRNKARSGENPYASIDISKGENNPG
FQNTDDVQTSF;
MGVIVVGIVILIFTGIRDRKKKNRARSGENPYASIDISKGENNPG
FQNTDDVQTSF;
MGVIVVGIVILIFTGIRDRKKKNKARSGENPYASIDISRGENNPG
FQNTDDVQTSF;
MGVIVVGIVILIFTGIRDRRRRNRARSGENPYASIDISRGENNPG
FQNTDDVQTSF.

In embodiments, the ACE2 recombinant polypeptide or fragment thereof described herein is resistant to protein degradation. The term “protein degradation” can refer to a natural process by which cells break down proteins that are damaged or no longer needed so they can be recycled or disposed of.

In embodiments, the recombinant polypeptide described herein is resistant or at least partially resistant to ubiquitination. The term “ubiquitination” can refer to the attachment of the protein ubiquitin to lysine residues of other molecules. Ubiquitination of a molecule, such as a peptide or protein, can act as a signal for its rapid cellular degradation, and for targeting to the proteasome complex. The process of ubiquitination involves a cascade of three enzymes, i.e., the ubiquitin activating enzyme (“E1”), the ubiquitin conjugating enzyme (“E2”), and the ubiquitin ligase (“E3”). In brief, target protein modification, through ubiquitination, occurs when ubiquitin is activated by E1, which then transfers the highly conserved ubiquitin molecules to an E2 intermediate. Finally, E3-mediated ligation permits the covalent attachment of ubiquitin to a substrate. Typically, ubiquitin-chain elongation occurs pursuant to the concerted action of E2 and E3, albeit through a process that is not completely understood. See Hochstrasser, M., “Lingering Mysteries of Ubiquitin-Chain Assembly.” Cell 124 (1): 27-34 (2006).

In embodiments, the recombinant ACE2 polypeptide or fragment thereof can be referred to as an “isolated polypeptide”. The term “isolated” can refer to a molecule of interest (such as, for example, a polynucleotide or a polypeptide) that is in an environment different from that in which the molecule was produced. For example, an “isolated” molecule is one which is substantially separated from the cellular components (e.g., membrane lipids, chromosomes, proteins) of the host cell from which it originated, or from the medium in which the host cell was cultured. The term does not require that the biomolecule has been separated from all other chemicals or molecules, although certain isolated biomolecules can be purified to near homogeneity. For example, an “isolated polypeptide” can refer to a polypeptide that is free from at least one contaminating polypeptide or other contaminants that are found in its natural environment. In embodiments, the isolated polypeptide is substantially free from any other contaminating polypeptides or other contaminants that are found in its natural environment which can interfere with its therapeutic, diagnostic, prophylactic or research use.

In embodiments, the recombinant ACE2 polypeptide or fragment thereof exhibits at least one functional and/or biological activity of wildtype ACE2. The phrase “biological activity” can refer to an in vivo activity, such as an activity within an organism or within a cell. Examples of such biological activities include, but are not limited to, resistance to ubiquitination, resistance to angiotensin II-induced degradation, enhanced enzymatic activity toward angiotensin formation, or increased cellular expression levels. In embodiments, the enzymatic activity of the ACE2 mutants described herein is enhanced and resistant to the effects of Ang-II treatment.

In embodiments, the polypeptide or fragment thereof can be isolated from or secreted by a host cell, such as a host cell which comprises a nucleic acid encoding for and expressing the polypeptide or fragment thereof. The phrase “host cell”, “host cell line,” and “host cell culture” can be used interchangeably and can refer to the cells into which exogenous nucleic acid can be introduced, including the progeny of such cells. Host cells include “transformants” and “transformed cells,” which include the primary transformed cell and the progeny derived therefrom without regard to the number of passages. The progeny does not need to be completely identical in nucleic acid content to a parent cell, but it can contain substitutions. Mutant progeny that have the same biological function or activity as screened or selected in the originally transformed cell are included in the invention. Host cells can include any eukaryotic cell or prokaryotic cell, including, but not limited, to a mammalian cell, avian cell, amphibian cell, plant cell, fish cell, insect cell, bacterial cell, yeast cell, whether in vitro or in vivo.

In embodiments, the host cell can be a mammalian cell. The phrase “mammalian cell” can refer to a cell of any mammal, including humans. The phrase can refer to cells in vivo, such as, for example, in an organism or in an organ of an organism. The phrase also can refer to cells in vitro, such as, for example, cells maintained in cell culture.

The recombinant ACE2 polypeptides described herein can be prepared using biological, chemical, and/or recombinant DNA techniques that are known in the art. Exemplary methods are described herein and in U.S. Pat. No. 6,872,700; WO 2007/139941; WO 2007/140284; WO 2008/082274; WO 2009/011544; and US Publication No. 2007/0238669, the disclosures of which are incorporated herein by reference in their entireties. Other methods for preparing the compounds are set forth herein.

### Fragments

In embodiments, the recombinant ACE2 polypeptide can comprise a fragment polypeptide (i.e., “fragment”, “variant”, or “derivative”), which can refer to a short amino acid sequence of a larger polypeptide. Protein fragments can be “free-standing,” or comprised within a larger polypeptide of which the fragment forms a part of. Representative examples of polypeptide fragments of the invention, include, for example, fragments comprising about 5 amino acids, about 10 amino acids, about 15 amino acids, about 20 amino acids, about 30 amino acids, about 40 amino acids, about 50 amino acids, about 60 amino acids, about 70 amino acids, about 80 amino acids, about 90 amino acids, about 100, about 200, and about 500 amino acids or more in length.

In embodiments, the fragments thereof retain at least some biological activity of its parent polypeptide. Polypeptides as described herein can include fragment, variant, or derivative molecules without limitation, so long as the polypeptide still serves its function. For example, such biological activities can comprise resistance to ubiquitination, resistance to angiotensin II-induced degradation, enhanced enzymatic activity toward angiotensin formation, or increased cellular expression levels. Polypeptide fragments, for example, can include proteolytic fragments, deletion fragments and fragments which more easily reach the site of action when delivered to an animal.

In embodiments, recombinant fragment polypeptides can comprise a fragment of an amino acid sequence according to

MGVIVVGIVILIFTGIRDRRKKNKARSGENPYASIDISKGENNPG
FQNTDDVQTSF;
MGVIVVGIVILIFTGIRDRKRKNKARSGENPYASIDISKGENNPG
FQNTDDVQTSF;
MGVIVVGIVILIFTGIRDRKKRNKARSGENPYASIDISKGENNPG
FQNTDDVQTSF;
MGVIVVGIVILIFTGIRDRKKKNRARSGENPYASIDISKGENNPG
FQNTDDVQTSF;
MGVIVVGIVILIFTGIRDRKKKNKARSGENPYASIDISRGENNPG
FQNTDDVQTSF;
MGVIVVGIVILIFTGIRDRRRRNRARSGENPYASIDISRGENNPG
FQNTDDVQTSF,

or a sequence that is at least 60%, at least 65%, at least 70%, at least 75%, at least 80%, at least 85%, at least 90%, at least 95%, or greater than 95% identical thereto. In some embodiments, the fragment can be about 5 amino acids of an amino acid sequence described herein; from about 5 amino acids to about 10 amino acids of an amino acid sequence described herein; from about 10 amino acids to about 20 amino acids of an amino acid sequence described herein; from about 20 amino acids to about 30 amino acids of an amino acid sequence described herein; from about 30 amino acids to about 40 amino acids of an amino acid sequence described herein; from about 40 amino acids to about 50 amino acids of an amino acid sequence described herein; from about 50 amino acids to about 60 amino acids of an amino acid sequence described herein; from about 60 amino acids to about 70 amino acids of an amino acid sequence described herein; from about 70 amino acids to about 80 amino acids of an amino acid sequence described herein; from about 80 amino acids to about 90 amino acids of an amino acid sequence described herein; from about 90 amino acids to about 100 amino acids of an amino acid sequence described herein; from about 100 amino acids to about 110 amino acids of an amino acid sequence described herein; from about 110 amino acids to about 120 amino acids of an amino acid sequence described herein; from about 120 amino acids to about 130 amino acids of an amino acid sequence described herein; from about 130 amino acids to about 135 amino acids of an amino acid sequence described herein.

### Variants

Embodiments are also drawn to variants of the ACE2 polypeptide or fragment which substantially retains the property of being resistant to protein degradation. A variant sequence can be obtained by addition, deletion, substitution, modification, replacement and/or variation of at least one residue present in the amino acid sequences described herein.

The terms “amino acid substitution” or “substitution” can refer to replacing at least one amino acid residue present in a previously determined amino acid sequence with another different “substitution” amino acid residue. For example, one or more substitutions of lysine residues into arginine residues, such as at one or more of amino acid positions 769, 770, 771, 773, or 787 of angiotensin-converting enzyme 2 (ACE2), prevent ubiquitin-associated protein degradation. Without wishing to be bound by theory, substituting lysine with other amino acids will render the same effect. For example, like lysine, the artisan will recognize that histidine is an amino acid that closely resembles the polarity of arginine. Without wishing to be bound by theory, His→Arg substitutions, just like Lys→Arg substitutions, will preserve interactions of ACE2 which are dependent on polarity.

The term “deletion” can refer to a change in a reference amino acid sequence that results in the absence of one or more amino acid residues. A deletion removes at least 1, 2, 3, 4, 5, 10, 20, 50, 100, or 200 amino acids residues or a range of amino acid residues bounded by any of these values (e.g., a deletion of 5-10 amino acids). A deletion may include an internal deletion or a terminal deletion (e.g., an N-terminal truncation or a C-terminal truncation of a reference polypeptide). A “variant” of a reference polypeptide sequence may include a deletion relative to the reference polypeptide sequence.

The words “insertion” and “addition” refer to changes in an amino acid sequence resulting in the addition of one or more amino acid residues. An insertion or addition may refer to 1, 2, 3, 4, 5, 10, 20, 30, 40, 50, 60, 70, 80, 90, 100, 150, or 200 amino acid residues or a range of amino acid residues bounded by any of these values (e.g., an insertion or addition of 5-10 amino acids).

The terms “identical” or “percent identity,” in the context of two or more nucleic acids or polypeptide sequences, can refer to two or more sequences or subsequences that are the same or have a specified percentage of amino acid residues or nucleotides that are the same (i.e., about 60%, 65%, 70%, 75%, 80%, 85%, 90%, 91%, 92%, 93%, 94%, 95%, 96%, 97%, 98%, 99%, or higher identity over a specified region, when compared and aligned for maximum correspondence over a specified region). Percent identity can be measured by the eye or, more usually, with the aid of readily available sequence comparison programs. These commercially available computer programs can calculate precent identity between two or more sequences. For example, using a BLAST or BLAST 2.0 sequence comparison algorithms with default parameters set, or by manual alignment and visual inspection. Such sequences are then said to be “substantially identical.” The definition can also include sequences that have deletions and/or additions, as well as those that have substitutions. Algorithms can account for gaps and the like.

Percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 100, at least 150, at least 200, at least 250, at least 300, at least 350, at least 400, at least 450, at least 500, at least 550, at least 600, at least 650, or at least 700 contiguous amino acid residues; or a fragment of no more than 15, 20, 30, 40, 50, 100, 150, 200, 250, 300, 350, 400, 450, 500, 550, 600, 650, or 700 amino acid residues; or over a range bounded by any of these values (e.g., a range of 500-600 amino acid residues) Such lengths are exemplary only, and it is understood that any fragment length supported by the sequences shown herein, in the tables, figures or Sequence Listing, may be used to describe a length over which percentage identity may be measured.

For sequence comparison, for example, one sequence acts as a reference sequence, to which test sequences are compared. When using a sequence comparison algorithm, test and reference sequences are entered into a computer, subsequence coordinates are selected, if necessary, and sequence algorithm program parameters are selected. Default program parameters can be used, or alternative parameters can be selected. The sequence comparison algorithm then calculates the percent sequence identities for the test sequences relative to the reference sequence, based on the program parameters.

### Protein Modifications

In embodiments, the polypeptide can comprise one or more modifications. The term “modification” or “protein modification” can refer to one or more post-translational modifications or amino acid mutations (e.g., substitutions, deletions, and/or insertions as described herein), as is well understood in the art.

The term “amino acid mutation” can refer to one or more mutations of amino acid positions on a fragment of polypeptide and variants thereof, wherein the variant can be obtained by substituting, inserting or deleting amino acids at one or some sites on the polypeptide.

The polypeptide fragment of ACE2 may be modified to increase half-life in plasma and/or to enhance delivery to a target (e.g., the kidney, the lungs, the heart, etc.). Strategies to improve plasma half-life of peptide and protein drugs are known in the art. (See Werle et al., “Strategies to improve plasma half life time of peptide and protein drugs,” Amino Acids 2006 June; 30 (4): 351-67).

In embodiments, the recombinant ACE2 polypeptide can comprise one or more post-translational modifications. The term “post-translational modification” can refer to a modification that increases the functional diversity of the proteome by the covalent addition of functional groups or proteins, proteolytic cleavage of regulatory subunits, or degradation of entire proteins. Non-limiting examples of post-translational modifications include phosphorylation, glycosylation, ubiquitination, nitrosylation, methylation, acetylation, acylation, SUMOylation, deamination, lipidation and proteolysis, and influence almost all aspects of normal cell biology and pathogenesis.

### Nucleic Acids

Aspects of the invention are also drawn to nucleic acids encoding a recombinant ACE2 polypeptide or fragment thereof. In embodiments, the nucleic acid comprises a nucleotide sequence encoding the recombinant polypeptide or a fragment thereof described herein, or a sequence that is at least 75%, at least 80%, at least 85%, at least 90%, at least 95%, or greater than 95% identical thereto.

In embodiments, the nucleic acid comprises a “nucleic acid fragment”, which can refer to a portion of a larger nucleic acid molecule.

Polynucleotides of the invention may comprise DNA or RNA, preferably DNA. They may be single-stranded or double-stranded. It will be understood by a skilled person that numerous different polynucleotides can encode the same polypeptide as a result of the degeneracy of the genetic code. In addition, it is to be understood that skilled persons may, using routine techniques, make nucleotide substitutions that do not affect the polypeptide sequence encoded by the polynucleotides of the invention to reflect the codon usage of any particular host organism in which the polypeptides of the invention are to be expressed. The nucleotide sequences of the invention disclosed herein may comprise or lack stop codons at their 3′ end, for example depending on their position in a bicistronic vector. Thus, the present disclosure encompasses the SEQ ID NOs disclosed herein with the stop codons present or absent.

The polynucleotides may be modified by any method available in the art. Such modifications may be carried out in order to enhance the in vivo activity or lifespan of the polynucleotides of the invention.

Polynucleotides such as DNA polynucleotides may be produced recombinantly, synthetically or by any means available to those of skill in the art. They may also be cloned by standard techniques. Longer polynucleotides will generally be produced using recombinant means, for example using polymerase chain reaction (PCR) cloning techniques. This will involve making a pair of primers (e.g. of about 15 to 30 nucleotides) flanking the target sequence which it is desired to clone, bringing the primers into contact with mRNA or cDNA obtained from an animal or human cell, performing a polymerase chain reaction under conditions which bring about amplification of the desired region, isolating the amplified fragment (e.g. by purifying the reaction mixture with an agarose gel) and recovering the amplified DNA. The primers may be designed to contain suitable restriction enzyme recognition sites so that the amplified DNA can be cloned into a suitable vector.

A vector is a tool that allows or facilitates the transfer of an entity from one environment to another. In one aspect, the invention provides a vector comprising a polynucleotide of the invention. In preferred embodiments, the vector is a viral vector. In some embodiments, the vector is an adeno-associated viral (AAV) vector, retroviral vector, lentiviral vector or adenoviral vector, preferably an AAV vector.

The polynucleotide or vector of the invention may also include elements allowing for the expression of the nucleotide sequence encoding the protein of the invention in vitro or in vivo. These may be referred to as expression control sequences. Thus, the polynucleotide or vector typically comprises expression control sequences (e.g. comprising a promoter sequence) operably linked to the nucleotide sequence encoding the protein of the invention.

Any suitable promoter may be used, the selection of which may be readily made by the skilled person. The promoter sequence may be constitutively active (i.e. operational in any host cell background), or alternatively may be active only in a specific host cell environment, thus allowing for targeted expression of the transgene in a particular cell type (e.g. a tissue-specific promoter). The promoter may show inducible expression in response to presence of another factor, for example a factor present in a host cell. In any event, where the vector is administered for therapy, it is preferred that the promoter should be functional in the target cell background.

The polynucleotide or vector of the invention may also comprise one or more additional regulatory sequences which may act pre- or post-transcriptionally. The regulatory sequence may be part of the native transgene locus or may be a heterologous regulatory sequence. The polynucleotide or vector of the invention may comprise portions of the 5′UTR or 3′-UTR from the native transgene transcript.

Regulatory sequences are any sequences which facilitate expression of the transgene, i.e., act to increase expression of a transcript, improve nuclear export of mRNA or enhance its stability. Such regulatory sequences include for example enhancer elements, post-transcriptional regulatory elements and polyadenylation sites.

Suitable enhancers include the WPRE regulatory element. Suitable poly-A signals include the Bovine Growth Hormone poly-A signal.

Additional regulatory sequences may be readily selected by the skilled person.

Aspects of the invention are further drawn to a cell comprising the nucleic acids described herein. As described herein, the cell can be referred to as a host cell. For example, the phrase “host cell”, “host cell line,” and “host cell culture” can be used interchangeably and can refer to the cells into which exogenous nucleic acid can be introduced, including the progeny of such cells. Host cells include “transformants” and “transformed cells,” which include the primary transformed cell and the progeny derived therefrom without regard to the number of passages. The progeny does not need to be completely identical in nucleic acid content to a parent cell, but it can contain mutations. Mutant progeny that have the same biological function or activity as screened or selected in the originally transformed cell are included in the invention. Host cells can include any eukaryotic cell or prokaryotic cell, including, but not limited, to a mammalian cell, an insect cell, bacterial cell, yeast cell, whether in vitro or in vivo.

In embodiments, the host cell can be a mammalian cell. The phrase “mammalian cell” can refer to a cell of any mammal, including humans. The phrase can refer to cells in vivo, such as, for example, in an organism or in an organ of an organism. The phrase also can refer to cells in vitro, such as, for example, cells maintained in cell culture.

The engineered polypeptides described herein can be designed at the amino acid level. These sequences can then be back translated using a variety of software products known in the art such that the nucleotide sequence is optimized for expression, such as in a given host (for example, based protein expression, codon optimization, restriction site content). For example, the nucleotide sequence can be optimized for E. coli based protein expression and for restriction site content. Based on the nucleotide sequence of interest, overlapping oligonucleotides can be provided for multistep PCR, as known in the art. These oligonucleotides can be used in multiple PCR reactions under conditions well known in the art to build the cDNA encoding the protein of interest. The skilled artisan will recognize that there are multiple ways to generate genes of interest, in addition to synthesizing an entire cDNA of interest.

The recombinant ACE2 polypeptides and fragments thereof described herein can be prepared using recombinant DNA techniques using methods known in the art, such as Sambrook et al., 1989, MOLECULAR CLONING: A LABORATORY MANUAL, 2d Ed., Cold Spring Harbor. Non-peptide compounds can be prepared by art-known methods. For example, phosphate-containing amino acids and peptides containing such amino acids, can be prepared using methods known in the art, such as described in Bartlett et al, 1986, Biorg. Chem. 14:356-377.

The engineered polypeptides can alternatively be produced by recombinant techniques well known in the art. See, e.g., Sambrook et al., 1989 (Id.). These engineered polypeptides produced by recombinant technologies can be expressed from a polynucleotide. One skilled in the art will appreciate that the polynucleotides, including DNA and RNA, that encode such engineered polypeptides can be obtained from the wild-type cDNA, taking into consideration the degeneracy of codon usage, and can be further engineered to incorporate the indicated substitutions. These polynucleotide sequences can incorporate codons facilitating transcription and translation of mRNA in microbial hosts. Such manufacturing sequences can readily be constructed according to the methods well known in the art. See, e.g., WO 83/04053, incorporated herein by reference in its entirety. The polynucleotides described herein can also optionally encode an N-terminal methionyl residue. Non-peptide compounds useful in the invention can be prepared by art-known methods. For example, phosphate-containing amino acids and peptides containing such amino acids can be prepared using methods known in the art. See, e.g., Bartlett and Landen, 1986, Bioorg. Chem. 14:356-77.

A variety of expression vector/host systems can be utilized to contain and express an engineered polypeptide coding sequence. These include but are not limited to microorganisms such as bacteria transformed with recombinant bacteriophage, plasmid or cosmid DNA expression vectors; yeast transformed with yeast expression vectors; insect cell systems infected with virus expression vectors (e.g., baculovirus); plant cell systems transfected with virus expression vectors (e.g., cauliflower mosaic virus, CaMV; tobacco mosaic virus, TMV) or transformed with bacterial expression vectors (e.g., Ti or pBR322 plasmid); or animal cell systems. Mammalian cells that are useful in recombinant protein productions include but are not limited to VERO cells, HeLa cells, Chinese hamster ovary (CHO) cell lines, COS cells (such as COS-7), WI 38, BHK, HepG2, 3T3, RIN, MDCK, A549, PC12, K562 and 293 cells. Exemplary protocols for the recombinant expression of the protein are described herein and/or are known in the art.

As such, polynucleotide sequences are useful in generating new and useful viral and plasmid DNA vectors, new and useful transformed and transfected prokaryotic and eukaryotic host cells (including bacterial, yeast, and mammalian cells grown in culture), and new and useful methods for cultured growth of such host cells that can express the engineered polypeptides. The polynucleotide sequences encoding engineered polypeptides herein can be useful for gene therapy in instances where underproduction of engineered polypeptides can be alleviated, or the need for increased levels of such can be met.

This invention also provides for processes for recombinant DNA production of the engineered polypeptides. Provided is a process for producing the engineered polypeptides from a host cell containing nucleic acids encoding the engineered polypeptide comprising: (a) culturing the host cell containing polynucleotides encoding the engineered polypeptide under conditions facilitating the expression of the DNA molecule; and (b) obtaining the engineered polypeptide.

Host cells can be prokaryotic or eukaryotic and include bacteria, mammalian cells (such as Chinese Hamster Ovary (CHO) cells, monkey cells, baby hamster kidney cells, cancer cells or other cells), yeast cells, and insect cells.

Mammalian host systems for the expression of the recombinant protein also are well known to those of skill in the art. Host cell strains can be chosen for their ability to process the expressed protein or produce certain post-translation modifications that will be useful in providing protein activity. Such modifications of the polypeptide include, but are not limited to, acetylation, carboxylation, glycosylation, phosphorylation, lipidation and acylation. Post-translational processing, which cleaves a “prepro” form of the protein, can also be important for correct insertion, folding and/or function. Different host cells, such as CHO, HeLa, MDCK, 293, W138, and the like, have specific cellular machinery and characteristic mechanisms for such post-translational activities, and can be chosen to ensure the correct modification and processing of the introduced foreign protein.

Alternatively, a yeast system can be employed to generate the engineered polypeptides of the invention. The coding region of the engineered polypeptides DNA is amplified by PCR. A DNA encoding the yeast pre-pro-alpha leader sequence is amplified from yeast genomic DNA in a PCR reaction using one primer containing nucleotides 1-20 of the alpha mating factor gene and another primer complementary to nucleotides 255-235 of this gene (Kurjan and Herskowitz, 1982, Cell, 30:933-43). The pre-pro-alpha leader coding sequence and engineered polypeptide coding sequence fragments are ligated into a plasmid containing the yeast alcohol dehydrogenase (ADH2) promoter, such that the promoter directs expression of a fusion protein consisting of the pre-pro-alpha factor fused to the mature engineered polypeptide. As taught by Rose and Broach, Meth. Enz. 185:234-79, Goeddel ed., Academic Press, Inc., San Diego, Calif. (1990), the vector further includes an ADH2 transcription terminator downstream of the cloning site, the yeast “2-micron” replication origin, the yeast leu-2d gene, the yeast REP1 and REP2 genes, the E. coli beta-lactamase gene, and an E. coli origin of replication. The beta-lactamase and leu-2d genes provide for selection in bacteria and yeast, respectively. The leu-2d gene also facilitates increased copy number of the plasmid in yeast to induce higher levels of expression. The REP1 and REP2 genes encode proteins involved in regulation of the plasmid copy number.

The DNA construct described in the preceding paragraph is transformed into yeast cells using a known method, e.g., lithium acetate treatment (Steams et al., 1990, Meth. Enz. 185:280-297). The ADH2 promoter is induced upon exhaustion of glucose in the growth media (Price et al., 1987, Gene 55:287). The pre-pro-alpha sequence effects secretion of the fusion protein from the cells. Concomitantly, the yeast KEX2 protein cleaves the pre-pro sequence from the mature engineered polypeptides (Bitter et al., 1984, Proc. Natl. Acad. Sci. USA 81:5330-5334).

Engineered polypeptides of the invention can also be recombinantly expressed in yeast, e.g., Pichia, using a commercially available expression system, e.g., the Pichia Expression System (Invitrogen, San Diego, Calif.), following the manufacturer's instructions. This system also relies on the pre-pro-alpha sequence to direct secretion, but transcription of the insert is driven by the alcohol oxidase (AOX1) promoter upon induction by methanol. The secreted engineered polypeptide is purified from the yeast growth medium by, e.g., the methods used to purify said engineered polypeptide from bacterial and mammalian cell supernatants.

Alternatively, the DNA encoding an engineered polypeptide can be cloned into a baculovirus expression vector, e.g. pVL1393 (PharMingen, San Diego, Calif.). This engineered-polypeptide-encoding vector is then used according to the manufacturer's directions (PharMingen) or known techniques to infect Spodoptera frugiperda cells, grown for example in sF9 protein-free media, and to produce recombinant protein. The protein is purified and concentrated from the media using methods known in the art, e.g. a heparin-Sepharose column (Pharmacia, Piscataway, N.J.) and sequential molecular sizing columns (Amicon, Beverly, Mass.), and resuspended in appropriate solution, e.g. PBS. SDS-PAGE analysis can be used to characterize the protein, for example by showing a single band that confirms the size of the engineered polypeptide, as can full amino acid amino acid sequence analysis, e.g. Edman sequencing on a Proton 2090 Peptide Sequencer, or confirmation of its N-terminal sequence.

For example, the DNA sequence encoding the predicted mature engineered polypeptide can be cloned into a plasmid containing a promoter and, optionally, a leader sequence (see, e.g., Better et al., 1988, Science 240:1041-1043). The sequence of this construct can be confirmed by automated sequencing. The plasmid can then be transformed into E. coli, strain MC1061, using standard procedures employing CaCl2 incubation and heat shock treatment of the bacteria (Sambrook et al., Id.). The transformed bacteria are grown in LB medium supplemented with carbenicillin, and production of the expressed protein is induced by growth in a suitable medium. The leader sequence can affect secretion of the mature engineered polypeptide and be cleaved during secretion. The secreted recombinant engineered polypeptide can be purified from the bacterial culture media by the method described herein.

In another embodiment, the engineered polypeptides can be expressed in an insect system. Insect systems for protein expression are well known to those of skill in the art. In one such system, Autographa californica nuclear polyhedrosis virus (AcNPV) is used as a vector to express foreign genes in Spodoptera frugiperda cells or in Trichoplusia larvae. The engineered polypeptide coding sequence is cloned into a nonessential region of the virus, such as the polyhedrin gene, and placed under control of the polyhedrin promoter. Successful insertion of an engineered polypeptide will render the polyhedrin gene inactive and produce recombinant virus lacking coat protein coat. The recombinant viruses are then used to infect S. frugiperda cells or Trichoplusia larvae in which engineered polypeptide of the invention is expressed (Smith et al., 1983, J. Virol. 46:584; Engelhard et al., 1994, Proc. Natl. Acad. Sci. USA 91:3224-3227).

In another example, the DNA sequence encoding the engineered polypeptides can be amplified by PCR and cloned into an appropriate vector, for example, pGEX-3X (Pharmacia, Piscataway, N.J.). The pGEX vector is designed to produce a fusion protein comprising glutathione-S-transferase (GST), encoded by the vector, and a protein encoded by a DNA fragment inserted into the vector's cloning site. The primers for the PCR can be generated to include, for example, an appropriate cleavage site. The recombinant fusion protein can then be cleaved from the GST portion of the fusion protein. The pGEX-3X/engineered polypeptide construct is transformed into E. coli XL-1 Blue cells (Stratagene, La Jolla, Calif.), and individual transformants are isolated and grown at 37° C. in LB medium (supplemented with carbenicillin) to an optical density at wavelength 600 nm of 0.4, followed by further incubation for 4 hours in the presence of 0.5 mM Isopropyl beta-D-thiogalactopyranoside (Sigma Chemical Co., St. Louis, Mo.). Plasmid DNA from individual transformants is purified and partially sequenced using an automated sequencer to confirm the presence of the engineered polypeptide-encoding gene insert in the proper orientation.

In embodiments, viral delivery systems can be used to transduce cells. For example, lentiviral infection (i.e., lentiviral transduction) can be used to introduce an exogenous polynucleotide into the genome of a cell. The use of lentiviral vectors permits stable expression of the polynucleotide of interest. For example, lentiviral transduction can be performed by incubating cells to be transduced with a lentiviral vector carrying at least one polynucleotide encoding a polypeptide of interest.

In embodiments, the transformed cells are used for long-term, high-yield protein production and as such stable expression is desirable. Once such cells are transformed with vectors that contain selectable markers along with the expression cassette, the cells can be allowed to grow for 1-2 days in an enriched media before they are switched to selective media. The selectable marker can be designed to confer resistance to selection, and its presence allows growth and recovery of cells that successfully express the introduced sequences. Resistant clumps of stably transformed cells can be proliferated using tissue culture techniques appropriate to the cell.

A number of selection systems can be used to recover the cells that have been transformed for recombinant protein production. Such selection systems include, but are not limited to, HSV thymidine kinase, hypoxanthine-guanine phosphoribosyltransferase and adenine phosphoribosyltransferase genes, in tk-, hgprt- or aprt-cells, respectively. Also, anti-metabolite resistance can be used as the basis of selection for dhfr, that confers resistance to methotrexate; gpt, that confers resistance to mycophenolic acid; neo, that confers resistance to the aminoglycoside, G418; also, that confers resistance to chlorsulfuron; hygro, that confers resistance to hygromycin; or PAC, that confers resistance to puromycin. Additional selectable genes that can be useful include trpB, which allows cells to utilize indole in place of tryptophan, or hisD, which allows cells to utilize histinol in place of histidine. Markers that give a visual indication for identification of transformants include anthocyanins, beta-glucuronidase and its substrate, GUS, and luciferase and its substrate, luciferin.

The engineered polypeptides of the invention can be produced using a combination of both automated peptide synthesis and recombinant techniques. Engineered polypeptides can be made synthetically or recombinantly and then, in embodiments, ligated together using methods known in the art, such as “native chemical ligation” and known variations thereof in which an amide bond is formed joining the parent compounds. See for example U.S. Pat. No. 6,326,468. In other embodiments, for example, an engineered polypeptide of the invention can contain a combination of modifications including deletion, substitution, insertion and derivatization by PEGylation (or other moiety, e.g. polymer, fatty acyl chain, C-terminal amidation). Such an engineered polypeptide can be produced in stages. In the first stage, an intermediate engineered polypeptide containing the modifications of deletion, substitution, insertion, and any combination thereof, can be produced by recombinant techniques as described. Then after an optional purification step as described herein, the intermediate engineered polypeptide is PEGylated (or subjected to other chemical derivatization, e.g., acylation, C-terminal amidation) through chemical modification with an appropriate PEGylating reagent (e.g., from Nektar Transforming Therapeutics, San Carlos, Calif.) to yield the engineered polypeptide derivative. One skilled in the art will appreciate that the procedure described herein can apply to an engineered polypeptide containing a combination of modifications selected from deletion, substitution, insertion, derivation, and other means of modification well known in the art.

C-terminal amidation can be achieved by use of a glycine amino acid-C-terminally extended precursor, synthesized for example in yeast (e.g. Pichia) as alpha-factor fusion protein that will be secreted into culture medium. After purification, the C-terminal glycine of the engineered polypeptide precursor can be converted to amide by enzymatic amidation, e.g. peptidylglycine alpha-amidating monooxygenase (PAM). See e.g., Cooper et al., 1989, Biochem. Biophys. Acta, 1014:247-258. See also U.S. Pat. No. 6,319,685, which teaches methods for enzymatic amidation, including an alpha-amidating enzyme from rat being sufficiently pure in alpha-amidating enzyme to exhibit a specific activity of at least about 25 mU per mg of protein, and being sufficiently free of proteolytic impurities to be suitable for use with substrates purified from natural sources or produced by recombinant DNA techniques.

A variety of peptide purification techniques known in the art may be utilized. Similarly, methods are available for assaying the level of protein expression by a host cell. Numerous methods are also known for isolation of expressed polypeptides from conditioned media. For example, a host cell (e.g., a mammalian host cell) can be engineered to express and secrete a recombinant polypeptide described herein. The recombinant polypeptide can then be purified from the conditioned media using conventional techniques, such as immunoprecipitation, affinity chromatography, polypeptide A-sepharose, gel electrophoresis, and the like.

### Adeno-Associated Viral (AAV) Vectors

In one aspect, the invention provides an AAV vector comprising a polynucleotide of the invention.

In embodiments, the AAV vector is in the form of an AAV vector particle. For example, the AAV vector particle can comprise an AAV2 genome, an AAV8 genome, or an AAV9 genome.

In embodiments, the AAV vector particle comprises AAV9 capsid proteins or AAV8 capsid proteins.

Methods of preparing and modifying viral vectors and viral vector particles, such as those derived from AAV, are well known in the art.

The AAV vector may comprise an AAV genome or a fragment or derivative thereof.

AAV is known to be capable of packaging genomes up to 5.2 kb in size (Dong, J.-Y. et al. (1996) Human Gene Therapy 7:2101-2112).

An AAV genome is a polynucleotide sequence, which may encode functions needed for production of an AAV particle. These functions include those operating in the replication and packaging cycle of AAV in a host cell, including encapsidation of the AAV genome into an AAV particle. Naturally occurring AAVs are replication-deficient and rely on the provision of helper functions in trans for completion of a replication and packaging cycle. Accordingly, the AAV genome of the AAV vector of the invention is typically replication-deficient.

The AAV genome may be in single-stranded form, either positive or negative-sense, or alternatively in double-stranded form. The use of a double-stranded form allows bypass of the DNA replication step in the target cell and so can accelerate transgene expression.

The AAV genome may be from any naturally derived serotype, isolate or clade of AAV. Thus, the AAV genome may be the full genome of a naturally occurring AAV. As is known to the skilled person, AAVs occurring in nature may be classified according to various biological systems.

Commonly, AAVs are referred to in terms of their serotype. A serotype corresponds to a variant subspecies of AAV which, owing to its profile of expression of capsid surface antigens, has a distinctive reactivity which can be used to distinguish it from other variant subspecies. Typically, a virus having a particular AAV serotype does not efficiently crossreact with neutralizing antibodies specific for any other AAV serotype.

AAV serotypes include AAV1, AAV2, AAV3, AAV4, AAV5, AAV6, AAV7, AAV8, AAV9, AAV10 and AAV11, and also recombinant serotypes, such as Rec2 and Rec3, recently identified from primate brain. Any of these AAV serotypes may be used in the invention.

In some embodiments, the AAV vector particle is an AAV1, AAV2, AAV3, AAV4, AAV5, AAV6, AAV7, AAV8, AAV9, AAV10, AAV11, Rec2 or Rec3 AAV vector particle.

In some embodiments, the AAV is an AAV1, AAV2, AAV5, AAV7, AAV8 or AAV9 serotype.

The capsid protein may be a mutant capsid protein such as disclosed in WO 2008/124724, which is herein incorporated by reference.

Reviews of AAV serotypes may be found in Choi et al. (2005) Curr. Gene Ther. 5:299-310 and Wu et al. (2006) Molecular Therapy 14:316-27. The sequences of AAV genomes or of elements of AAV genomes including ITR sequences, rep or cap genes for use in the invention may be derived from the following accession numbers for AAV whole genome sequences: Adeno-associated virus 1 NC_002077, AF063497; Adeno-associated virus 2 NC_001401; Adeno-associated virus 3 NC_001729; Adeno-associated virus 3B NC_001863; Adeno-associated virus 4 NC_001829; Adeno-associated virus 5 Y18065, AF085716; Adeno-associated virus 6 NC_001862; Avian AAV ATCC VR-865 AY186198, AY629583, NC_004828; Avian AAV strain DA-1 NC_006263, AY629583; Bovine AAV NC_005889, AY388617.

AAV may also be referred to in terms of clades or clones. This refers to the phylogenetic relationship of naturally derived AAVs, and typically to a phylogenetic group of AAVs which can be traced back to a common ancestor, and includes all descendants thereof. Additionally, AAVs may be referred to in terms of a specific isolate, i.e. a genetic isolate of a specific AAV found in nature. The term genetic isolate describes a population of AAVs which has undergone limited genetic mixing with other naturally occurring AAVs, thereby defining a recognizably distinct population at a genetic level.

The skilled person can select an appropriate serotype, clade, clone or isolate of AAV for use in the invention on the basis of their common general knowledge. For instance, the AAV5 capsid has been shown to transduce primate cone photoreceptors efficiently as evidenced by the successful correction of an inherited color vision defect (Mancuso et al. (2009) Nature 461:784-7).

The AAV serotype determines the tissue specificity of infection (or tropism) of an AA V. Accordingly, preferred AAV serotypes for use in AA Vs administered to patients in accordance with the invention are those which have natural tropism for or a high efficiency of infection of target cells within the targeted tissue.

Typically, the AAV genome of a naturally derived serotype, isolate or clade of AAV comprises at least one inverted terminal repeat sequence (ITR). An ITR sequence acts in cis to provide a functional origin of replication and allows for integration and excision of the vector from the genome of a cell. In preferred embodiments, one or more ITR sequences flank the nucleotide sequences encoding the protein of the invention. The AAV genome typically also comprises packaging genes, such as rep and/or cap genes which encode packaging functions for an AAV particle. The rep gene encodes one or more of the proteins Rep78, Rep68, Rep52 and Rep40 or variants thereof. The cap gene encodes one or more capsid proteins such as VP1, VP2 and VP3 or variants thereof. These proteins make up the capsid of an AAV particle. Capsid variants are discussed below.

A promoter will be operably linked to each of the packaging genes. Specific examples of such promoters include the p5, p19 and p40 promoters (Laughlin et al. (1979) Proc. Natl. Acad. Sci. USA 76:5567-5571). For example, the p5 and p19 promoters are generally used to express the rep gene, while the p40 promoter is generally used to express the cap gene.

As discussed above, the AAV genome used in the AAV vector of the invention may therefore be the full genome of a naturally occurring AA V. For example, a vector comprising a full AAV genome may be used to prepare an AAV vector or vector particle in vitro. However, while such a vector may in principle be administered to patients, this will rarely be done in practice. Preferably, the AAV genome will be derivatized for the purpose of administration to patients. Such derivatization is standard in the art and the invention encompasses the use of any known derivative of an AAV genome, and derivatives which could be generated by applying techniques known in the art. Derivatization of the AAV genome and of the AAV capsid are reviewed in: Coura and Nardi (2007) Virology Journal 4:99, and in Choi et al. and Wu et al., referenced above.

Derivatives of an AAV genome include any truncated or modified forms of an AAV genome which allow for expression of a transgene from an AAV vector of the invention in vivo. Typically, it is possible to truncate the AAV genome significantly to include minimal viral sequence yet retain the above function. This is preferred for safety reasons to reduce the risk of recombination of the vector with wild-type virus, and also to avoid triggering a cellular immune response by the presence of viral gene proteins in the target cell.

Typically, a derivative will include at least one inverted terminal repeat sequence (ITR), preferably more than one ITR, such as two ITRs or more. One or more of the ITRs may be derived from AAV genomes having different serotypes, or may be a chimeric or mutant ITR. A preferred mutant ITR is one having a deletion of a trs (terminal resolution site). This deletion allows for continued replication of the genome to generate a single stranded genome which contains both coding and complementary sequences, i.e. a self complementary AAV genome. This allows for bypass of DNA replication in the target cell, and so enables accelerated transgene expression.

The one or more ITRs will preferably flank the nucleotide sequence encoding the protein of the invention at either end. The inclusion of one or more ITRs is preferred to aid concatamer formation of the vector of the invention in the nucleus of a host cell, for example following the conversion of single-stranded vector DNA into double-stranded DNA by the action of host cell DNA polymerases. The formation of such episomal concatamers protects the vector construct during the life of the host cell, thereby allowing for prolonged expression of the transgene in vivo.

In embodiments, ITR elements will be the only sequences retained from the native AAV genome in the derivative. Thus, a derivative will preferably not include the rep and/or cap genes of the native genome and any other sequences of the native genome. This is preferred for the reasons described above, and also to reduce the possibility of integration of the vector into the host cell genome. Additionally, reducing the size of the AAV genome allows for increased flexibility in incorporating other sequence elements (such as regulatory elements) within the vector in addition to the transgene.

The following portions could therefore be removed in a derivative of the invention: one inverted terminal repeat (ITR) sequence, the replication (rep) and capsid (cap) genes. However, in some embodiments, derivatives may additionally include one or more rep and/or cap genes or other viral sequences of an AAV genome. Naturally occurring AAV integrates with a high frequency at a specific site on human chromosome 19, and shows a negligible frequency of random integration, such that retention of an integrative capacity in the vector may be tolerated in a therapeutic setting.

Where a derivative comprises capsid proteins i.e. VP1, VP2 and/or VP3, the derivative may be a chimeric, shuffled or capsid-modified derivative of one or more naturally occurring AAVs. In particular, the invention encompasses the provision of capsid protein sequences from different serotypes, clades, clones, or isolates of AAV within the same vector (i.e. a pseudotyped vector).

Chimeric, shuffled or capsid-modified derivatives will be typically selected to provide one or more desired functionalities for the AAV vector. Thus, these derivatives may display increased efficiency of gene delivery, decreased immunogenicity (humeral or cellular), an altered tropism range and/or improved targeting of a particular cell type compared to an AAV vector comprising a naturally occurring AAV genome, such as that of AAV2. Increased efficiency of gene delivery may be effected by improved receptor or co-receptor binding at the cell surface, improved internalization, improved trafficking within the cell and into the nucleus, improved uncoating of the viral particle and improved conversion of a single stranded genome to double-stranded form. Increased efficiency may also relate to an altered tropism range or targeting of a specific cell population, such that the vector dose is not diluted by administration to tissues where it is not needed.

Chimeric capsid proteins include those generated by recombination between two or more capsid coding sequences of naturally occurring AAV serotypes. This may be performed for example by a marker rescue approach in which non-infectious capsid sequences of one serotype are co-transfected with capsid sequences of a different serotype, and directed selection is used to select for capsid sequences having desired properties. The capsid sequences of the different serotypes can be altered by homologous recombination within the cell to produce novel chimeric capsid proteins.

Chimeric capsid proteins also include those generated by engineering of capsid protein sequences to transfer specific capsid protein domains, surface loops or specific amino acid residues between two or more capsid proteins, for example between two or more capsid proteins of different serotypes.

Shuffled or chimeric capsid proteins may also be generated by DNA shuffling or by error-prone PCR. Hybrid AAV capsid genes can be created by randomly fragmenting the sequences of related AAV genes e.g. those encoding capsid proteins of multiple different serotypes and then subsequently reassembling the fragments in a self-priming polymerase reaction, which may also cause crossovers in regions of sequence homology. A library of hybrid AAV genes created in this way by shuffling the capsid genes of several serotypes can be screened to identify viral clones having a desired functionality. Similarly, error prone PCR may be used to randomly mutate AAV capsid genes to create a diverse library of variants which may then be selected for a desired property.

The sequences of the capsid genes may also be genetically modified to introduce specific deletions, substitutions or insertions with respect to the native wild-type sequence. In particular, capsid genes may be modified by the insertion of a sequence of an unrelated protein or peptide within an open reading frame of a capsid coding sequence, or at the N and/or C-terminus of a capsid coding sequence.

The unrelated protein or peptide may advantageously be one which acts as a ligand for a particular cell type, thereby conferring improved binding to a target cell or improving the specificity of targeting of the vector to a particular cell population. The unrelated protein may also be one which assists purification of the viral particle as part of the production process, i.e. an epitope or affinity tag. The site of insertion will typically be selected so as not to interfere with other functions of the viral particle e.g. internalization, trafficking of the viral particle. The skilled person can identify suitable sites for insertion based on their common general knowledge. Particular sites are disclosed in Choi et al., referenced above.

The invention additionally encompasses the provision of sequences of an AAV genome in a different order and configuration to that of a native AAV genome. The invention also encompasses the replacement of one or more AAV sequences or genes with sequences from another virus or with chimeric genes composed of sequences from more than one virus. Such chimeric genes may be composed of sequences from two or more related viral proteins of different viral species.

The AAV vector of the invention may take the form of a nucleotide sequence comprising an AAV genome or derivative thereof and a sequence encoding the protein of the invention.

The AAV particles of the invention include transcapsidated forms wherein an AAV genome or derivative having an ITR of one serotype is packaged in the capsid of a different serotype. The AAV particles of the invention also include mosaic forms wherein a mixture of unmodified capsid proteins from two or more different serotypes makes up the viral capsid. The AAV particle also includes chemically modified forms bearing ligands adsorbed to the capsid surface. For example, such ligands may include antibodies for targeting a particular cell surface receptor.

The AAV vector may comprise multiple copies (e.g., 2, 3 etc.) of the nucleotide sequence referred to herein.

In some embodiments, the polynucleotide further comprises one or more AAV ITRs. In preferred embodiments, the polynucleotide further comprises two AAV ITRs. In some embodiments, the polynucleotide comprises an AAV ITR at its 5′ end and an AA V ITR at its 3′ end. In some embodiments, the AAV ITRs are AAV2, AAV9 or AAV8 ITRs.

### Pharmaceutical Compositions

The recombinant ACE2 polypeptides and fragments thereof, or polynucleotides or vectors as described herein, may be formulated into pharmaceutical compositions. These compositions may comprise, in addition to the medicament, a pharmaceutically acceptable carrier, diluent, excipient, buffer, stabilizer or other materials well known in the art. Such materials should be non-toxic and should not interfere with the efficacy of the active ingredient. The precise nature of the carrier or other material may be determined by the skilled person according to the route of administration.\

According to the administration route chosen, the compositions may be in solid or liquid form, suitable for oral, parenteral, intravenous or intra-arterial administration. The pharmaceutical composition is typically in liquid form. Liquid pharmaceutical compositions generally include a liquid carrier such as water, petroleum, animal or vegetable oils, mineral oil or synthetic oil. Physiological saline solution, magnesium chloride, dextrose or other saccharide solution, or glycols such as ethylene glycol, propylene glycol or polyethylene glycol may be included. In some cases, a surfactant, such as pluronic acid (PF68) 0.001% may be used.

For injection at the site of affliction, the active ingredient may be in the form of an aqueous solution which is pyrogen-free, and has suitable pH, isotonicity and stability. The skilled person is well able to prepare suitable solutions using, for example, isotonic vehicles such as Sodium Chloride Injection, Ringer's Injection or Lactated Ringer's Injection. Preservatives, stabilizers, buffers, antioxidants and/or other additives may be included as required.

For delayed release, the medicament may be included in a pharmaceutical composition which is formulated for slow release, such as in microcapsules formed from biocompatible polymers or in liposomal carrier systems according to methods known in the art.

Such compositions are well-known in the art, see for example Remington's Pharmaceutical Sciences; last edition, Mack Pub.

In embodiments, the pharmaceutical composition can further comprise at least one additional active agent. The phrase “additional active agent” can refer to an agent useful alone, administered simultaneously, administered sequentially, or in combination with one or more additional agents, in the treatment, prophylaxis or palliative care of a subject afflicted with a disease or disorder.

In embodiments, the additional active agent can be employed in the compositions in an amount previously employed alone as a “standard of care”. In embodiments, the additional active agent can be employed in the compositions in less than an amount previously employed alone as a “standard of care”. In embodiments, the additional active agent can be co-administered or employed in a composition of the disclosure in an amount effective to cause measurable reduction of a symptom or sign of a disease, disorder or condition.

### Methods of Treatment

It is to be appreciated that all references herein to treatment include curative, palliative and prophylactic treatment; although in the context of the invention references to preventing are more commonly associated with prophylactic treatment. Treatment may also include arresting progression in the severity of a disease.

The treatment of mammals, particularly humans, is preferred. However, both human and veterinary treatments as described herein are within the scope of the invention.

The administration regime, dosage and posology will be determined by the physician according to his experience, the disease to be treated and the patient's conditions.

The proteins and/or the polynucleotides of the present invention can be administered either singularly or in combination thereof.

The term “combination”, or terms “in combination”, “used in combination with” or “combined preparation” as used herein may refer to the combined administration of two or more agents simultaneously, sequentially or separately.

The term “simultaneous” as used herein means that the agents are administered concurrently, i.e. at the same time.

The term “sequential” as used herein means that the agents are administered one after the other.

The term “separate” as used herein means that the agents are administered independently of each other but within a time interval that allows the agents to show a combined, preferably synergistic, effect. Thus, administration “separately” may permit one agent to be administered, for example, within 1 minute, 5 minutes or 10 minutes after the other.

Embodiments as described herein are drawn towards methods of treating a subject afflicted with a cardiovascular disease or pulmonary disease. In embodiments, the method comprises administering to the subject the recombinant polypeptide described herein, the nucleic acid described herein, the cell described herein, or the pharmaceutical composition described herein.

The term “cardiovascular disease” can refer to a type of disease involving the heart or blood vessels. Cardiovascular diseases include but are not limited to hypertensive heart disease, type 2 diabetes, heart failure, coronary artery disease (CAD), myocardial infarction (commonly known as a heart attack), stroke, rheumatic heart disease, cardiomyopathy, and cardiac arrhythmias (i.e., atrial fibrillation), ventricular tachycardia, cerebrovascular disease, peripheral arterial disease, and arterial thrombosis. In embodiments, the disease or condition comprises a pulmonary disease.

The terms “pulmonary disease” and “respiratory disease” can refer to a type of disease that affects the lungs and other parts of the respiratory system. Pulmonary diseases can be caused by infection, by smoking tobacco, or by breathing in secondhand tobacco smoke, radon, asbestos, or other forms of air pollution. Pulmonary diseases include but are not limited to acute respiratory distress disease (ARDS), COVID-19, asthma, chronic obstructive pulmonary disease (COPD), pulmonary fibrosis, pneumonia, and lung cancer.

The term “treat” or “treatment” can refer to the medical management of a subject with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “prevent,” “preventing,” or “prevention” does not require absolute forestalling of the condition or disease but can also include a reduction in the onset or severity of the disease or condition.

The terms “subject” or “individual” or “animal” or “patient” or “mammal,” can refer to any subject for whom diagnosis, prognosis, or therapy is needed, as described herein. Mammalian subjects can include, but are not limited to, humans, domestic animals, farm animals, zoo animals, sport animals, pet animals such as dogs, cats, guinea pigs, rabbits, rats, mice, horses, cattle, cows; primates such as apes, monkeys, orangutans, and chimpanzees; canids such as dogs and wolves; felids such as cats, lions, and tigers; equids such as horses, donkeys, and zebras; food animals such as cows, pigs, and sheep; ungulates such as deer and giraffes; rodents such as mice, rats, hamsters and guinea pigs; and so on. In certain embodiments, the mammal is a human subject.

In embodiments, the methods comprise administering to the subject a pharmaceutical composition described herein, such as a pharmaceutical composition comprising a recombinant polypeptide or a fragment thereof described herein.

In embodiments, the pharmaceutical composition can be administered in a therapeutically effective amount.” The term “therapeutically effective” can refer to that amount of the composition sufficient to treat a disease and/or ameliorate one or more causes or symptoms of a disease or disorder. Amelioration, for example, only requires a reduction or alteration, not necessarily elimination. As used herein, the terms “therapeutically effective amount” “therapeutic amount” and “pharmaceutically effective amount” are synonymous. One of skill in the art can readily determine the proper therapeutic amount.

The term “administration” or “administering” can refer to the act of physically delivering, e.g., via injection or an oral route, a substance as it exists outside the body into a patient, such as by intracranial, oral, subcutaneous, mucosal, intradermal, intravenous, intramuscular delivery and/or any other method of physical delivery described herein or known in the art. When a disease, disorder or condition, or a symptom thereof, is being treated therapeutically, administration of the substance can occur after the onset of the disease, disorder or condition or symptoms thereof. Prophylactic treatment involves the administration of the substance at a time prior to the onset of the disease, disorder or condition or symptoms thereof.

In embodiments, one or more additional molecules can be administered (i.e., co-administered) to the subject. The phrase “co-administration” can refer to simultaneous and sequential administration of two or more compounds or compositions. An appropriate time course for sequential administration can be chosen by the physician, according to such factors as the nature of a patient's illness, and the patient's condition. Non-limiting examples of additional molecules that can be administered to the subject together with a composition as described herein can comprise conventional therapeutics, such as conventional cardiovascular disease therapeutics or pulmonary disease therapeutics.

Conventional cardiovascular disease therapeutics include, but are not limited to, anticoagulants, antiplatelet agents and dual antiplatelet therapy, ACE inhibitors, angiotensin II receptor blockers, angiotensin receptor-neprilysin inhibitors, beta blockers, calcium channel blockers, cholesterol-lowering medications, digitalis preparations, diuretics, and vasodilators. Conventional pulmonary disease therapeutics include, but are not limited to, oxygen therapy, pulmonary rehabilitation, and thoracentesis.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The invention will be further described in the following examples, which do not limit the scope of the invention described in the claims.

### EXAMPLES

Examples are provided herein to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

ACE2-5R: an ACE2 mutant with enhanced activity

A new human Angiotensin Converting Enzyme type 2 (ACE2) protein (isoform 1, uniprot id: Q9BYF1) was generated by mutation of five lysine residues into arginine residues in the carboxy tail of the protein, specifically 769KKK771, 773K and 787K were substituted to 769RRR771, 773R and 787R. As a result, this mutant protein is resistant to ubiquitination and angiotensin II-induced degradation, displays basal enhanced activity and increased cellular expression levels.

Aspects of the invention can be used for gene therapy to boost ACE2 activity and oppose the development of cardiovascular and pulmonary diseases such as hypertension, type 2 diabetes, heart failure, ARDS and COVID-19.

Embodiments as described herein are resistant to ubiquitination and therefore cannot be degraded via this mechanism, providing the protein with increased half-life and enhanced enzymatic activity toward formation of angiotensin (1-7) which is known to have beneficial effects on cardiovascular and pulmonary function.

We will validate the effects observed in vitro and in vivo, at cellular and animal levels, on humans.

### Example 2

Nedd4-2 Up-Regulation is Associated with ACE2 Ubiquitination in Hypertension

### Abstract

### Background

Angiotensin-converting enzyme 2 (ACE2) is a critical component of the compensatory renin-angiotensin system that is down-regulated during the development of hypertension, possibly via ubiquitination. However, little is known about the mechanisms involved in ACE2 ubiquitination in neurogenic hypertension. This study aimed at identifying ACE2 ubiquitination partners, establishing causal relationships and clinical relevance, and testing a gene therapy strategy to mitigate ACE2 ubiquitination in neurogenic hypertension.

### Methods

Bioinformatics and proteomics were combined to identify E3 ubiquitin ligases associated with ACE2 ubiquitination in chronically hypertensive mice. In vitro gain/loss of function experiments assessed ACE2 expression and activity to validate the interaction between ACE2 and the identified E3 ligase. Mutation experiments were further used to generate a ubiquitination-resistant ACE2 mutant (ACE2-5R). Optogenetics, blood pressure telemetry, pharmacological blockade of GABAA receptors in mice expressing ACE2-5R in the bed nucleus of the stria terminalis (BNST), and capillary western analysis were used to assess the role of ACE2 ubiquitination in neurogenic hypertension. Ubiquitination was first validated as leading to ACE2 down-regulation, and Neural precursor cell-expressed developmentally down-regulated protein 4-2 (Nedd4-2) was identified as a E3 ligase up-regulated in hypertension and promoting ACE2 ubiquitination. Mutation of lysine residues in the C-terminal of ACE2 was associated with increased activity and resistance to angiotensin (Ang)-II-mediated degradation. Mice transfected with ACE2-5R in the BNST exhibited enhanced GABAergic input to the paraventricular nucleus (PVN) and a reduction in hypertension. ACE2-5R expression was associated with reduced Nedd4-2 levels in the BNST.

### Results

Ubiquitination was first validated as leading to ACE2 downregulation and Nedd4-2 identified as a E3 ligase up-regulated in hypertension and promoting ACE2 ubiquitination. Mutation of lysine residues in the C-terminal of ACE2 was associated with increased activity and resistance to Ang-II-mediated degradation. Mice transfected with ACE2-5R in the BNST exhibited enhanced GABAergic input to the paraventricular nucleus (PVN) and reduction of hypertension. ACE2-5R expression was associated with reduced Nedd4-2 levels in the BNST.

### Conclusion

Our data identify Nedd4-2 as the first E3 ubiquitin ligase involved in ACE2 ubiquitination in Ang-II-mediated hypertension. We demonstrate the pivotal role of ACE2 on GABAergic neurons in the maintenance of an inhibitory tone to the PVN and the regulation of pre-sympathetic activity. These findings provide a new working model where Nedd4-2 could contribute to ACE2 ubiquitination, leading to the development of neurogenic hypertension and highlighting potential novel therapeutic strategies.

### Introduction

Angiotensin-converting enzyme 2 (ACE2) is a member of the renin-angiotensin system (RAS), primarily involved in the conversion of the vasoconstrictor octapeptide angiotensin (Ang)-II to the vasodilatory heptapeptide Ang-(1-7). Decreases in cellular ACE2 levels have been associated with cardiovascular diseases like hypertension and diabetes, while overexpression of ACE2 has been shown to have beneficial effects in preventing these diseases.^{1,2} Our group previously identified several post-translational mechanisms mediated by the Ang-II type 1 receptor (AT1R), involved in ACE2 down-regulation and their contribution to the development of neurogenic hypertension, including ACE2 shedding³ and internalization followed by degradation in lysosomes.^{4,5} Importantly, we observed that this latter mechanism might be associated with ubiquitination induced by elevated Ang-II levels.⁴ Despite recent reports identifying murine double minute 2 (MDM2) and S-phase kinase-associated protein 2 (Skp2) as important E3 ligases for ACE2 ubiquitination in the lung,^{6,7} there is a general gap in knowledge regarding ACE2 ubiquitination and its implications, notably for cardiovascular diseases such as hypertension.

Ubiquitination is a general post-translational modification of proteins consisting of the addition of ubiquitin, a 76-amino-acid polypeptide, to substrate proteins, leading to either degradation in proteasomes or digestion in lysosomes. The latter is typical for plasma membrane proteins and is of utmost importance in the regulation of cellular signaling. Ubiquitin is covalently attached by its C-terminus to specific lysine residues on the target protein,^{8,9} a process mediated by the sequential action of three types of enzymes: E1 ubiquitin-activating enzymes, E2 ubiquitin-conjugating enzymes, and E3 ubiquitin ligases. 10Ubiquitin is first activated by E1 in an adenosine triphosphate (ATP)-dependent manner and then transferred to a cysteine residue at the active site of E2. Finally, the E3 ligase directly or indirectly catalyzes the covalent attachment of ubiquitin to the target protein.¹⁰ The human genome encodes two E1 enzymes, about 60 different E2 enzymes, and more than 600 E3 ubiquitin ligases. Ubiquitin can be attached as single or multiple residues to the substrate protein, resulting in mono- or poly-ubiquitination, respectively, and is essential for controlling the expression level of substrate proteins.

The brain RAS contributes to the regulation of cardiovascular function, and overactivation of this system, notably in hypertension, is well known to lead to enhanced pre-sympathetic activity of glutamatergic neurons in forebrain nuclei, such as the paraventricular nucleus (PVN).11 We previously observed that ACE2 is expressed on GABAergic neurons and a lack of ACE2 leads to impaired inhibitory input to the PVN, leading to enhanced sympathetic activity to peripheral end organs. 12 The activity of pre-sympathetic PVN neurons is under the influence of GABAergic input originating from outside the PVN. The bed nucleus of the stria terminalis (BNST) is a region rich in GABAergic neurons known to regulate cardiovascular parameters, notably during stress.¹³ Our group previously reported that ACE2 is expressed in the BNST¹⁴ and more recently suggested that it contributes to the inhibitory tone to the PVN.¹² However, in addition to the lack of knowledge with regard to ACE2 ubiquitination partners, there is also limited information on the impact of ubiquitination, notably within the compensatory RAS, on the regulation of sympathetic activity and the development of neurogenic hypertension. To address these gaps in knowledge, we aimed to identify E3 ligases regulating ACE2 ubiquitination and investigated this mechanism in the context of neurogenic hypertension.

In the present study, we identified Neural precursor cell-expressed developmentally down-regulated protein 4-2 (Nedd4-2) as an E3 ubiquitin ligase targeting lysine residues in the carboxy tail of ACE2. Following in vitro and in vivo validation of Nedd4-2 interaction with ACE2, we used a ubiquitination-resistant ACE2 mutant for site-specific gene therapy to the brain to highlight the detrimental role of constitutive ACE2 ubiquitination in the activity of inhibitory GABAergic neurons and demonstrated that this mechanism contributes to the development of neurogenic hypertension.

### Results

### Ubiquitination Reduces ACE2 Expression and Activity

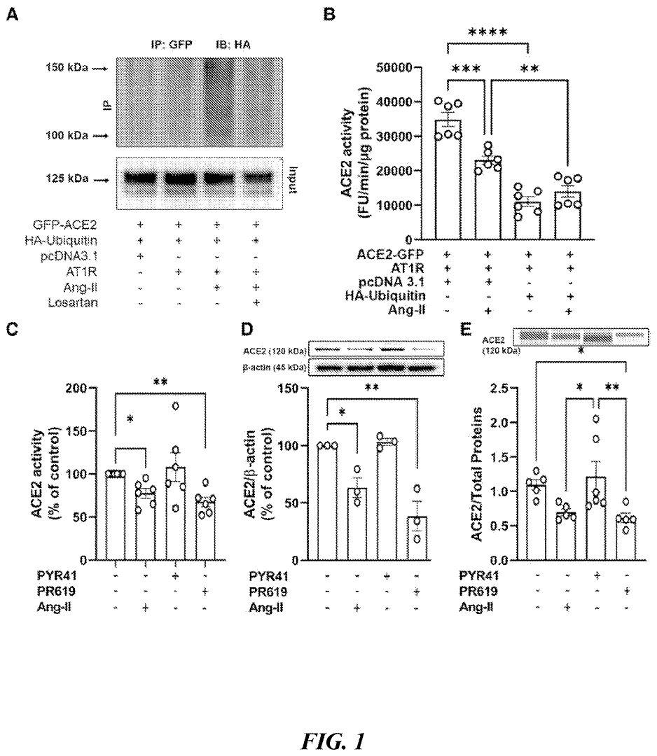

We previously reported that Ang-II activation of AT1R leads to a reduction in ACE2 plasma membrane localization and total cellular expression levels, possibly through ubiquitination.⁴ To confirm that this mechanism is indeed critical for the regulation of ACE2 expression and activity, we first assessed ACE2 ubiquitination levels by co-immunoprecipitation in HEK293T cells transfected with ACE2, AT1R, and ubiquitin (FIG. 1, panel A). As expected, Ang-II treatment (100 nM, 4 h) involves AT1R-dependent ACE2 ubiquitination, as this effect was blocked by the AT1R antagonist losartan and was not observed in cells not transfected with AT1R (FIG. 1, panel A, right). This suggests that AT1R activation is required for Ang-II-mediated ubiquitination of ACE2, extending our previous observations that ACE2 is not internalized by Ang-II in cells not expressing AT1R.^{4,5} In addition, overexpression of ubiquitin resulted in reduced ACE2 activity (FIG. 1, panel B). The seemingly contrasting observations that in cells transfected with ubiquitin, treatment with Ang-II did not further decrease ACE2 activity (FIG. 1, panel B) might suggest that ACE2 ubiquitination can occur both constitutively and in the presence of Ang-II.

The importance of ubiquitination in regulating ACE2 expression and activity is further supported by specific inhibition of ubiquitination and deubiquitination. PYR-41, a cell-permeable inhibitor of E1 ubiquitin-activating enzymes (50 μM for 2 h), did not affect basal ACE2 activity or expression (FIG. 1, panels C and D). In contrast, pre-treatment with PR-619, a broad-spectrum reversible inhibitor of cysteine-reactive deubiquitinating enzymes (20 μM, 6 h), reduced both ACE2 activity (FIG. 1, panel C) and protein expression (FIG. 1, panel D) to levels similar to those achieved by Ang-II treatment, suggesting that deubiquitination is critical to maintaining ACE2 expression and activity. To ensure that these results are not dependent on the amount of plasmids transfected, we repeated this experiment in Neuro2A cells (FIG. 1, panel E) and confirmed that ACE2 deubiquitination is a constitutive process that maintains ACE2 expression. Together, these data suggest that ubiquitination and deubiquitination are critical mechanisms for the regulation of ACE2 expression.

### Nedd4-2 is Up-Regulated in Hypertension

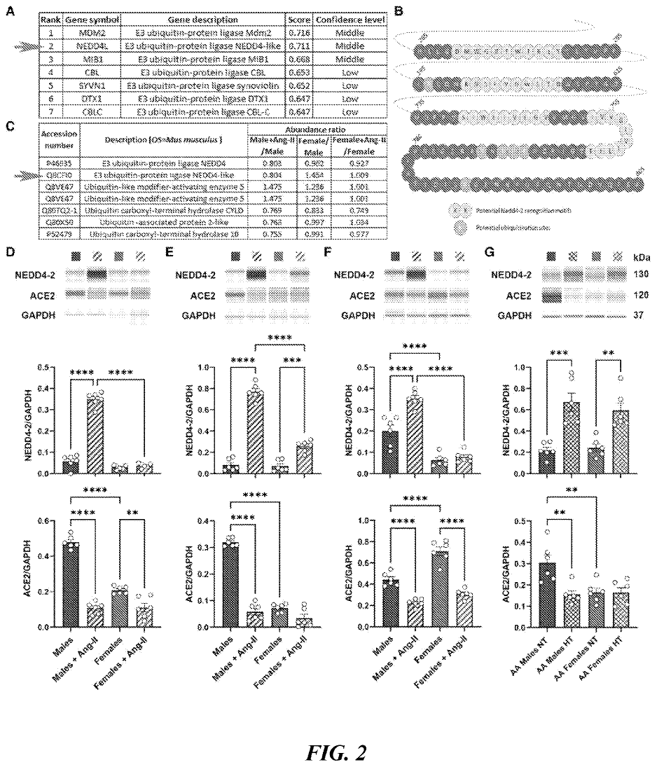

A multipronged discovery strategy was then implemented to identify E3 ubiquitin ligases interacting with ACE2 in neurogenic hypertension. A predictive bioinformatic analysis, to identify key E3 ubiquitin ligases involved in ACE2 ubiquitination, revealed that Nedd4-2 (also known as NEDD4L) has the highest confidence level for interaction with ACE2 (FIG. 2, panel A) after MDM2, another E3 ligase previously reported to ubiquitinate ACE2.6 Four potential Nedd4-2 recognition motifs were identified within the ACE2 amino acid sequence, including two in the C-terminal, next to multiple lysine residues (FIG. 2, panel B). To identify changes in E3 ligase expression in neurogenic hypertension, a parallel proteomic analysis was performed using hypothalamic samples isolated from mice infused with Ang-II. Again, Nedd4-2 was highlighted as a positive hit with potential changes in expression affected by both sex and hypertension (FIG. 2, panel C). To validate this finding and determine tissue specificity, Nedd4-2 and ACE2 protein expression levels were assessed in various organs affected by neurogenic hypertension. Basal ACE2 expression was higher in the brain (FIG. 2, panel D) and heart (FIG. 2, panel E) of males, but Nedd4-2 was not different between sexes in these tissues. In the kidney, however, lower Nedd4-2 levels in females were associated with higher ACE2 expression (FIG. 2, panel F). Ang-II-mediated neurogenic hypertension, which was more pronounced in males than in females (Table 1), was associated with a strong Nedd4-2 up-regulation, mostly in males, and a parallel reduction in ACE2 levels in all tissues. However, while ACE2 was reduced in Ang-II-infused females, this was only associated with an up-regulation of Nedd4-2 in the heart, suggesting that ACE2 down-regulation in females might be independent of Nedd4-2.

TABLE 1
Hemodynamic parameters in C57BL6/J mice (n = 9-11) before
and after 4 weeks of Ang-II infusion during their active (9 AM-5 PM)
and resting (9 PM-5 AM) phases.

| | | | Systolic BP | Diastolic BP | Heart Rate |
|---|---|---|---|---|---|
| | | | (mmHg) | (mmHg) | (bpm) |
| Baseline | Active | Males | 125.3 ± 0.6 | 100.9 ± 0.6 | 559.9 ± 2.2 |
| | | Females | 120.5 ± 0.4* | 91.0 ± 0.4* | 579.9 ± 1.8* |
| | Resting | Males | 117.6 ± 0.6 | 98.2 ± 0.7 | 538.7 ± 3.4 |
| | | Females | 108.4 ± 0.4* | 92.0 ± 0.5* | 604.2 ± 2.4* |
| Ang-II infusion | Active | Males | 153.8 ± 1.2^{#} | 125.4 ± 1.0^{#} | 507.2 ± 2.7^{#} |
| | | Females | 143.7 ± 0.9^{#, &} | 113.5 ± 0.9^{#, &} | 551.0 ± 2.7^{#, &} |
| | Resting | Males | 147.0 ± 1.2^{#} | 121.2 ± 1.0^{#} | 485.1 ± 4.5^{#} |
| | | Females | 140.9 ± 0.9^{#, &} | 112.1 ± 0.7^{#, &} | 550.9 ± 4.7^{#, &} |

Two-way analysis of variance followed by Tukey's test for multiple comparisons. Statistical significance:
*P < 0.05 vs. males,
^{#}P < 0.05 vs. baseline and
^{&}P < 0.05 vs. hypertensive males.

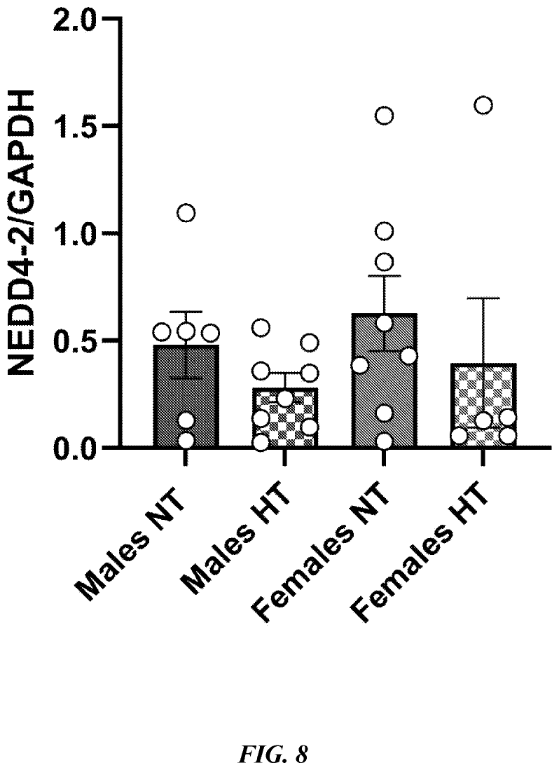

Based on the above lack of tissue specificity and similarities between ACE2 and Nedd4-2 expression in both the brain and the heart, we further examined the clinical relevance of the Nedd4-2/ACE2 relationship in cardiac samples (FIG. 2, panel G) from normotensive and hypertensive donors (Table 2). Surprisingly, Nedd4-2 up-regulation in hypertensive patients and the parallel reduction in ACE2 expression were only observed in the left ventricle of African American males, with no difference in Caucasian donors (FIG. 8). While Nedd4-2 up-regulation was also observed in hypertensive African American females, it was not associated with a reduction in ACE2 levels (FIG. 2, panel G), pointing again to the lack of involvement of Nedd4-2 in ACE2 ubiquitination in females.

TABLE 2
List of donated human cardiac samples.

| Patient | | | | | |
|---|---|---|---|---|---|
| ID | Source | Race | Sex | Age | Comorbidities |

Normotensive patients

| | | | | | |
|---|---|---|---|---|---|
| 13349 | MCW | African | F | 56 | CAD, Tobacco, AFib |
| | | American | | | |
| 11049 | MCW | African | F | 56 | CAD |
| | | American | | | |
| 11546 | MCW | African | F | 53 | CAD, T2DM, Neuropathy, Tobacco |
| | | American | | | |
| 94 | DU | African | F | 37 | Obesity |
| | | American | | | |
| 168 | DU | African | F | 28 | Obesity |
| | | American | | | |
| 307 | DU | African | F | 30 | Tobacco |
| | | American | | | |
| 14131 | MCW | African | M | 58 | Valve disease, Tobacco |
| | | American | | | |
| 14333 | MCW | African | M | 54 | CAD |
| | | American | | | |
| 14554 | MCW | African | M | 43 | Tobacco |
| | | American | | | |
| 84 | DU | African | M | 54 | Tobacco |
| | | American | | | |
| 206 | DU | African | M | 29 | Tobacco |
| | | American | | | |
| 113 | DU | African | M | 22 | None |
| | | American | | | |
| 11048 | MCW | Caucasian | F | 51 | CAD |
| 11070 | MCW | Caucasian | F | 53 | Tobacco, Arthritis, Hypothyroidy |
| 12681 | MCW | Caucasian | F | 51 | CAD, T1DM |
| 9 | DU | Caucasian | F | 66 | None |
| 78 | DU | Caucasian | F | 64 | Tobacco, Obesity |
| 91 | DU | Caucasian | F | 42 | Tobacco |
| 77 | DU | Caucasian | F | 44 | Tobacco, Obesity |
| 85 | DU | Caucasian | F | 49 | Obesity |
| 12688 | MCW | Caucasian | M | 49 | HL, Tobacco, SA, Bipolar |
| 13209 | MCW | Caucasian | M | 45 | HL, Tobacco, Pancreatitis, Alcohol |
| 59 | DU | Caucasian | M | 51 | Tobacco, Bipolar, Obesity |
| 79 | DU | Caucasian | M | 41 | Tobacco, CAD, Obesity |
| 120 | DU | Caucasian | M | 59 | None |
| 150 | DU | Caucasian | M | 52 | Tobacco, Alcohol, GERD, Marijuana |

Hypertensive patients

| | | | | | |
|---|---|---|---|---|---|
| 12266 | MCW | African | F | 50 | CAD, DM, Renal disease |
| | | American | | | |
| 13222 | MCW | African | F | 51 | CAD, HL, Tobacco, AFib, Renal |
| | | American | | | disease, SA, Stroke, Cardiomegaly |
| 14246 | MCW | African | F | 58 | CAD, HL, Tobacco, Epilepsy, SA |
| | | American | | | |
| 9760 | MCW | African | F | 58 | CAD, T2DM, Tobacco |
| | | American | | | |
| 140 | DU | African | F | 61 | Tobacco, Alcohol |
| | | American | | | |
| 34 | DU | African | F | 50 | T2DM, Obesity |
| | | American | | | |
| 12295 | MCW | African | M | 60 | Tobacco |
| | | American | | | |
| 12531 | MCW | African | M | 61 | CAD, T2DM, Tobacco, GERD |
| | | American | | | |
| 13082 | MCW | African | M | 51 | CAD, MI, T2DM, Stroke, SA |
| | | American | | | |
| 90 | DU | African | M | 67 | Tobacco |
| | | American | | | |
| 158 | DU | African | M | 62 | Glaucoma, PTSD |
| | | American | | | |
| 272 | DU | African | M | 55 | None |
| | | American | | | |
| 12630 | MCW | Caucasian | F | 53 | CAD, T2DM, Tobacco, Obesity |
| 13094 | MCW | Caucasian | F | 67 | CAD, HL, Cerebral hemorrhage |
| 65 | DU | Caucasian | F | 51 | None |
| 173 | DU | Caucasian | F | 51 | T2DM |
| 110 | DU | Caucasian | F | 59 | CAD, Tobacco, Obesity |
| 12500 | MCW | Caucasian | M | 65 | CAD |
| 12804 | MCW | Caucasian | M | 53 | CAD, T2DM, Tobacco, Prostate cancer |
| 12917 | MCW | Caucasian | M | 54 | CAD, HL, Epilepsy, Arterial aneurysm |
| 13066 | MCW | Caucasian | M | 63 | CAD, CHF, T2DM, HL |
| 14682 | MCW | Caucasian | M | 68 | AFib, Stroke, Skin cancer, COVID-19 |
| 118 | DU | Caucasian | M | 57 | Gout, HL |
| 123 | DU | Caucasian | M | 59 | HL, Obesity, SA |
| 127 | DU | Caucasian | M | 51 | Obesity |

Left ventricle samples were obtained from the Wisconsin Donor Network and Versiti Blood Center of Wisconsin organ procurement program (MCW) and Duke University Department of Surgery (DU).
Abbreviations:
AFib, atrial fibrillation;
CAD, coronary artery disease;
CHF, chronic heart failure;
GERD, gastroesophageal reflux disease;
HL, hyperlipidemia;
DM, diabetes mellitus;
MI, myocardial infarction;
PTSD, post-traumatic stress disorder;
T1DM, type 1 diabetes mellitus;
T2DM, type 2 diabetes mellitus;
SA, substance abuse.

Together, these data suggest that hypertension is associated with an up-regulation of Nedd4-2 and a parallel decrease in ACE2 expression, predominantly in males. Based on the lack of correlation between ACE2 and Nedd4-2 expression in female mice and patients, ensuing in vivo experiments, targeting Nedd4-2-mediated ubiquitination of ACE2, were conducted only in males.

### ACE2 is Ubiquitinated by Nedd4-2

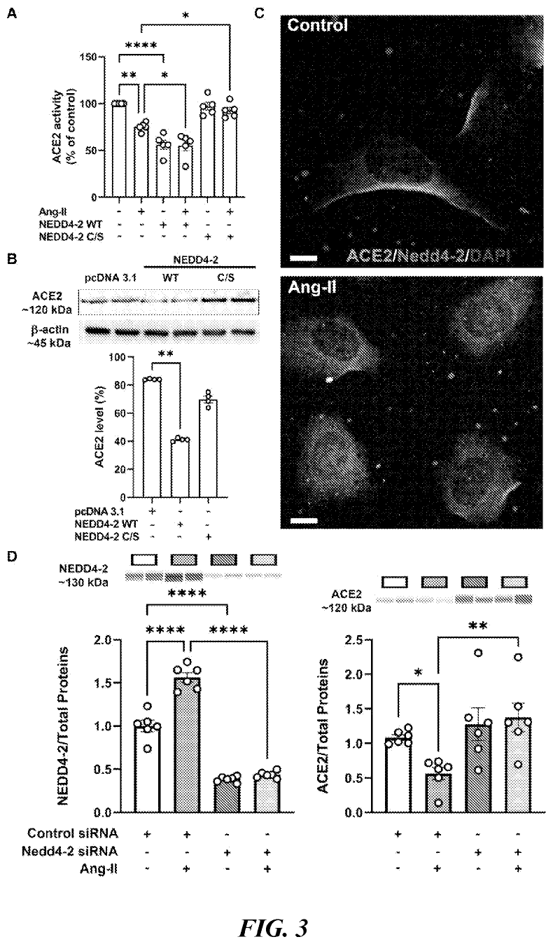

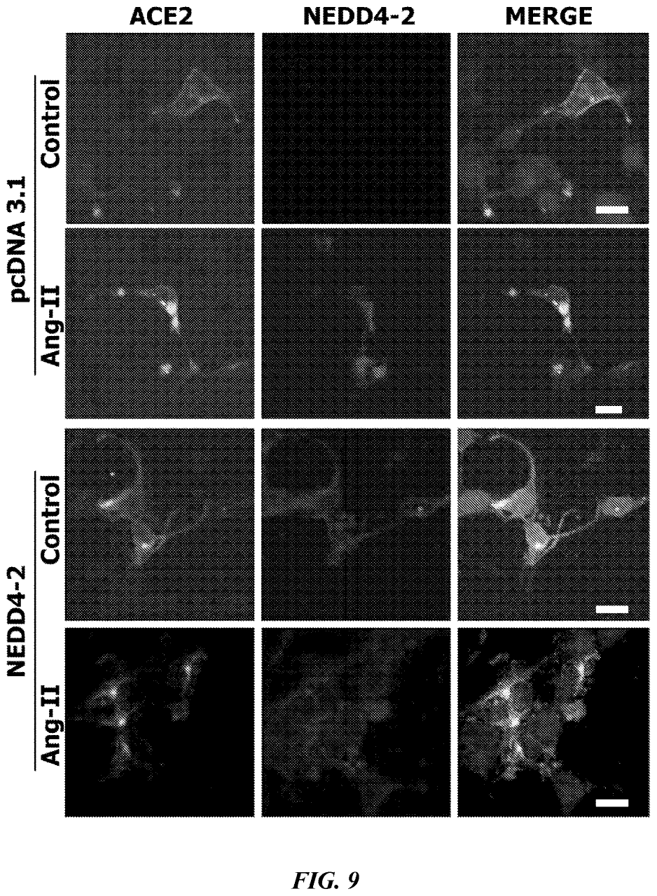

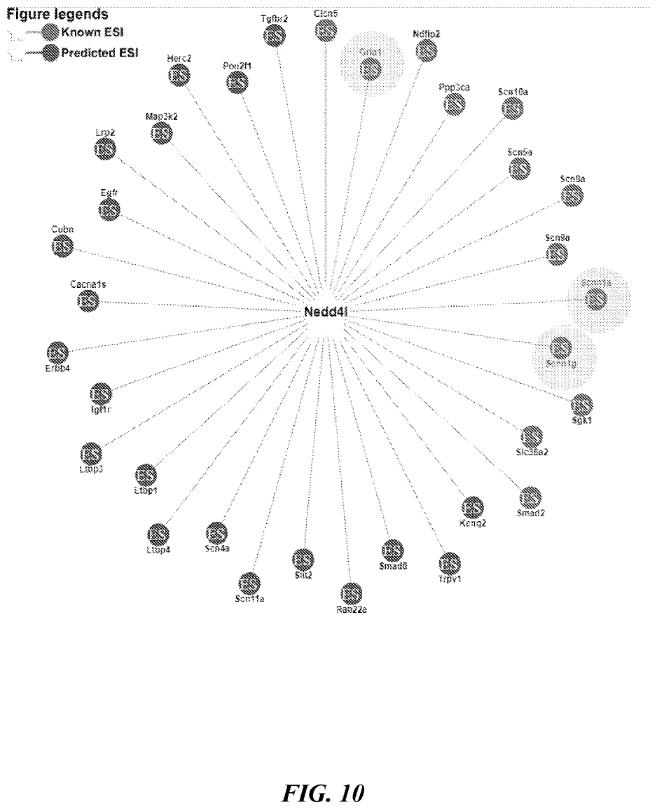

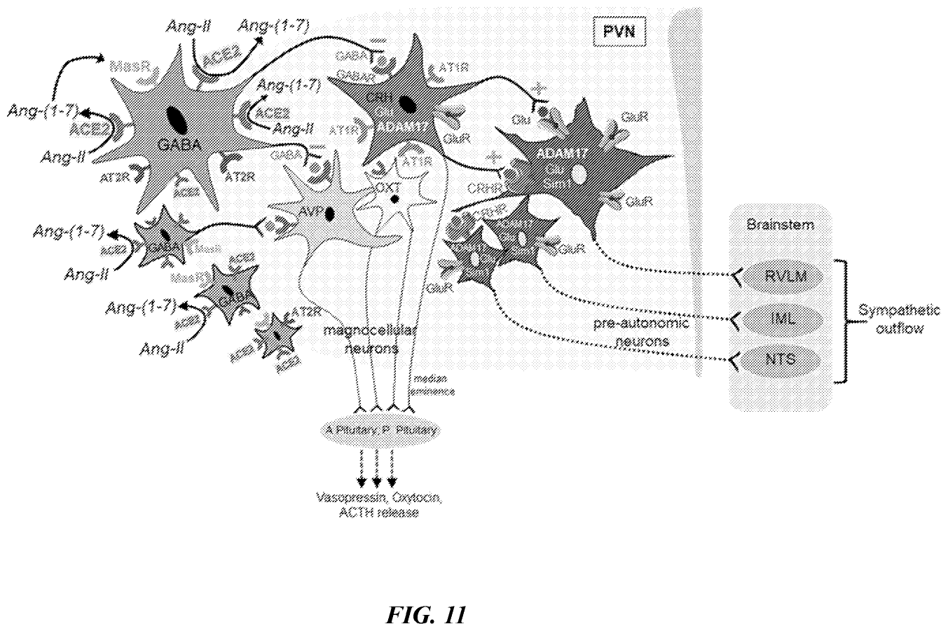

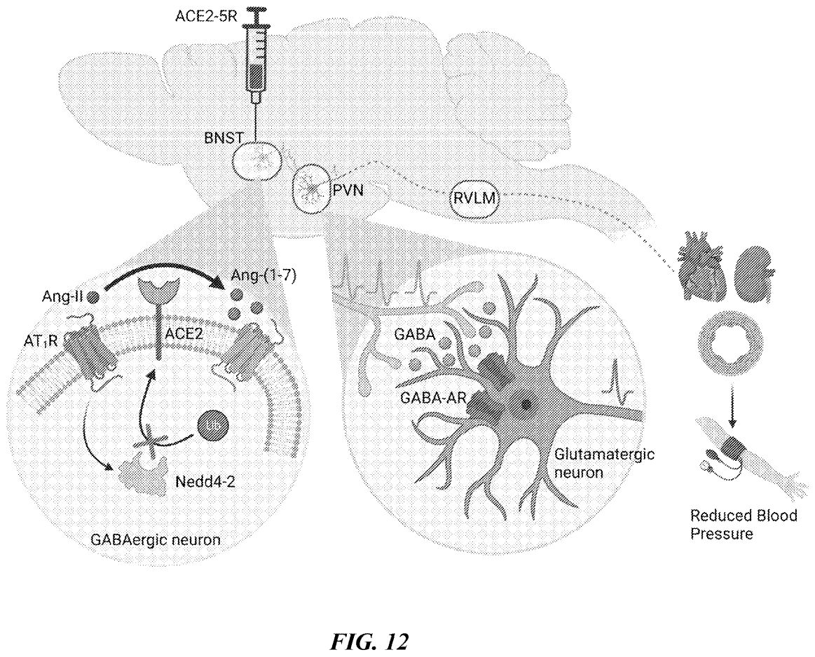

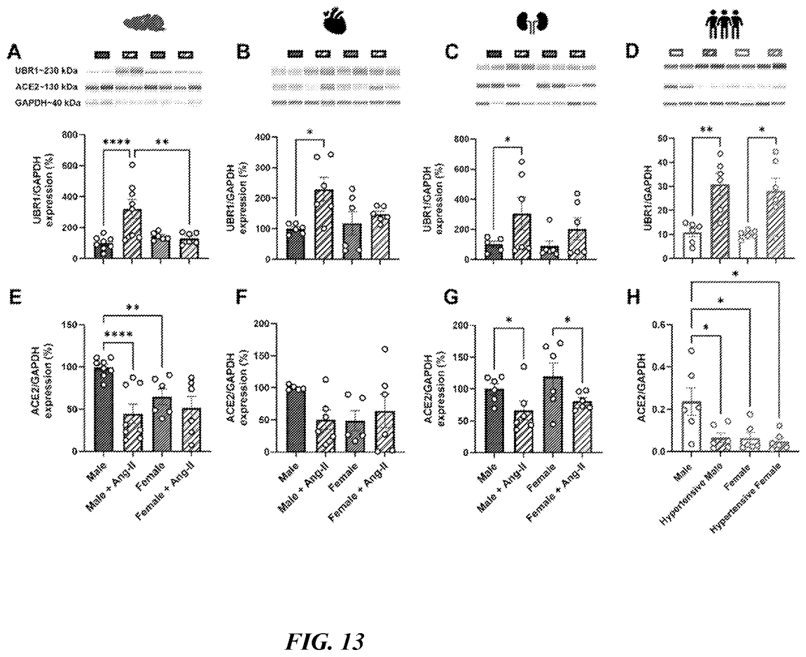

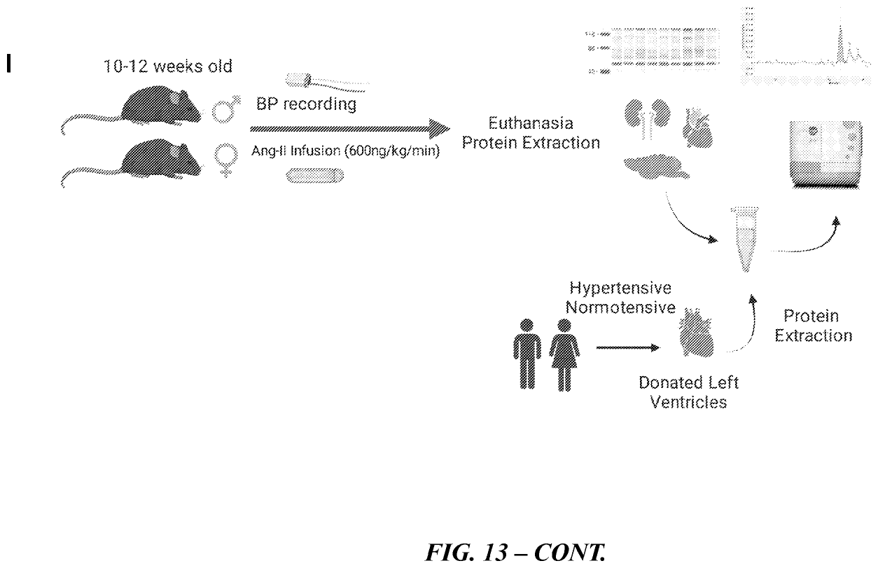

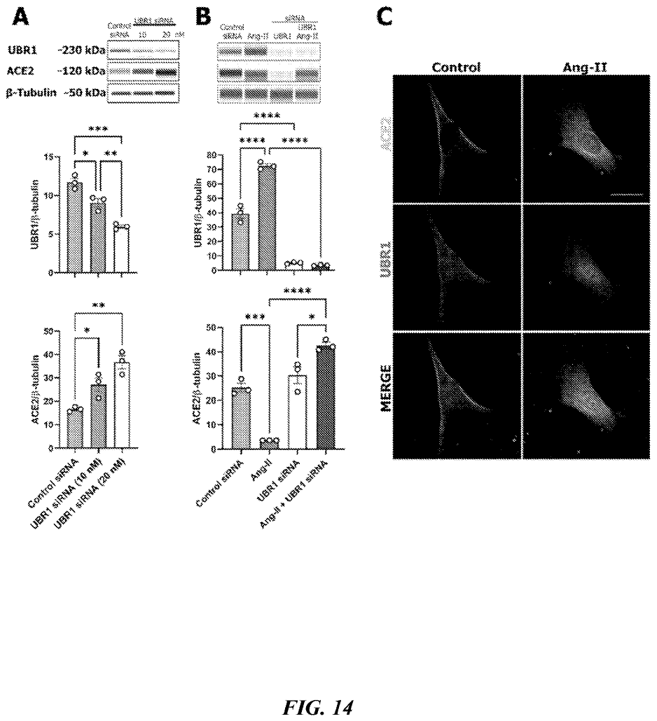

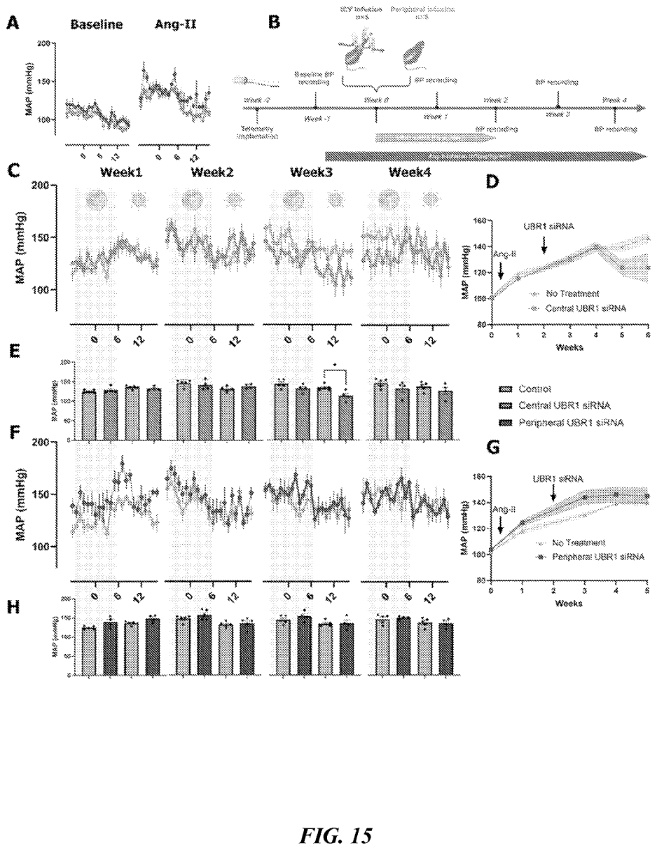

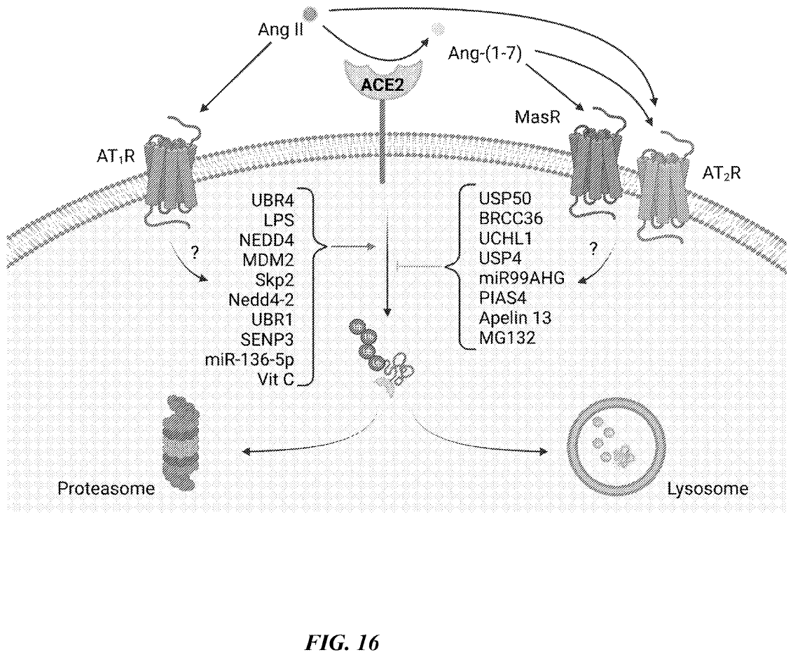

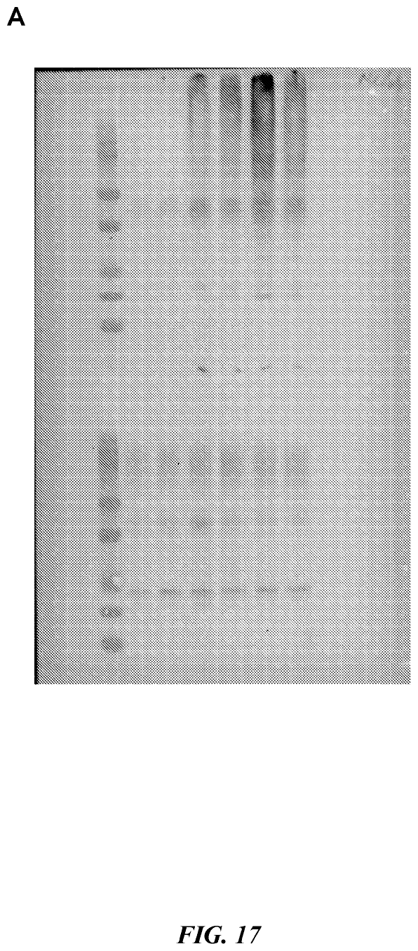

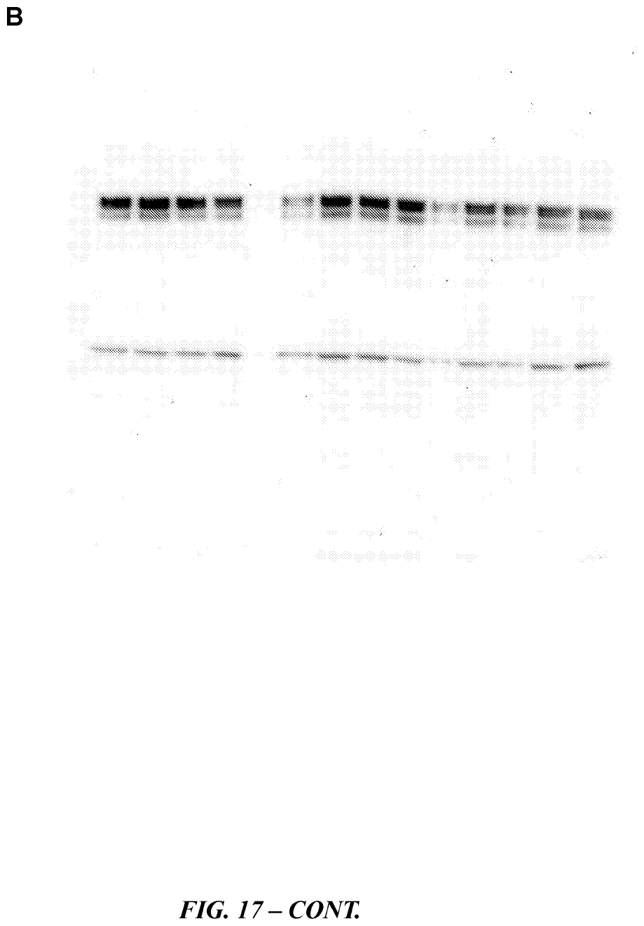

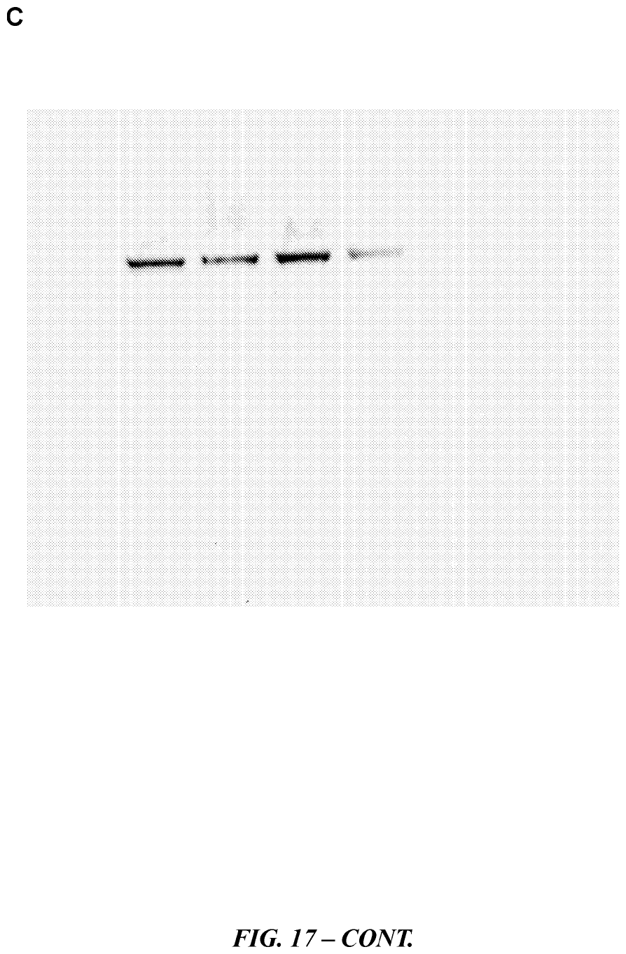

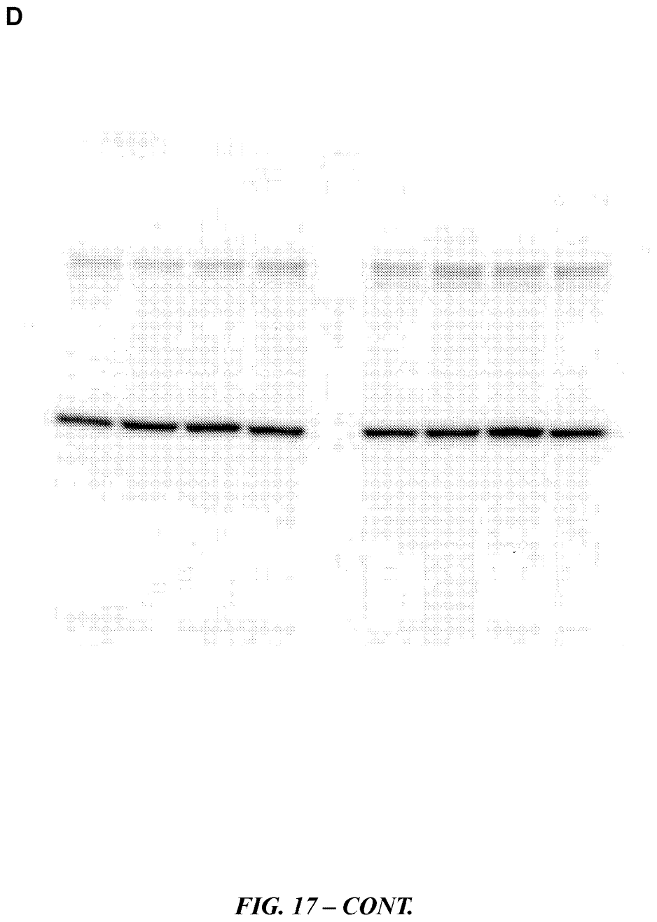

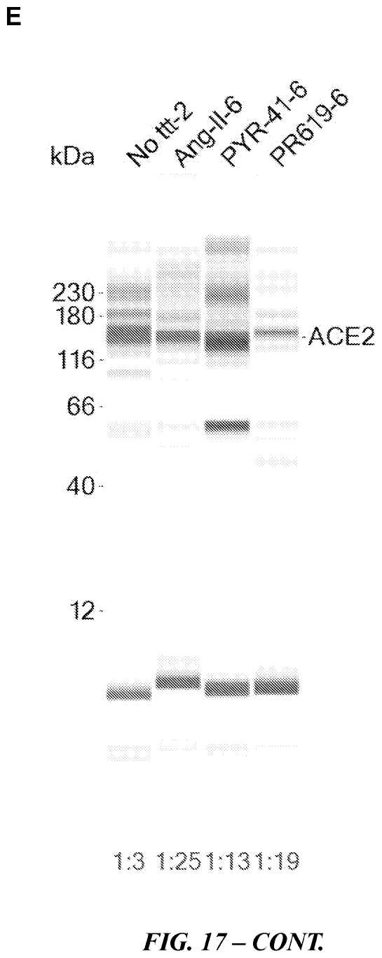

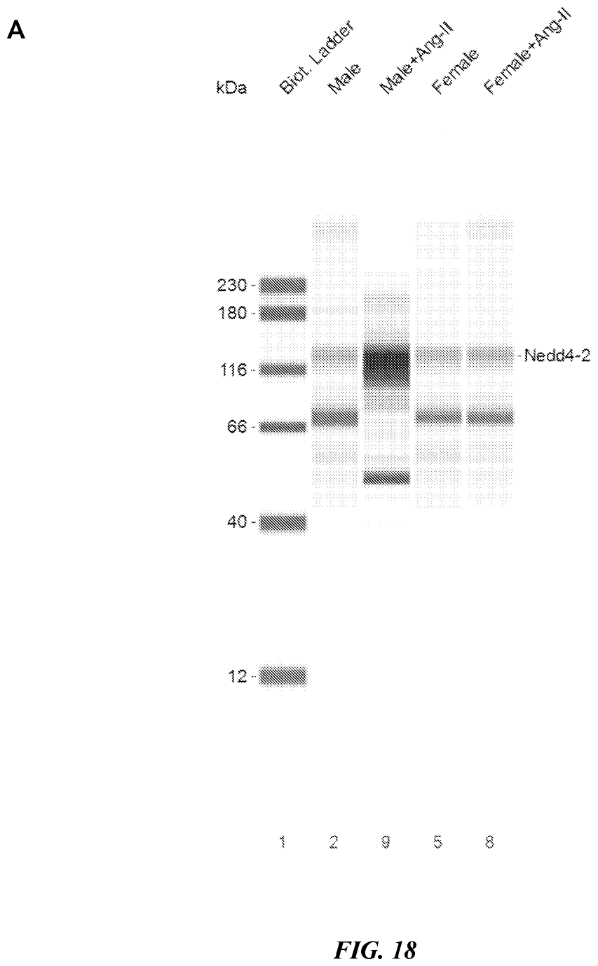

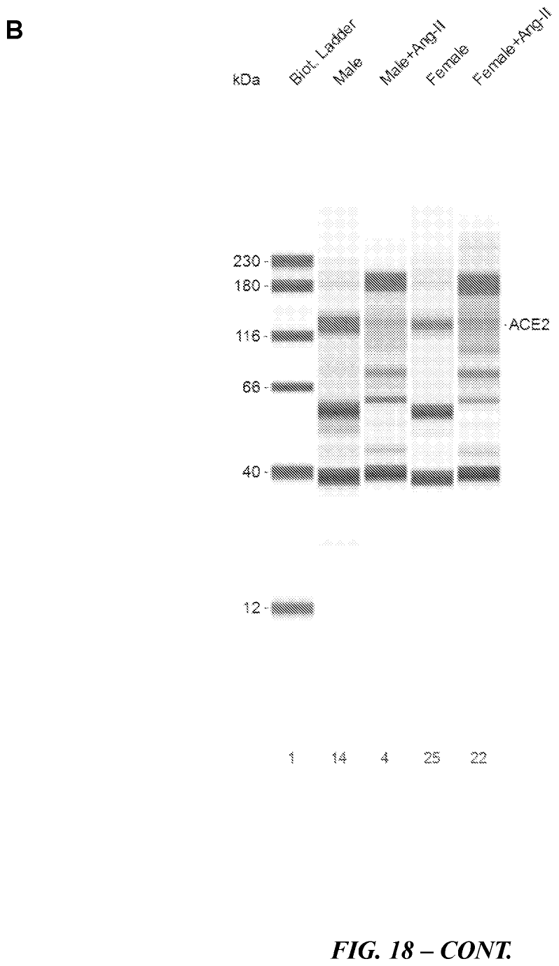

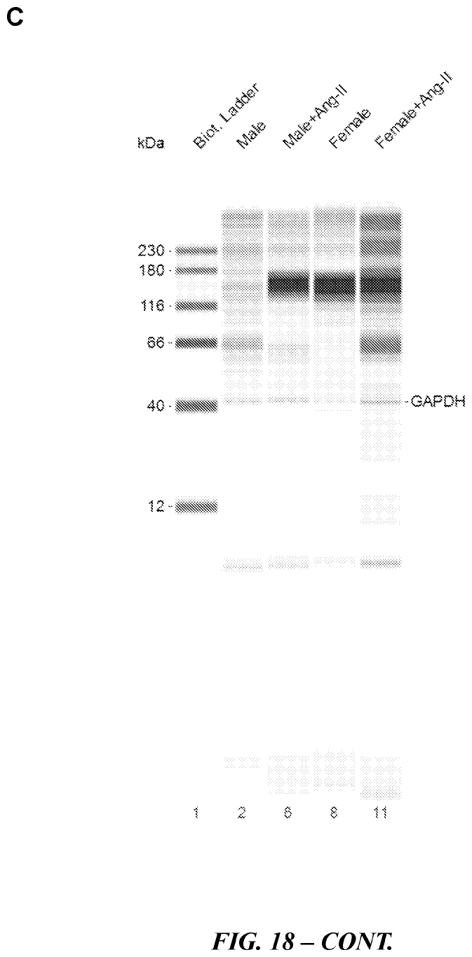

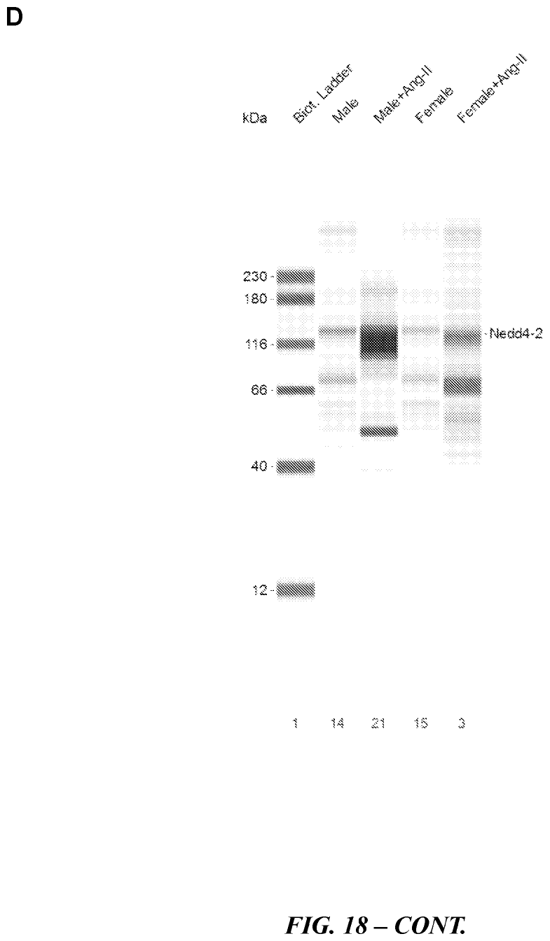

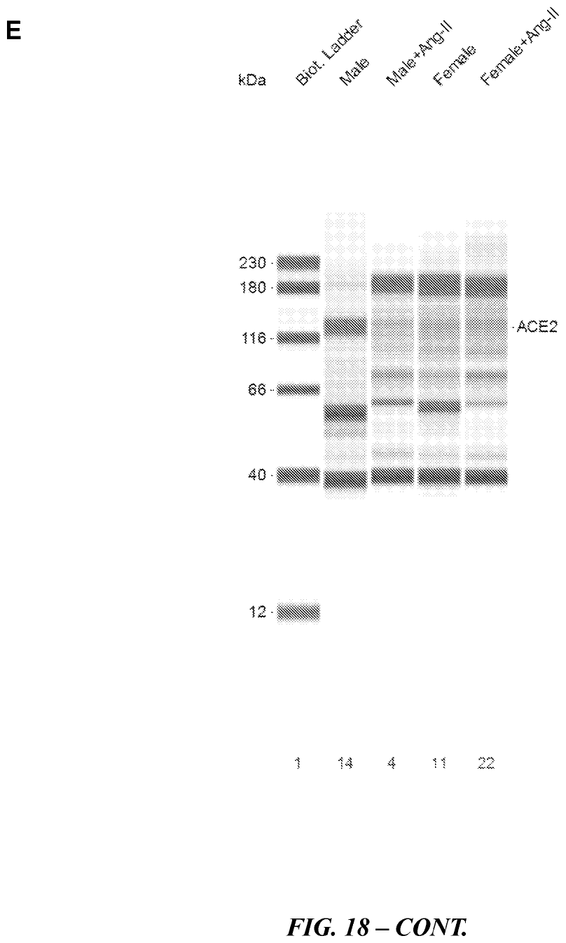

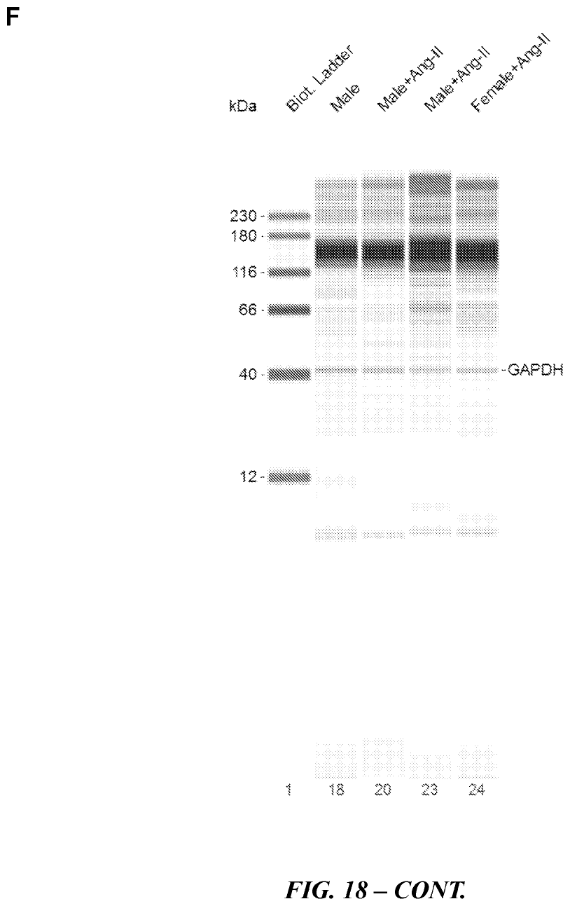

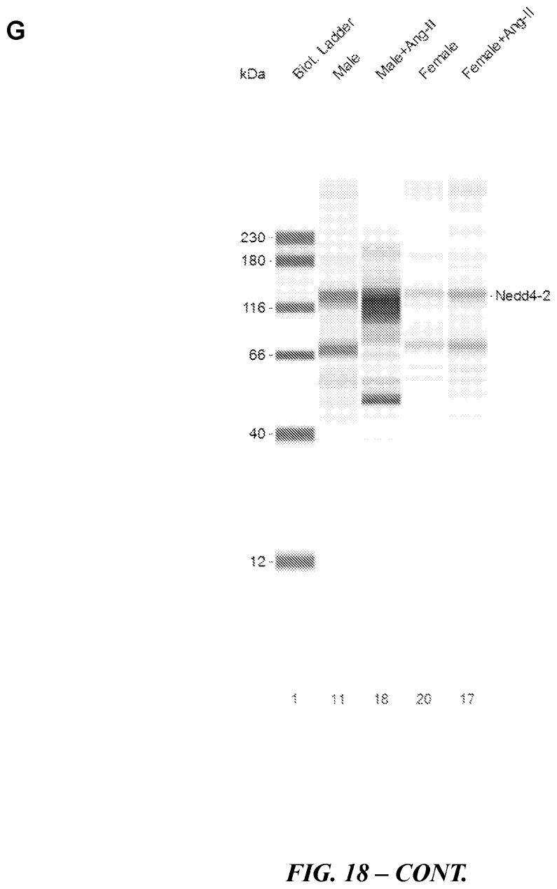

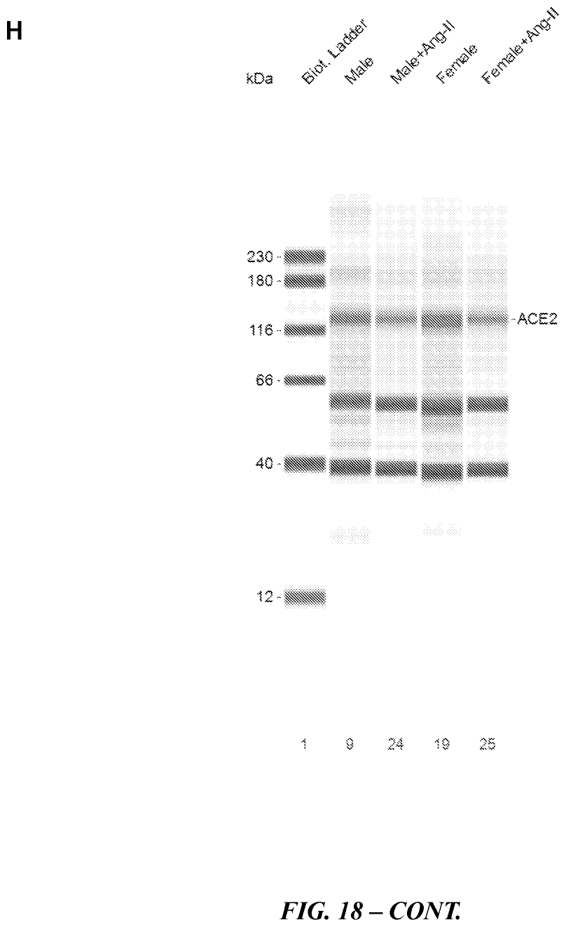

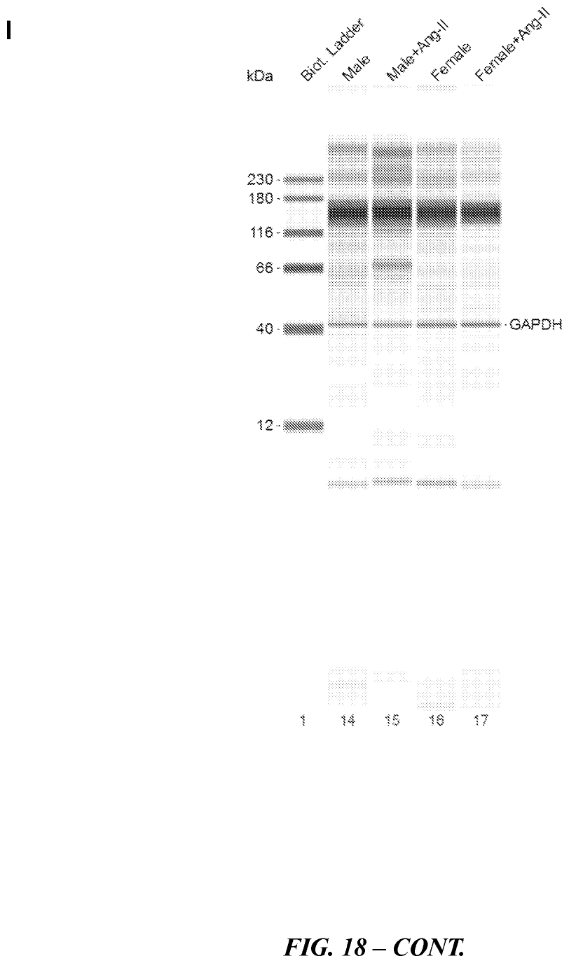

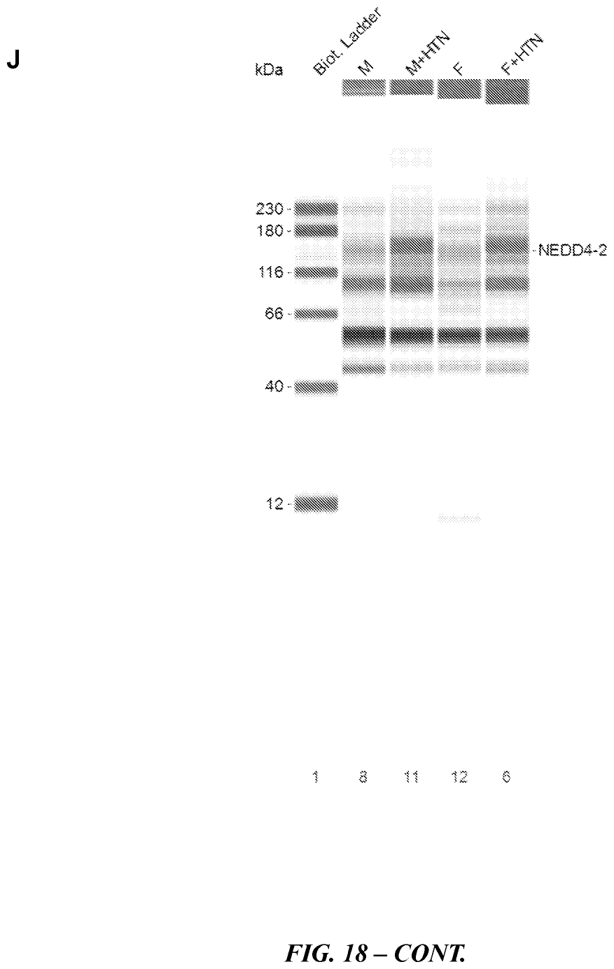

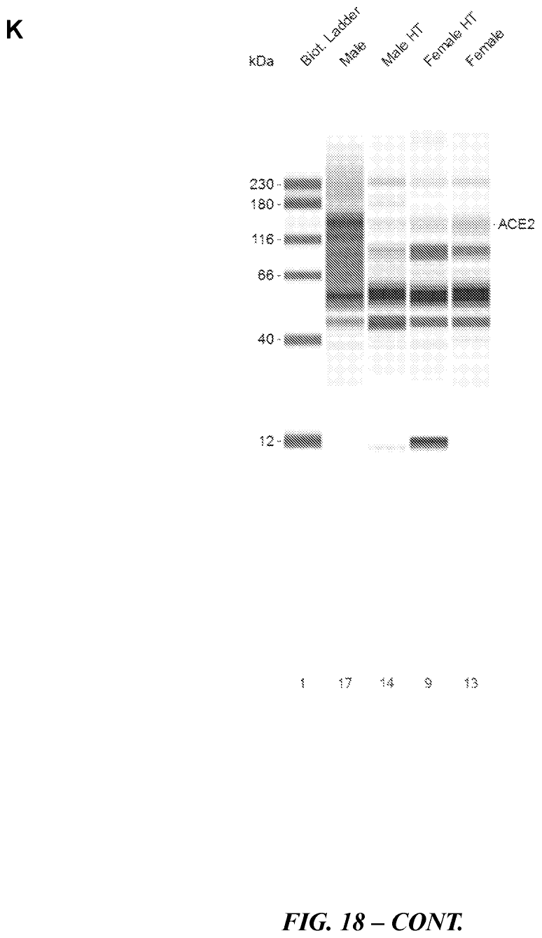

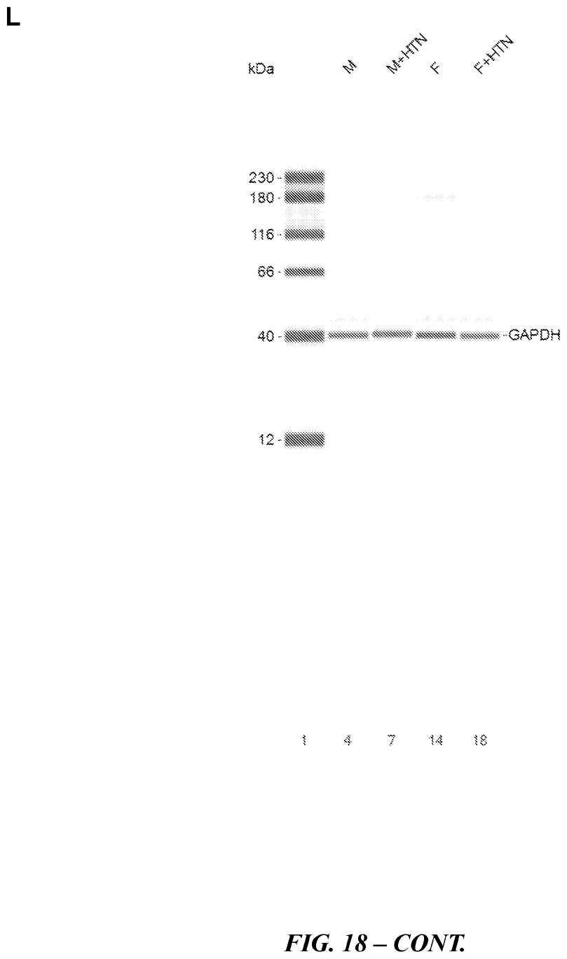

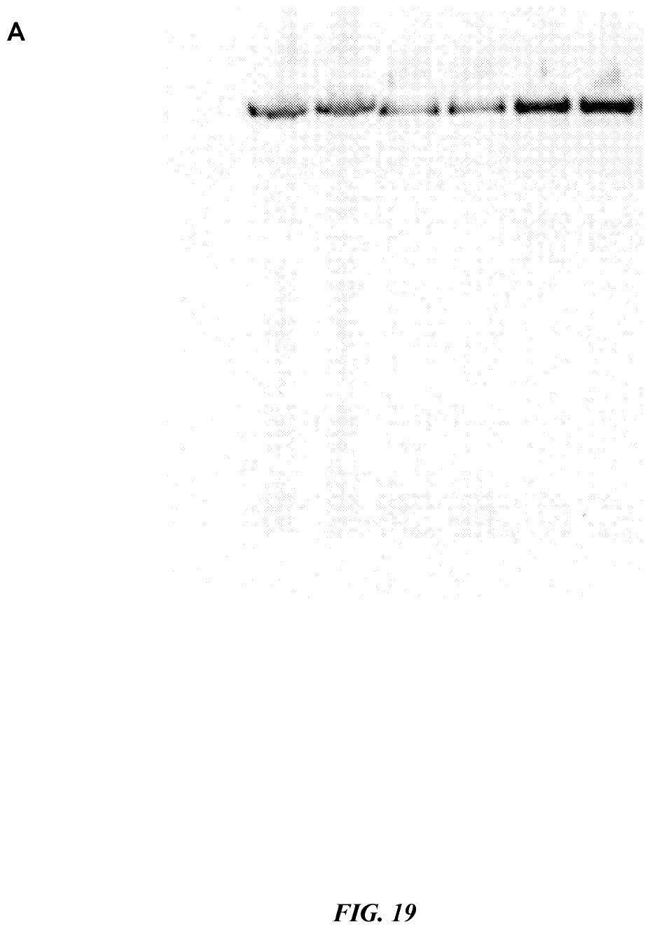

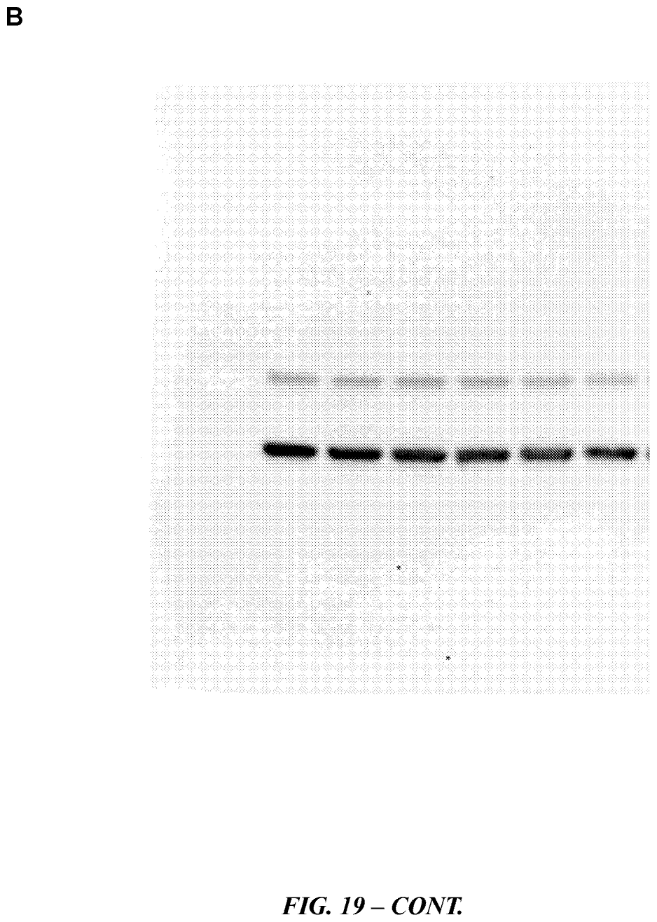

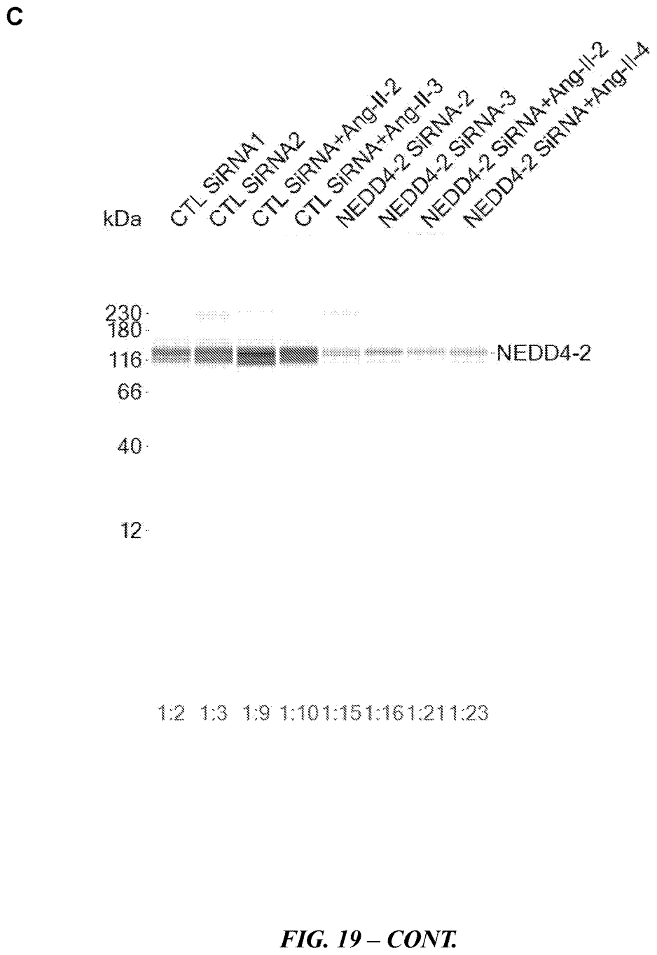

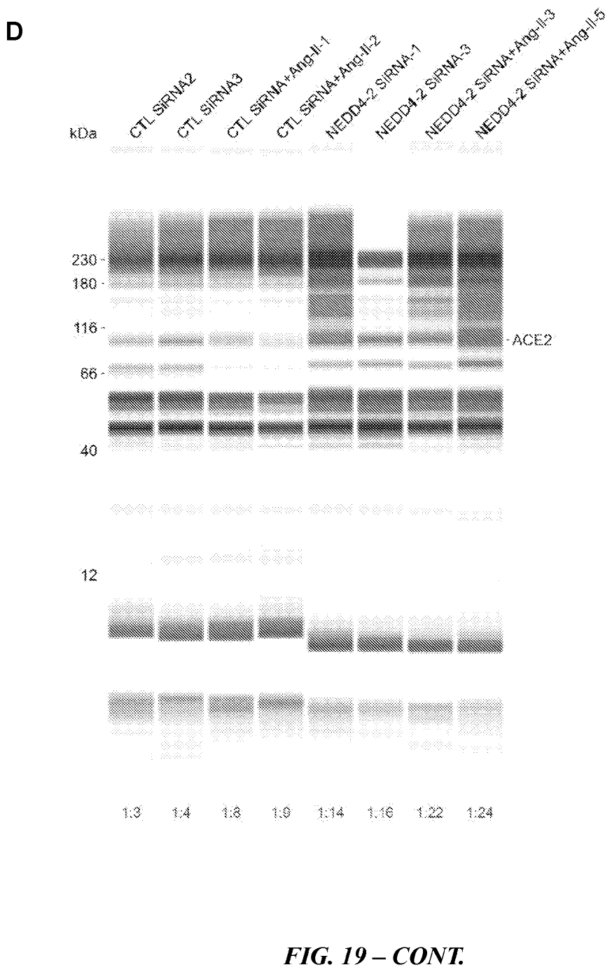

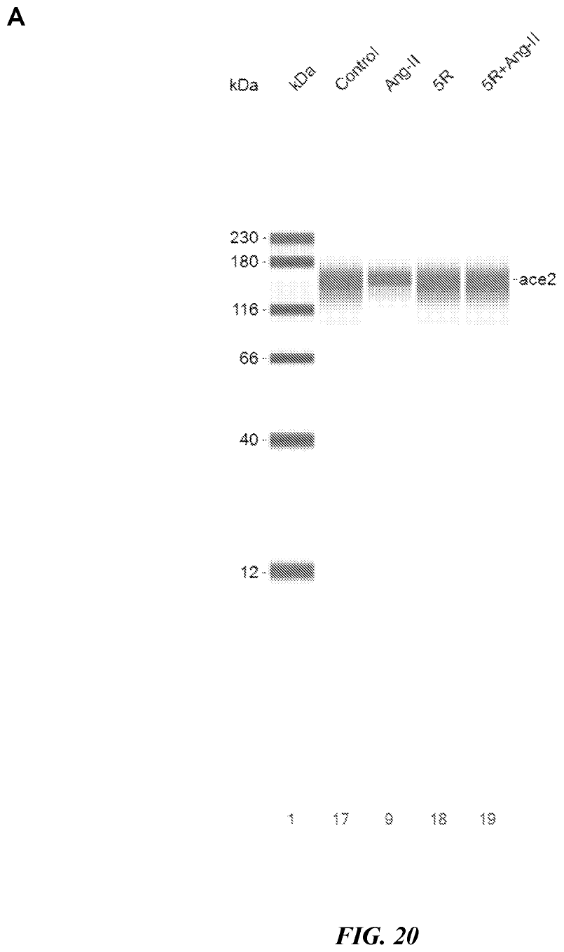

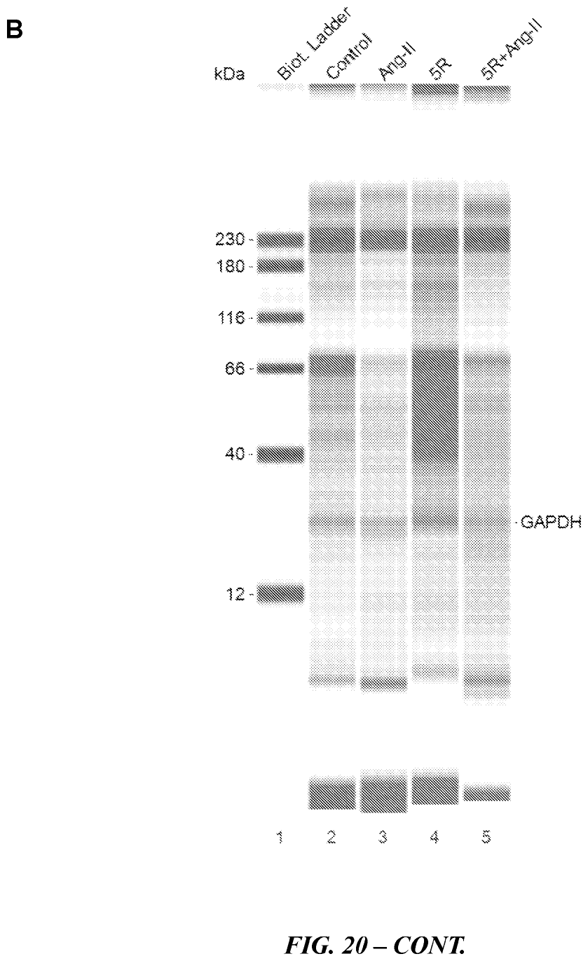

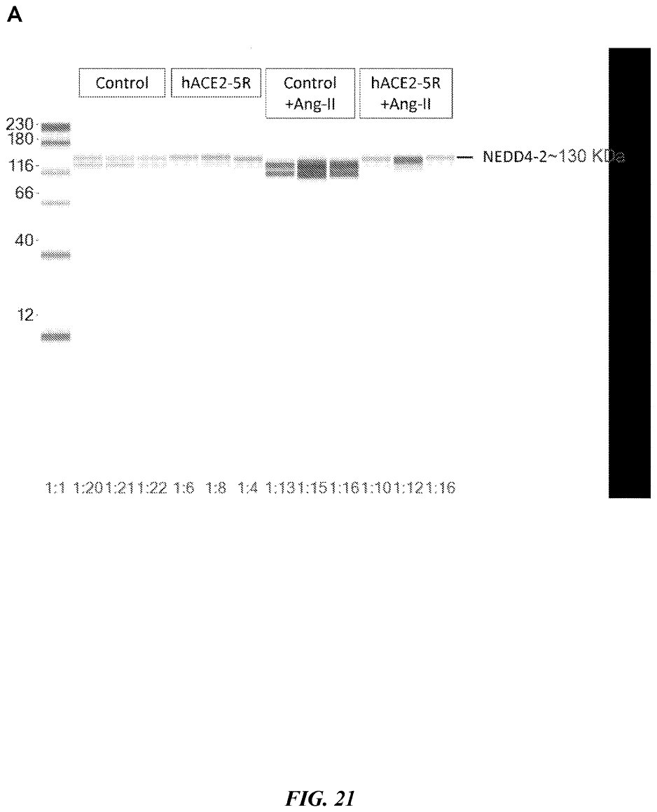

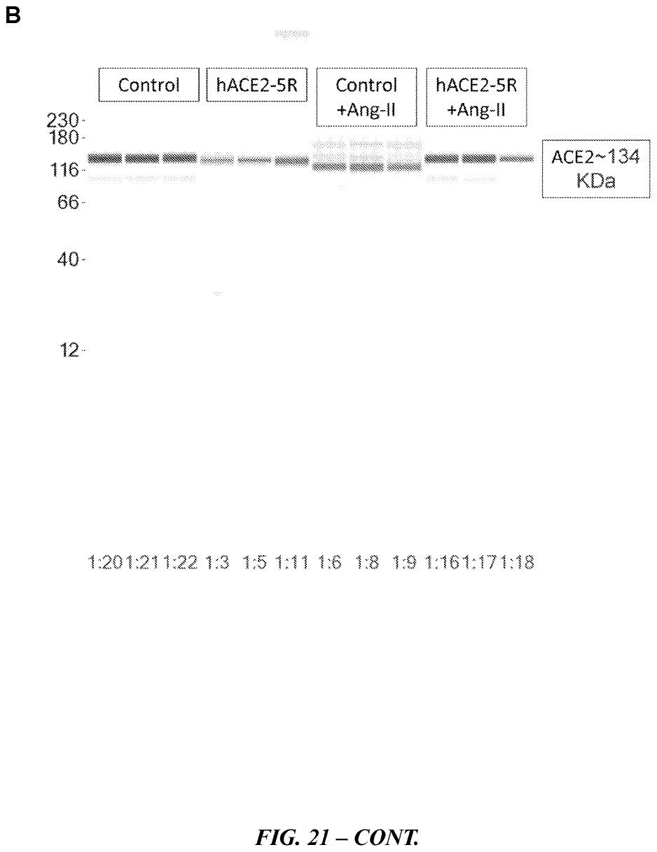

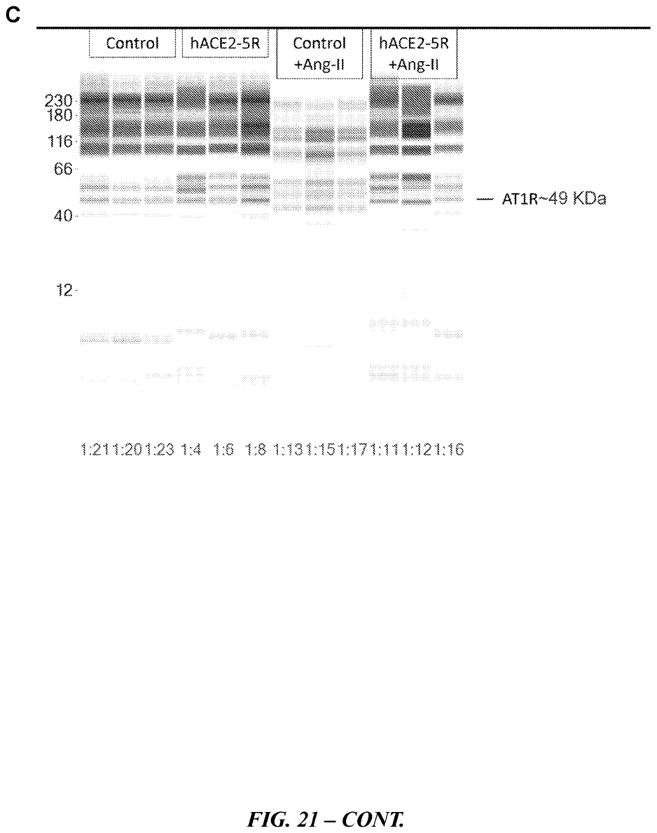

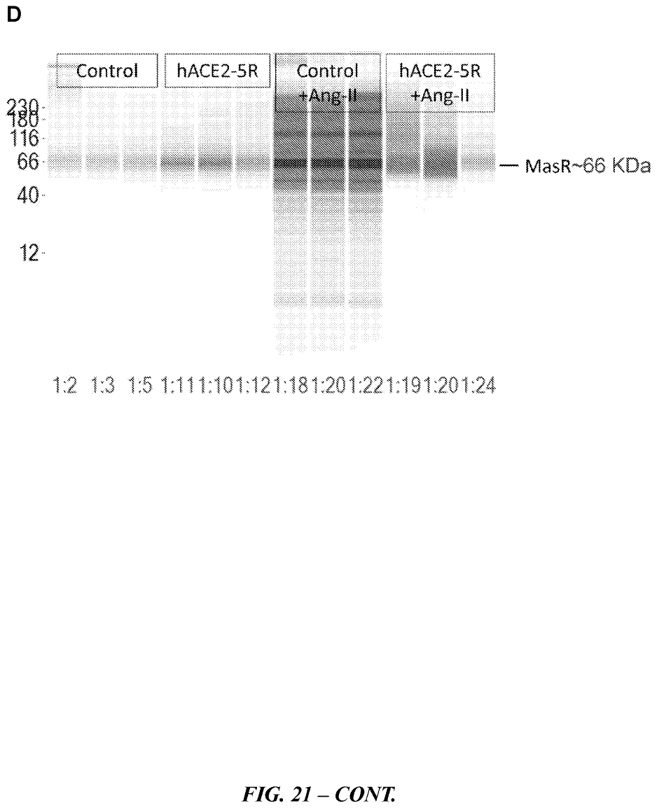

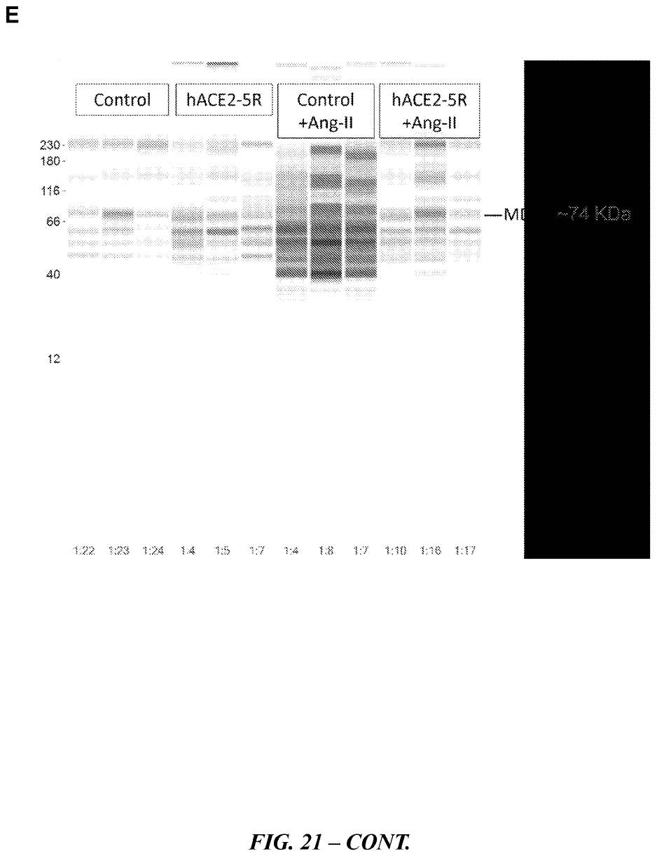

The next set of experiments aimed at establishing a direct relationship between ACE2 and Nedd4-2. Transfection of HEK293T cells with wild-type (WT) Nedd4-2 significantly decreased basal ACE2 activity, and this was not further reduced by Ang-II treatment (FIG. 3, panel A). In contrast, transfection with a catalytically inactive Nedd4-2 mutant with a serine mutated to cysteine (C/S) in the HECT domain of the protein 15had no effect on basal ACE2 activity. In addition, this catalytically inactive form prevented Ang-II-induced ACE2 down-regulation (FIG. 3, panel A). Overexpression of WT Nedd4-2 also reduced cellular ACE2 levels, which was not observed in cells transfected with the catalytically inactive Nedd4-2 mutant (FIG. 3, panel B). To further establish the direct relationship between ACE2 and Nedd4-2, independent of transfection, in non-cancerous cells, primary human aorta endothelial cells (HAEC) that naturally express both ACE2 and Nedd4-2 were used. As with HEK293T (FIG. 9) and Neuro2A cells, 4HAEC exposed to Ang-II exhibited ACE2 internalization and resulted in increased co-localization with intracellular Nedd4-2 (FIG. 3, panel C). In these cells, Ang-II-mediated up-regulation of Nedd4-2 was also associated with a reduction in ACE2 levels (FIG. 3, panel D). Pre-treatment with Nedd4-2 siRNA significantly knocked down the expression of the E3 ubiquitin ligase and resulted in a restoration of ACE2 levels that were no longer reduced by Ang-II treatment (FIG. 3D). Together, these data establish causality between Nedd4-2 up-regulation and ACE2 down-regulation, confirming that Nedd4-2 is required for Ang-II-mediated ACE2 ubiquitination.

### Ubiquitination-Resistant ACE2 Exhibits Enhanced Activity

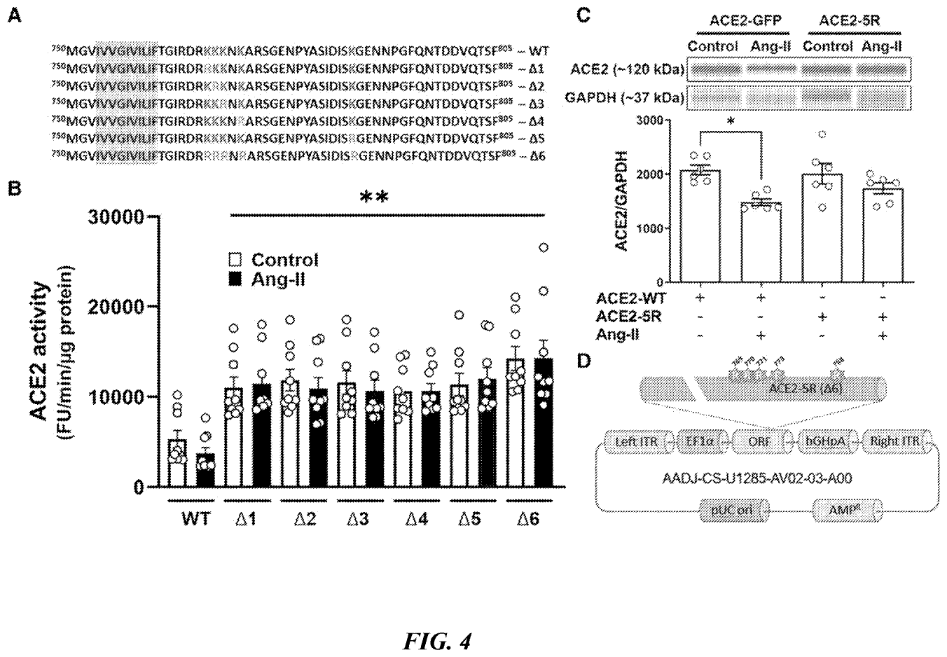

Since Nedd4-2 has 13 known and 2262 predicted high-confidence substrates in the mouse, including ACE, we focused our targeting strategy on ACE2 ubiquitination rather than Nedd4-2 itself. Based on the predicted interaction of Nedd4-2 with the ACE2 carboxy tail (FIG. 2, panel B), we next designed an ACE2 mutant resistant to Nedd4-2 ubiquitination. Ubiquitination takes place primarily at lysine sites embedded in the intracellular part of the protein.⁸-10 Human ACE2 has a short intracellular C-terminus consisting of 43 amino acids, among which five lysine residues constitute putative ubiquitination sites. These residues are also located next to one of the four predicted Nedd4-2 recognition motifs (FIG. 4, panel A). To determine the role of these lysine residues in the regulation of ACE2 expression levels and activity, we generated mutants in which lysine residues were substituted by arginine, another positively charged amino acid that cannot be ubiquitinated.¹⁶ The first five mutants had a single lysine mutated to arginine (FIG. 4, panel A), while the sixth mutant (46 or ACE2-5R) included all five mutations. Basal ACE2 activity in transfected HEK293T cells was significantly increased in all mutants (FIG. 4, panel B). ACE2-5R displayed an enzymatic activity comparable to other ACE2 mutants, indicating that lysine residues from the C-terminus play redundant roles in Ang-II-induced ACE2 ubiquitination. Furthermore, all mutants, including ACE2-5R, were resistant to Ang-II-mediated degradation (FIG. 4, panel B and C). Transfection with hACE2-5R resulted in a four-fold increase in ACE2 activity (FIG. 4, panel B), independent of any increase in ACE2 expression (FIG. 4, panel C), despite using an antibody targeting amino acids 392-744 that do not overlap with the point mutations. These data suggest that hACE2-5R not only is resistant to Ang-II-mediated ubiquitination but also exhibits enhanced activity compared to native ACE2. Accordingly, ACE2-5R was selected for in vivo targeting of ACE2 ubiquitination and packaged into a commercial adeno-associated viral delivery system (FIG. 4, panel D).

### ACE2 is Expressed on GABAergic Neurons

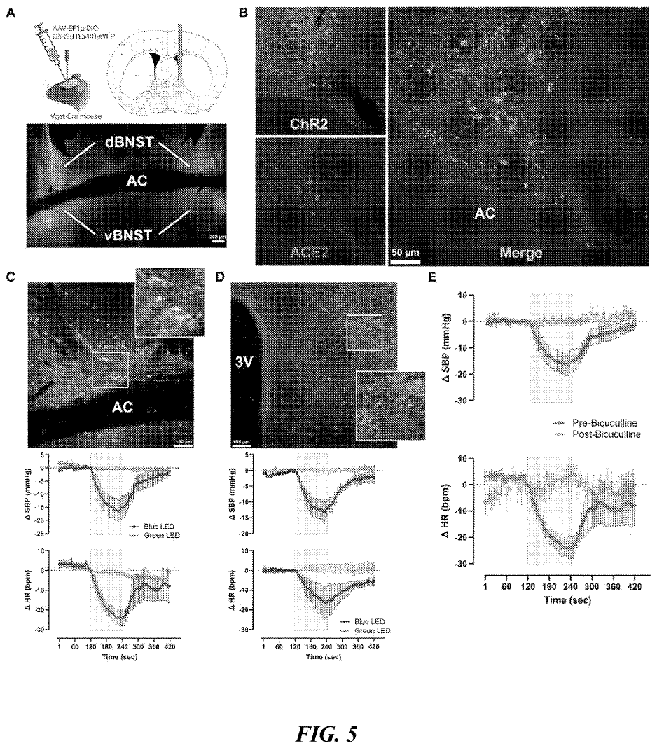

Having generated a new gene therapy tool, the next set of experiments aimed at validating the injection site for optimum targeting of ACE2 ubiquitination. Our previous work showed that ACE2 is expressed in the BNST and suggested that expression on GABAergic neurons in the BNST might contribute to an inhibitory input to PVN excitatory neurons. 12,14To validate the BNST as an appropriate region for ACE2-5R injection, we first verified that (i) GABAergic neurons in the BNST are expressing ACE2 and (ii) these GABAergic neurons project an inhibitory tone to the PVN. Injection of a cre-dependent AAV-ChR2-eYFP in the BNST of Vgat-cre mice (FIG. 5, panel A) resulted in the expression of eYFP fluorescence selectively on GABAergic neurons, and ACE2 co-localization confirmed the enzyme expression on these inhibitory neurons (FIG. 5, panel B). Importantly, ACE2 immunoreactivity was only detected on GABAergic cell bodies and was undetectable on the outer edge of the BNST where GABAergic neurons are absent. To verify that these GABAergic neurons are involved in blood pressure (BP) regulation, unilateral photoactivation of neuronal cell bodies in the dorsal BNST (FIG. 5, panel C) was performed in anesthetized Vgat-cre mice using a blue LED light (473 nm, 1-10 Hz, 1 min). This resulted in a time- and frequency-dependent reduction in both mean BP (~16 mmHg) and heart rate (HR) (~25 b.p.m.) that immediately returned to baseline once the stimulation ceased while shining a green LED light (532 nm) over the dorsal BNST produced no response. To further confirm that these GABAergic neurons regulate BP via inhibition of PVN neurons, the same protocol was used to stimulate GABAergic projections to the PVN (FIG. 5, panel D). The blue light produced a similar reduction in mean BP and HR following direct stimulation of the BNST. Importantly, the timeframe of these responses suggests an autonomic rather than a hormonal pathway. Finally, to verify that these GABAergic inhibitory projections terminate in the PVN, photostimulations of BNST GABA neurons were repeated following the blockade of GABAA receptors in the PVN. Bicuculline injections into the PVN (FIG. 5, panel E) prevented the reduction in BP and HR. Together, these data confirm that ACE2 is expressed on GABAergic neurons in the BNST and that these neurons exert an inhibitory tone to PVN excitatory neurons involved in BP regulation. This set of experiments confirms that the BNST is an appropriate target region to further study ACE2 ubiquitination and its role in neurogenic hypertension.

### ACE2-5R Enhances the GABAergic Inhibitory Tone

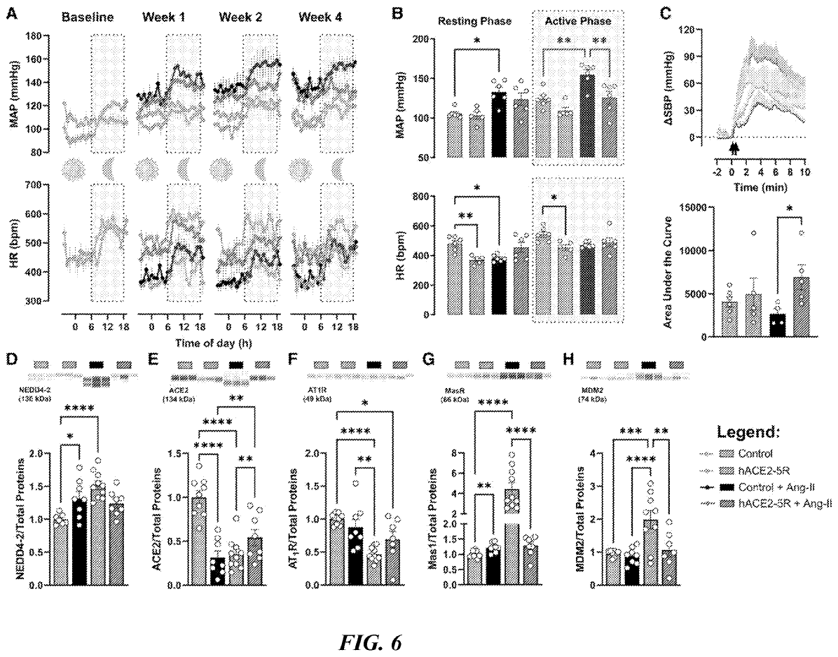

To assess the impact of ACE2 ubiquitination in the BNST in the development of neurogenic hypertension, C57BL6/J male mice were injected bilaterally with AAV-hACE2-5R or AAV-GCaMP-6S as the control virus. Six weeks were allowed for viral expression before chronic infusion with Ang-II to induce neurogenic hypertension. In normotensive mice, hACE2-5R expression resulted in a significant reduction in baseline mean BP during the resting phase (93±3 vs. 108±4 mmHg, P<0.05) but not the active phase (107±4 vs. 117±3 mmHg, P=0.08) of the nychthemeral cycle, without affecting HR (FIG. 6, panel A). The lack of HR changes contrasts with the optogenetic data showing pronounced bradycardia resulting from a forced stimulation of BNST GABAergic neurons devoid of any regulatory influence (FIG. 5, panel C). In conscious mice, however, baroreflex regulation is likely to have buffered this bradycardia. The reduction in BP in the hACE2-5R group did not persist, and only bradycardia was observed in both active and resting normotensive mice (without Ang-II infusion) by the end of the infusion (FIG. 6, panels A and B). Ang-II infusion resulted in a significant rise in mean BP, associated with bradycardia. Expression of hACE2-5R in Ang-II-infused mice resulted in a reduction in BP, as early as the second week of infusion, which was restricted to the active phase and abolished the circadian fluctuations of BP. To determine the impact of ACE2 ubiquitination on the GABAergic inhibitory tone to the PVN, BP was recorded in anaesthetized mice following blockade of GABAA receptors in the PVN. Removal of GABAergic input to the PVN following bilateral bicuculline injections resulted in an immediate rise in BP that was exacerbated in hACE2-5R mice infused with Ang-II (FIG. 6, panel C). This suggests that prevention of ACE2 ubiquitination in the BNST resulted in enhanced GABAergic input to the PVN, capable of blunting the development of hypertension. Analysis of protein expression in the BNST confirmed that Ang-II infusion was associated with increased Nedd4-2 (FIG. 6, panel D) and a concomitant reduction in ACE2 levels (FIG. 6, panel E), while AT₁R, Mas1 receptor (Mas1R), and MDM2 were not changed. Surprisingly, hACE2-5R injection in normotensive mice resulted in a reduction in ACE2. Although this was concomitant to Nedd4-2 (FIG. 6, panel D) and MDM2 (FIG. 6, panel H) up-regulation, these E3 ligases are unlikely responsible since their target lysine residues were mutated. This was also associated with a down-regulation of AT1R and an up-regulation of Mas 1R, suggesting that hACE2-5R enhanced activity had effectively compensated for the reduction in ACE2 expression. In hypertensive mice, although Nedd4-2 up-regulation was not significantly affected by hACE2-5R, ACE2 was significantly increased and AT1R was reduced (FIG. 6, panel F), while Mas1R (FIG. 6, panel G) expression was not affected and MDM2 was similar to baseline levels (FIG. 6, panel H). These data suggest that, unlike ACE2, hACE2-5R is resistant to hypothalamic E3 ligases and able to blunt the development of neurogenic hypertension by enhanced processing of Ang-II levels.

### DISCUSSION

Although impaired ACE2 activity has been extensively shown to be associated with cardiovascular diseases, including hypertension, the precise mechanisms responsible remain largely unknown.²⁻⁴ In this study, we identify Nedd4-2 as a mediator of ACE2 ubiquitination and we propose a novel cellular mechanism behind the development of neurogenic hypertension. Specifically, we demonstrate that Ang-II mediates Nedd4-2 up-regulation in experimental hypertension, which in turn promotes ACE2 ubiquitination and degradation. Nedd4-2 up-regulation was also observed in hypertensive patients. Knockdown of Nedd4-2 expression or activity prevented Ang-II-mediated down-regulation of ACE2. Finally, we validated these findings in a neurogenic hypertension model and showed that expression of an ACE2 ubiquitination-resistant mutant resulted in an enhanced inhibitory input to pre-sympathetic PVN neurons and a reduction in the development of hypertension.

The activity of any specific protein, including ACE2, is critically dependent on cellular expression levels and its subcellular localization. Since its discovery two decades ago, many important findings have highlighted the multifunctional role of ACE2 within and outside the RAS. Indeed, ACE2 has been shown to modulate circulating levels of Ang-II and Ang-(1-7), which play crucial and opposing roles in the regulation of cardiovascular function.¹⁷ The recent discovery that ACE2 is a cellular receptor for SARS-COV-2, the coronavirus responsible for COVID-19 disease, has exponentially increased research aimed at understanding the mechanisms controlling its expression levels and subcellular localization. Both Ang-II and SARS-COV-2 have been shown to induce AT1R-dependent internalization of plasma membrane ACE2 followed by a decrease in its total cellular levels.^{4,5} Based on our initial findings that ACE2 is ubiquitinated in basal conditions,⁴ we hypothesized that this post-translational modification is responsible for lysosomal targeting of the enzyme in neurogenic hypertension. However, only a few studies have attempted to identify E3 ligases involved in ACE2 ubiquitination, and none have investigated this interaction in neurogenic hypertension. Our combined proteomic and bioinformatic approach highlighted Nedd4-2 as a highly likely E3 ligase participating in ACE2 ubiquitination, with four potential recognition sites, including one close to the C-terminal of ACE2 in a lysine-rich region.

Nedd4-2 belongs to the HECT-containing subfamily of E3 ligases and is involved in the regulation of cell proliferation, viral budding, and intracellular trafficking. These ligases modulate G protein-coupled receptor activity either through direct interaction or via binding to β-arrestin.^{15,16} As ubiquitination takes place exclusively at intracellular lysine residues, we examined the five such residues localized in the intracellular C-terminus of ACE2, next to the Nedd4-2 recognition motif (FIG. 4). Mutation of any of these lysine residues led to enhanced enzymatic activity, clearly demonstrating that ACE2 is constitutively ubiquitinated and supporting the potential therapeutic benefit of using ubiquitination-resistant ACE2 to overcome hyperactivity of the RAS.

Nedd4-2 is a well-known E3 ubiquitin-protein ligase controlling cell surface expression of kidney epithelial Na⁺ channels (ENaC), and impaired function of Nedd4-2 is associated with salt-sensitive hypertension. 18,19 Our data highlight another mechanism by which Nedd4-2 may contribute to alterations in cardiovascular function, namely by decreasing ACE2 expression levels and activity. This effect requires the enzymatic activity of Nedd4-2 as its catalytically inactive mutant¹⁵ had no effect on Ang-II-induced ACE2 down-regulation (FIG. 3, panels A and B). Interestingly, Ang-II treatment enhanced co-localization between ACE2 and Nedd4-2, suggesting that AT₁R stimulation promotes ACE2 ubiquitination, at least partially, through Nedd4-2. Whether or not these effects are limited only to Nedd4-2 out of the large family of E3 ubiquitin ligases remains to be determined.

While ACE2 expression is known to be tissue-dependent and generally higher in males than females,²⁰ our findings highlight previously unreported sex differences in Nedd4-2 protein expression, with enhanced up-regulation in hypertensive males across tissues and limited up-regulation in females, restricted to the heart. These intriguing data suggest that females have signaling mechanisms that prevent Nedd4-2 up-regulation. This finding together with other supportive data we recently encountered is currently under investigation to further understand the crosstalk between sex hormones and Nedd4-2 expression in female mice. The sex-specific differences in Nedd4-2 expression are consistent with previous reports of Nedd4-2 gene polymorphisms associated with hypertension in males and the influence of oestrogens on reduced Nedd4-2 gene expression in the brain of females.^{21,22} Due to its role in ENaC ubiquitination, Nedd4-2 has been studied extensively in the context of salt-sensitive hypertension, which is thought to be more prevalent in females than males.²³ In addition, polymorphisms of Nedd4-2 have been associated with hypertension in African Americans and other ethnic groups.²⁴⁻²⁶ Accordingly, it is thought that mutation or a lack of Nedd4-2 results in elevated BP due to reduced ENaC ubiquitination. Our study differs from these observations. Indeed, Ang-II-mediated hypertension, which is associated with salt retention, was unequivocally associated with Nedd4-2 up-regulation (FIGS. 2 and 6, panel D), consistent with the increase also observed in the nodose ganglion of spontaneously hypertensive rats.²⁷ This up-regulation paralleled a reduction in ACE2 levels in male mice (FIGS. 2, panels D-F and 6, panel E) and African Americans (FIG. 2, panel G). While our in vitro knockdown experiments established causality between high Nedd4-2 and low ACE2 expression (FIG. 3), it is unclear at this time why these findings did not extend to Caucasians (FIG. 9). Although African Americans are more susceptible to salt-sensitive hypertension,^{24,25} it is likely that the patients' medications, notably those interfering with the RAS, also contributed to this observation. Unfortunately, the limited clinical information available from these donors did not allow for more in-depth analysis, and further studies are warranted in a larger patient population to investigate whether the up-regulation of Nedd4-2 selectively in African Americans was related to ethnicity or dependent on prescribed medications.

We previously reported that ACE2 expression and activity are impaired in the brain during the development of neurogenic hypertension^{3,28} and hypothesized that ACE2 ubiquitination could contribute to this down-regulation. The BNST is a neurochemically heterogeneous region, also known as the ‘extended amygdala’, which plays a role in cardiovascular responses to stress.¹³ Most GABAergic neurons project to the parvocellular cluster of the PVN and provide an inhibitory tone to glutamatergic pre-sympathetic neurons. In addition, increased neuronal activity in the PVN has been reported following lesions of GABAergic cells within the anterior BNST,²⁹ suggesting that the BNST could differentially modulate autonomic responses to stressors. While stress-related cardiovascular responses involving baroreflex mechanisms have been reported following pharmacological activation or inhibition of the BNST,^{13,30} the role of ACE2 ubiquitination in the BNST in the regulation of BP has never been investigated. Previous work from our group reported that ACE2 is expressed in the BNST and expression on GABAergic cell bodies could maintain an inhibitory input to PVN excitatory neurons, thus contributing to BP homeostasis.^{12,14} Our new observations confirm the expression of ACE2 on GABAergic BNST neurons and that selective photostimulations of these cells result in a frequency- and time-dependent reduction in BP and HR that can be prevented by selective blockade of GABAA receptors in the PVN, further confirming direct projection from the BNST to the PVN. Although we also previously showed expression of ACE2 on vasopressinergic neurons,¹² the immediate responses following optogenetic stimulations suggest the involvement of the autonomic nervous system rather than hormonal responses. Importantly, we show that transfection of these GABAergic neurons in the BNST with a ubiquitination-resistant ACE2 mutant resulted in a significant reduction in baseline BP. This effect was exacerbated in hypertensive mice, notably during the active phase. These data confirm that ACE2 is constitutively ubiquitinated within the BNST, which may adversely affect the regulation of BP at baseline and in hypertensive conditions. Since ACE2 is widely expressed in the brain, we speculate that other ACE2-expressing GABAergic neurons projecting to the PVN could also regulate various physiological functions via similar mechanisms, thus warranting further investigation.

E3 ubiquitin ligases have previously been reported to affect GABAergic neurotransmission, leading to the modification of adaptor proteins and secondary signaling pathways, resulting in an increased or decreased inhibitory tone.^{31,32} The BNST is also known to contain AT₁R, and Ang-II regulates neuronal activity in this region. 30,33,34 Consistent with our observations, activation of AT₁R in the BNST during Ang-II-mediated hypertension up-regulated Nedd4-2, similar to that observed in HEK293T and endothelial cells, which in turn reduced ACE2 expression on the cell surface and likely affected the conversion of Ang-II to Ang-(1-7). On the other hand, prevention of ubiquitination in the BNST would likely result in enhanced processing of Ang-II, either blood-borne or locally generated, and formation of Ang-(1-7). Hypertension was not associated with significant changes in AT₁R (FIG. 6, panel F). Although the BNST is thought to lack AT₂R,³⁵ it contains Mas1R,³⁶ both of which can bind Ang-(1-7), and the up-regulation of Mas1R could be explained by a lack of Ang-(1-7) as a result of ACE2 ubiquitination and degradation. Interestingly, the up-regulation of Nedd4-2 and MDM2 following hACE2-5R expression in normotensive mice suggests that ACE2 ubiquitination is a tightly regulated mechanism and that targeting individual E3 ligases might not produce therapeutic benefits as feedback mechanisms would attempt to compensate for individual knockdowns.

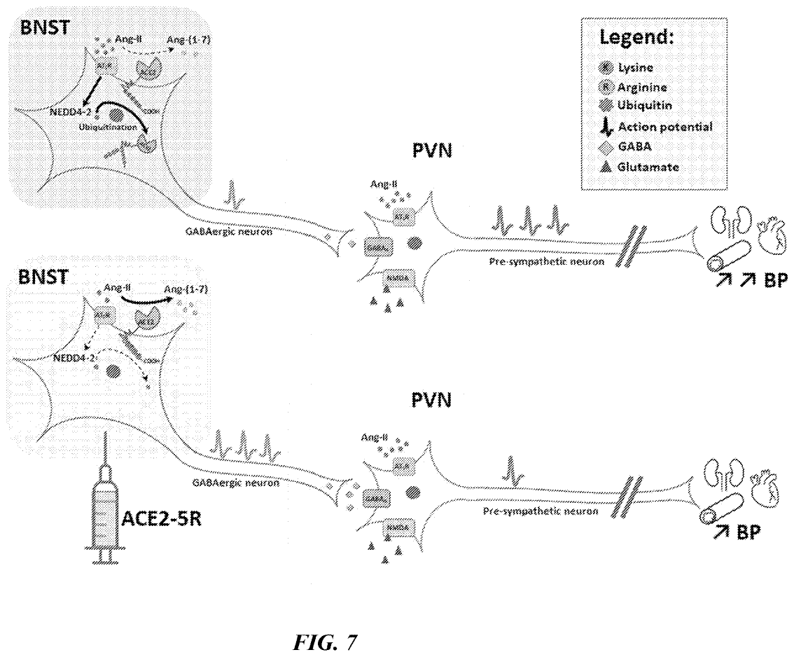

Based on current and previous observations, a working model was designed (FIG. 7) where Nedd4-2 could contribute to the excitatory activity of PVN neurons and downstream sympathetic activity in hypertension. Nedd4-2 has previously been shown to contribute to the activity of excitatory neurons, notably by affecting the expression of ion channels and cell surface proteins.³⁷ However, the role of Nedd4-2 in the brain is complex as it involves multiple and often opposite mechanisms. For example, a lack of Nedd4-2 ubiquitination, including in the brain, is known to be associated with an up-regulation of ENaC, resulting in salt-induced hypertension.³⁸ Conversely, Nedd4-2 knockdown also leads to reduced glutamate transporter ubiquitination and increased glutamate uptake,³⁹ possibly preventing glutamate neuroexcitability that is associated with reduced ACE2 activity.⁴⁰ Therefore, using recombinant hACE2-5R to control neurogenic hypertension might be a better therapeutic approach than interfering with Nedd4-2 expression.

There are some limitations associated with our study. First, our in vivo experiments did not specifically target Nedd4-2 in the brain but instead focused on the prevention of ACE2 ubiquitination in the BNST. Although this strategy ensured specificity regarding ACE2 ubiquitination, our data also support the possibility that in addition to Nedd4-2, the reduction in BP observed could be related to other E3 ligases targeting ACE2. However, while MDM2 and Skp2 have been reported to ubiquitinate ACE2,^{6,7} they have not been studied outside the lung. Our data show that MDM2, which was previously shown to ubiquitinate ACE2 at ⁷⁸⁸Lys, is not up-regulated by Ang-II infusion (FIG. 6, panel H), suggesting that it plays no part in our hypertension model. Second, although the reduction in ACE2 in the BNST appears to be compensated by the enhanced catalytic activity of ACE2-5R in neurogenic hypertension, the mechanism for this reduction in expression remains unknown. While ubiquitination is unlikely, ACE2 shedding³ by ADAM17 is a possibility, and more work is needed to confirm this hypothesis. At the same time, assessment of hACE2-5R catalytic activity in the brain should be further investigated, ideally in a model with broader expression to overcome detection limitations. Finally, it is unclear how the Ang-II-mediated up-regulation of Nedd4-2 may have affected other targets of this E3 ligase, such as ENaC and Piezo2, and whether these targets contributed to the development of hypertension in our model.

In conclusion, we identified Nedd4-2 as a new E3 ligase targeting ACE2 in neurogenic hypertension. Nedd4-2 is up-regulated by elevated Ang-II levels and promotes ACE2 ubiquitination and degradation. Ubiquitination of ACE2 on GABAergic neurons located in the BNST results in a dampening of the inhibitory input to pre-sympathetic neurons in the PVN, thus contributing to the development of hypertension. Prevention of its ubiquitination allowed for preserved ACE2 compensatory activity in the BNST, leading to enhanced Ang-II conversion to Ang-(1-7), reduced up-regulation of Nedd4-2, and an increased inhibitory tone to the PVN, resulting in a dampening of neurogenic hypertension.

### Translational Perspective

While angiotensin-converting enzyme 2 (ACE2) conversion of angiotensin (Ang)-II to Ang-(1-7) is supposed to limit the overactivation of the renin-angiotensin system (RAS), the enzyme is down-regulated during the development of hypertension. As antihypertensive RAS blockers on the market only provide limited control of blood pressure among hypertensive patients, understanding the mechanisms responsible for this blunted compensation provides new possible targets for the treatment of hypertension. In this study, we show that Nedd4-2 up-regulation is associated with ACE2 ubiquitination, while prevention of this post-translational modification prevents the development of hypertension. Accordingly, targeting of ACE2 ubiquitination provides a new treatment strategy to reduce hypertension.

### Methods

### Animals

Experiments were conducted in adult C57BL6/J and Vgat-IRES-cre mice (10-12 weeks old, 20-25 g; Jackson Laboratory, Bar Harbor, ME) from both sexes. Mice were housed in a temperature (~25° C.) and humidity-controlled facility under a reversed 12-hour dark/light cycle, fed standard mouse chow (Envigo, iOS Teklab Extruded Rodent Diet 2019S, Huntingdon, UK) and water ad libitum. All procedures conformed to the National Institutes of Health Guide for the Care and Use of Laboratory Animals and were approved by the Louisiana State University Health Sciences Center (#3540), and the Southeast Veterans Healthcare System (#620) Institutional Animal Care and Use Committees in accordance with the ‘Principles of Laboratory Animal Care by the National Society for Medical research and the Guide for the Care and Use of Laboratory Animals’ (National Institutes of Health Publication No. 86-23, revised 1996). Cardiac samples from patients were obtained from The Medical College of Wisconsin (IRB #PRO00010828) and Duke University (IRB #PRO00005621) from organs not suitable for transplant. Informed consents were obtained prior to experiments and all procedures conform to the principles outlined in the Declaration of Helsinki.

### Construction of ACE2 Mutants

All receptor mutants were obtained using the Quick-Change site-directed mutagenesis kit (Agilent, Santa Clara, CA). The following primers were used to obtain the various mutants (where the mutation is shown in bold italic characters): Mutant 1: Forward: 5′ atc aga gat cgg agg aag aaa aat aaa 3′, Reverse: 3′ ttt att ttt ctt ctt ccg atc tet gat 5′; Mutant 2: Forward: 5′ aga gat cgg aag agg aaa aat aaa gca 3′, Reverse: 3′ tgc ttt att ttt cct ctt ccg atc tct 5′; Mutant 3: Forward: 5′gat cgg aag aag aga aat aaa gca aga 3′, Reverse: 3′ tct tgc ttt att tct ctt ctt ccg atc 5′; Mutant 4: Forward: 5′ cgg aag aag aaa aat aga gca aga agt gga 3′, Reverse: 3′ tcc act tct tgc tct att ttt ctt ctt ccg 5′; Mutant 5: Forward: 5′ tcc atc gat att age aga gga gaa aat aat 3′, Reverse: 3′ att att ttc tcc tct gct aat atc gat gga 5′. The sequence of each construct was confirmed by restriction mapping and nucleotide sequence analysis (Integrated DNA Technologies, Coralville, IA).

### Cell Culture and Maintenance

Human embryonic kidney 293T cells (HEK293T; #CRL-3216, ATCC) were cultured in Dulbecco's Modified Eagle's Medium (DMEM) (#30-2002, ATCC), 10% fetal bovine serum (heat inactivated, #30-2020, ATCC), 2 mM L-glutamine (#30-2214, ATCC) as per the provider's instructions. Primary human aortic endothelial cells (HAEC; #PCS-100-011, ATCC) were cultured in endothelial cell growth kit-VEGF (#PCS-100-041, ATCC). Upon reaching ~90% confluency, the cells were trypsinized and seeded in six-well plates at a density of 3×105 cells per well in culture medium (2 mL) for 24 h. The cells were serum-starved 24 h before each experiment. Neuro2A cells (#CCL-131, ATCC) were seeded in 6 well plates with complete media including eagle's minimum essential medium (EMEM, #30-2003, ATCC), 10% Heat inactivated fetal bovine serum (#10082, Gibco), 1.5 g/L sodium bicarbonate (#25080, Gibco), 0.1 mM non-essential amino acids (Cat: 11140, Gibco), 1.0 mM sodium pyruvate (#11360, Gibco), 1% Pen-Strep (#15140, Gibco). After cells reached 80% confluence, cells were either treated with PYR-41 (50 μM for 2h, #N2915, Sigma Aldrich), PR-619 (20 μM for 6 h, #SML0430, Sigma Aldrich) or Ang-II (100 nM for 4 h, #A9525, Sigma Aldrich). Cells were then harvested in cell lysis buffer for protein quantification.

### Plasmid Transfection

HEK293T cells were seeded in 6 well plates and transfected with the following plasmids: AT₁R (gift from Dr. Guangyu Wu, Medical College of Georgia), hACE2-GFP (RG208442, Origene), HA-ubiquitin (#18712, Addgene), NEDD4-2 or catalytically inactive NEDD4-2 mutant (NEDD4-2 C/S, kind gift of Dr. A. Marchese, Medical College of Wisconsin) 15 and pcDNA3.1 (#114194, Addgene), using lipofectamine 2000 reagent (#11668019, Thermo Fisher), in DMEM with no antibiotics, at ~80% confluence, according to the manufacturer's instructions. After 6 h, the transfection medium was replaced with complete medium for 24 h and subsequently the cells were starved in OptiMEM (#11058021, Thermo Fisher) without phenol red and FBS for 24 h. The cells were then exposed to Ang-II (100 nM) or medium (control) for 4 h, after which the cells were harvested for assessment of ACE2 enzyme activity and ACE2 protein level. A similar protocol was used in another set of cells for immunohistochemistry.

siRNA Transfection

HAEC (#CC-2535, Lonza) were seeded into a 6 well plate at 60-80% confluency in reduced serum media (Opti-MEM, #31985062, Gibco) for 24 h. NEDD4-2 siRNA (10 μM, #s23571, Invitrogen) or a negative control siRNA (10 μM, #ASO2J03U, Invitrogen), and 3 μl lipofectamine RNAiMAX transfection reagent complexes were incubated for 5 min at room temperature (RT), added to the cells and left for 24 h. Ang-II (100 nM for 4 h, #A9525, Sigma Aldrich) was then added to the cells before harvesting, protein extraction and quantification.

### Western Blotting and Co-Immunoprecipitation

The cell lysates were collected in RIPA buffer and protein concentration was assessed using the Bradford method before being resuspended in loading buffer: 4× Laemmli buffer (4% SDS, 10% 2-mercaptoethanol, 20% glycerol, 0.004% bromophenol blue, 0.125 M Tris-HCl). The samples were separated by SDS-PAGE and transferred onto polyvinylidene difluoride membranes. The membranes were probed with antibodies against ACE2 (1:500, Santa Cruz #sc-73668), NEDD4-2 (Thermo Fisher Scientific, #2F6H6) and β-actin (Santa Cruz, #sc-8432). Primary antibodies were used at 1:500 dilutions and HRP-conjugated secondary antibodies (Cell Signaling) were used at 1:10,000 dilutions. The signal was detected using ECL Plus (Thermo Fisher Scientific).

Immunoprecipitation was performed as previously.⁴ In brief, HEK293T cells were cultured on 10 cm² dishes and transfected at ~80% confluency with 6 μg of GFP-tagged ACE2 for 48 h, 6 μg HA-ubiquitin and 6 μg AT₁R plasmids, fetal bovine serum (FBS) being omitted during the last 24 h. Cells were washed twice with phosphate-buffered saline and lysed in 500 μl of lysis buffer containing 50 mM Tris-HCl pH 7.4, 150 mM NaCl, 1% NP-40, 0.5% sodium deoxycholate, 0.1% SDS, and complete mini protease cocktail (Roche). After gentle rotation for 1 h, the samples were centrifuged for 15 min at 14,000g and the supernatant was incubated with 50 μl of protein A/G sepharose (#sc-2003, Santa Cruz Biotechnology) for 1 h at 4° C. to remove nonspecific bound proteins. Samples were then incubated with 5 μg of anti-GFP antibodies (#sc-9996, Santa Cruz) overnight at 4° C. with gentle rotation followed by incubation with 50 μl of protein A/G sepharose beads for 5 h. Resin was collected by centrifugation and washed four times with 500 μl of lysis buffer. Immunoprecipitated proteins were eluted with 100 μl of 1X SDS-PAGE loading buffer, separated by 10% SDS-PAGE and visualized by immunoblotting using HA-antibody (#A01621, GenScript).

### Immunohistochemistry

HEK293T and HAEC were seeded on poly-L-lysine-coated cover slips placed in 6 well plates. After 24 h, the cells were rinsed with 1×PBS, and fixed with 4% paraformaldehyde at RT for 15 min. The cells were washed twice with 1× glycine PBS (250 mM Glycine in 1×PBS) at RT for 15 min. Further, the cells were treated with permeabilization buffer (20 μL X-100 triton in 10 mL 1×PBS) at RT for 15 min then washed with 1×PBS at RT for 15 min. To prevent non-specific binding of antibodies or other reagents to the cells, blocking was carried out using 10% normal horse serum+0.3% triton X-100 at RT for 30 min. The blocking solution was replaced with primary antibody (ACE2, NEDD4-2) diluted to 1:500 in 2% normal horse serum+0.3% triton X-100 at 4° C. overnight. The cells were further washed three times with PBS at RT for 10 min. The cells were treated with respective secondary antibodies at 1:1000 dilution in 2% normal horse serum+0.3% Triton X-100 at RT for 1 h. Followed by washing in PBS for three times at RT for 10 min and the cover slips were mounted on slides using mounting media containing DAPI (Fluoromount-G mounting medium, #00-4959-52, Invitrogen). The imaging was carried out using confocal microscopy (Nikon AIR, NIS-Element's software).

For brain immunohistochemistry, mice were anesthetized with avertin (2,2,2-Tribromoethanol, 200 μg/g of body weight) prior to euthanasia. Avertin was chosen over ketamine/xylazine to avoid excessive bradycardia and ensure better tissue perfusion. Once the depth of anesthesia was confirmed (lack of reflex to pinch toe), mice were transcardially perfused with 4% paraformaldehyde in PBS. The brain was collected and immersed in 4% paraformaldehyde for 1 h at 4° C. then transferred to 30% sucrose at 4° C. until processed. Coronal sections were cut at 30 μm and mounted on glass slides before being exposed to blocking buffer (2% normal horse serum). Sections were incubated overnight with primary antibodies at 4° C.; goat anti-hACE2 (1:500, #AF93, R&Dsystems) followed by a donkey anti-goat Alexa Fluor 488 (1:500, #A-11055, Thermo Fisher Scientific) secondary antibody for 4 h. Immuno-stained sections were visualized using confocal microscopy (Nikon A1R) and images were captured at different magnifications.

### ACE2 Activity Assay

HEK293T cells and tissue samples were processed for ACE2 activity, as reported previously. 16 Briefly, the cells or ground-up tissue were suspended in 0.5% Triton X-100 in ACE2 reaction buffer containing 1 M NaCl, 0.5 mM ZnCl2, 75 mM Tris·HCl and 100 μM Mca-YVADAPK(Dnp). Fluorescence emission at 405 nm, after excitation at 320 nm, was measured and the slope of fluorescence development between 10 and 120 min of incubation was calculated. Data are presented as fluorescence units (amount of substrate converted to fluorescent product) per minute normalized for protein content (FU/min/ug).

### Capillary Western

Total proteins from tissues were extracted using a RIPA lysis buffer (#89901, Thermo Fisher Scientific, IL) containing protease inhibitor cocktail tablets (#04693159001, Roche) while cells were extracted using a cell lysis buffer (#895347, R&D Systems) containing proteases and phosphatases inhibitors and quantified using BCA protein assay kit (#23225, Thermo Fisher Scientific). Equal amounts of proteins were loaded and analyzed on a capillary-based immunoassay platform (ProteinSimple, San Jose, CA) as per the manufacturer's instructions using a 12-240 kDa separation module (#SM-W004, Bio-Techne R&D Systems). The samples were diluted to 0.5 μg/μl concentration in the sample buffer, incubated with a fluorescent master mix (#PS-ST01EZ, ProteinSimple) and heated at 95° C. for 5 min. The biotinylated protein ladder (#PS-ST01EZ, Protein Simple), samples, blocking reagent (#042-203, ProteinSimple), primary antibodies: ACE2 (#sc-390581, Santa Cruz Biotechnology; #ab239924, Abcam, 1:50, #21115-1-AP, Thermo Fisher for mouse tissues and #ab15348, Abcam for HAEC), GAPDH (#ab-9483, Abcam, 1:50), Mas1 receptor (#ab235914, Abcam, 1:50) and NEDD-2 (#ab245522, Abcam, 1:100), AT₁R (#PA5-20812, Invitrogen, 1:50) and MDM2 (#700555, Invitrogen, 1:10) were used. HRP-conjugated secondary antibodies (#042-205, #042-206, and #043-522-2, ProteinSimple) and the luminol-peroxide mixture (#043-311 and #043-379, ProteinSimple) as chemiluminescent substrate were added. The plate and cartridges were loaded into the Wes/Jess units with the following setting: stacking and separation at 475 V for 30 min; blocking reagent for 5 min; primary and secondary antibody both for 30 min. ACE2, Mas, MDM2 and AT₁R proteins were normalized to total protein using replex and total protein detection modules (#RP-001 and #DM-TP01, ProteinSimple). Compass software was used to process and analyze the results.

### Bioinformatic Analysis and Proteomics

The E3 ubiquitin ligases predictive bioinformatic analysis was performed using UbiBrowser 1.0 (http://ubibrowser.bio-it.cn/ubibrowser/).¹⁷

Discovery-based proteomics using tandem mass tags (TMTpro) and liquid chromatography mass spectrometry: samples were prepared for discovery-based quantitative proteomic analysis by the addition of SDS to 1% and sonicated until completely homogenous. The protein concentration was determined using BCA protein assay kit using an eight-point standard curve. Protein sample was prepared for trypsin digestion by reducing the cysteines with tris(2-carboxyethyl) phosphine followed by alkylation with Iodoacetamide. After Chloroform-Methanol precipitation, each protein pellet was digested with 1 μg trypsin overnight at 37° C. The digested product was labeled using 2-TMTpro 16 plex reagents Sets (Thermo Fisher Scientific), utilizing the 126 isotopologue as the common pooled internal control, according to the manufacturer's protocol.

An equal amount of each TMTpro-labelled sample was pooled together in a single tube and SepPak purified (Waters, Ireland) using acidic reverse phase conditions to remove unreacted TMTpro and quenched-TMTpro molecules. After drying to completion, an off-line fractionation step was employed to reduce the complexity of the sample using basic pH reverse phase chromatography (Dionex U3000, Thermo Fisher Scientific), as described previously. 18 Each fraction was subjected to a 95 min chromatographic method employing a gradient from 2-25% ACN in 0.1% formic acid (ACN/FA) over the course of 65 min, a gradient to 50% ACN/FA for an additional 10 min, a step to 90% ACN/FA for 5 min, and a 15 min re-equilibration into 2% ACN/FA. Chromatography was carried out in a “trap-and-load” format using an EASY-Spray source (Thermo Fisher Scientific); trap column C18 PepMap 100, 5 μm, 100 A and the separation column was an EASY-Spray PepMap RSLC C18 2 μm, 100A, 75 μm×25 cm (Thermo Fisher Scientific). The entire run was at a flow rate of 0.3 μl/min. Electrospray was achieved at 1.8 kV. TMTpro data acquisition utilized an MS3 approach for data collection, as previously described.¹⁹ The protein FASTA database was Mus musculus, SwissProt tax ID=10090, version 2017 Oct. 25 and contained 25,097 sequences. Static modifications included TMTpro reagents on lysine and N-terminus (+304.207), carbamidomethyl on cysteines (+57.021), dynamic phosphorylation of serine, threonine and tyrosine (+79.966 Da), and dynamic modification of oxidation of methionine (+15.9949). The mass spectrometry proteomics data has been deposited to the ProteomeXchange Consortium via the PRIDE partner repository with the dataset identifier PXD (PXD027183).20

### Adeno-Associated Viral Vectors Stereotaxic Injections

Under 2% isoflurane anesthesia and buprenorphine analgesia (0.12 mg/kg subcutaneously, once per day for 3 days), Vgat-IRES-cre mice were injected stereotaxically with a cre-dependent humanized ChR2 with H134R mutation fused to eYFP (pAAV1-EF1α-double floxed-ChR2 (H134R)-eYFP-WPRE-HGHpA, 100 nL, 7×10¹² vg/mL, catalog #20298-AAV1, Addgene), bilaterally into the BNST, (relative to bregma, AP: 0.14 mm, ML: ±0.8 mm, DV: −4.3 mm) using a pneumatic Picospritzer (WPI Inc.), as described previously.²¹

The mutant 6 human ACE2 (hACE2-5R) construct was packaged in an adeno-associated viral vector (5×10¹² GC/mL, #CS-U1285-AV02, AAV Prime Genecopoeia) under the control of a broad scope EF1α promoter (FIG. 4D). Prior to injection in the BNST, AAV-hACE2-5R was mixed with AAV-GCaMP-6S in a 4:1 ratio for postmortem tracking of the injection site. The mixture was injected bilaterally in the BNST as described above. Six weeks after virus injection the mice were subjected to acute or chronic blood pressure (BP) recording protocols. Upon completion of the experiments, ChR2-expressing mice were perfused with 4% PFA, as above, and the brains were sectioned in a cryostat. Sections (30 μm) were observed under a fluorescent microscope (Nikon AIR, NIS-Element's software) to verify that the injection successfully targeted the BNST. Only subjects with GFP fluorescence overlapping with the BNST (FIG. 5A) were included in the analysis.

For GCaMP6S-injected mice, the BNST was manually dissected for protein extraction from 4 consecutive sections of 250 μm (relative to bregma, AP: 0.74 to −0.22 mm). Only homogenates exhibiting a fluorescence signal were included.

### Optogenetic Photoactivation

Wireless LED optic fiber posts (Ø 250 μm, Amuza) were positioned 0.5 mm above the dorsal BNST (relative to bregma, AP: 0.14 mm, ML: ±0.8 mm, DV: −3.8 mm) 3 weeks after AAV-ChR2 virus injection. Anesthetized mice (Isoflurane 2%) were subjected to continuous BP recording during which optogenetic stimulation with a blue or green laser was used at 1, 5 and 10 Hz for 1 min (20 msec pulses; 10 mW).

### Acute BP Recording

On the day of experiment, mice were anesthetized with 2% isoflurane and a pressure catheter (#SPR-1000, Millar Mikro-Tip, ADInstruments) was inserted into the left common carotid artery and pushed towards the aorta. Pulsatile BP and heart rate (HR) signals were recorded using PowerLab data acquisition system and LabChart8 pro software (ADInstrument, Australia). Mice were then positioned on a stereotaxic frame (Stoelting) for PVN injections (Relative to bregma, AP: −0.5 mm, ML: −/+0.25 mm, DV: −4.75 mm), as described previously. 12 Bilateral microinjections of the GABAA receptor antagonist bicuculline methiodide (1 mM in 100 nL/site, Sigma) were made in the PVN using a 10 μL glass micro-pipette connected to a pneumatic Picospritzer (WPI Inc.). Photoactivation was performed 5 min after bicuculline injection, as described above.

### Chronic BP Recording and Ang-II Infusion

Before surgery, mice were anesthetized with isoflurane (2%) in an oxygen flow (1 L/minute) and placed on a heating pad to maintain body temperature around 37.5° C. Mice were implanted with telemetry probes (PA-C10 or HD-X10; DSI, St. Paul, MN) as previously.³ Following recovery, baseline BP was recorded for 24 hours in all groups.

Ang-II (450 ng/kg/min, #A9525, Sigma Aldrich) or vehicle (0.9% saline) infusion was performed by implanting subcutaneous osmotic mini pumps (#1004, Alzet) for a period of 4 weeks in two separate cohorts. After completion of chronic recordings, the mice were anesthetized with isoflurane 2% and euthanized by decapitation. The brain was then collected from each group (6 mice/group) and frozen over dry ice before BNST dissection as described above.

### Statistics

Data are presented as mean±SEM. Data were analyzed by Student's t-test, one-way or two-way ANOVA followed by post hoc comparisons, as appropriate, using Prism 9 (GraphPad Software, San Diego). Differences were considered statistically significant at P<0.05.

### References Cited in this Example

1. Chhabra K H, Chodavarapu H, Lazartigues E. Angiotensin converting enzyme 2: a new important player in the regulation of glycemia. IUBMB Life 2013; 65:731-738.
2. Xia H, Lazartigues E. Angiotensin-converting enzyme 2: central regulator for cardiovascular function. Curr Hypertens Rep 2010; 12:170-175.
3. Xia H, Sriramula S, Chhabra K, Lazartigues E. Brain ACE2 shedding contributes to the development of neurogenic hypertension. Circ Res 2013; 113:1087-1096.
4. Deshotels M R, Xia H, Sriramula S, Lazartigues E, Filipeanu C M. Angiotensin II mediates angiotensin converting enzyme type 2 internalization and degradation through an angiotensin II type I receptor-dependent mechanism. Hypertension 2014; 64:1368-1375.
5. Ogunlade B O, Lazartigues E, Filipeanu C M. Angiotensin type 1 receptor-dependent internalization of SARS-COV-2 by angiotensin-converting enzyme 2. Hypertension 2021; 77: e42-e43.
6. Shen H, Zhang J, Wang C, Jain P P, Xiong M, Shi X, Lei Y, Chen S, Yin Q, Thistlethwaite P A, Wang J, Gong K, Yuan Z Y, Yuan J X, Shyy J Y. MDM2-mediated ubiquitination of ACE2 contributes to the development of pulmonary arterial hypertension. Circulation 2020; 142:1190-1204.
7. Wang G, Zhao Q, Zhang H, Liang F, Zhang C, Wang J, Chen Z, Wu R, Yu H, Sun B, Guo H, Feng R, Xu K, Zhou G. Degradation of SARS-COV-2 receptor ACE2 by the E3 ubiquitin ligase Skp2 in lung epithelial cells. Front Med 2021; 15:252-263.
8. Hurley J H, Stenmark H. Molecular mechanisms of ubiquitin-dependent membrane traffic. Annu Rev Biophys 2011; 40:119-142.
9. Komander D. The emerging complexity of protein ubiquitination. Biochem Soc Trans 2009; 37:937-953.
10. Skieterska K, Rondou P, Van Craenenbroeck K. Regulation of G protein-coupled receptors by ubiquitination. Int J Mol Sci 2017; 18:923.
11. Mohammed M, Berdasco C, Lazartigues E. Brain angiotensin converting enzyme-2 in central cardiovascular regulation. Clin Sci (Lond) 2020; 134:2535-2547.
12. Mukerjee S, Gao H, Xu J, Sato R, Zsombok A, Lazartigues E. ACE2 and ADAM17 interaction regulates the activity of presympathetic neurons. Hypertension 2019; 74:1181-1191.
13. Ch′ng S, Fu J, Brown R M, McDougall S J, Lawrence A J. The intersection of stress and reward: BNST modulation of aversive and appetitive states. Prog Neuropsychopharmacol Biol Psychiatry 2018; 87:108-125.
14. Doobay M F, Talman L S, Obr T D, Tian X, Davisson R L, Lazartigues E. Differential expression of neuronal ACE2 in transgenic mice with overexpression of the brain renin-angiotensin system. Am J Physiol Regul Integr Comp Physiol 2007; 292: R373-R381.
15. Bhandari D, Robia S L, Marchese A. The E3 ubiquitin ligase atrophin interacting protein 4 binds directly to the chemokine receptor CXCR4 via a novel W W domain-mediated interaction. Mol Biol Cell 2009; 20:1324-1339.
16. Xiao K, Shenoy S K. Beta2-adrenergic receptor lysosomal trafficking is regulated by ubiquitination of lysyl residues in two distinct receptor domains. J Biol Chem 2011; 286:12785-12795.
17. Xu P, Sriramula S, Lazartigues E. ACE2/Ang-(1-7)/Mas pathway in the brain: the axis of good. Am J Physiology Regul Integr Comp Physiol 2011; 300: R804-R817.
18. Yamazaki O, Hirohama D, Ishizawa K, Shibata S. Role of the ubiquitin proteasome system in the regulation of blood pressure: a review. Int J Mol Sci 2020; 21:5358.
19. Nanami M, Pham T D, Kim Y H, Yang B, Sutliff R L, Staub O, Klein J D, Lopez-Cayuqueo K I, Chambrey R, Park A Y, Wang X, Pech V, Verlander J W, Wall S M. The role of intercalated cell Nedd4-2 in B P regulation, ion transport, and transporter expression. J Am Soc Nephrol 2018; 29:1706-1719.
20. Miličić Stanić B, Maddox S, de Souza AMA, Wu X, Mehranfard D, Ji H, Speth R C, Sandberg K. Male bias in ACE2 basic science research: missed opportunity for discovery in the time of COVID-19. Am J Physiol Regul Integr Comp Physiol 2021; 320: R925-R937.
21. Sakakibara M, Uenoyama Y, Minabe S, Watanabe Y, Deura C, Nakamura S, Suzuki G, Maeda K, Tsukamura H. Microarray analysis of perinatal-estrogen-induced changes in gene expression related to brain sexual differentiation in mice. PLOS One 2013:8: e79437.
22. Liang H, Wu X, Chen X, Wang Y, Li Y, Pan B, Li M, Liu H. Gender difference in association of NEDD4L gene variants among southern Han Chinese with essential hypertension—a population-based case-control study. Clin Exp Hypertens 2014; 36:309-314.
23. Faulkner J L, Belin de Chantemèle E J. Female sex, a major risk factor for salt-sensitive hypertension. Curr Hypertens Rep 2020; 22:99.
24. Russo C J, Melista E, Cui J, DeStefano A L, Bakris G L, Manolis A J, Gavras H, Baldwin C T. Association of NEDD4L ubiquitin ligase with essential hypertension. Hypertension 2005; 46:488-491.
25. Sile S, Velez D R, Gillani N B, Alexander C A, George A L Jr, Williams S M. Haplotype diversity in four genes (CLCNKA, CLCNKB, BSND, NEDD4L) involved in renal salt reabsorption. Hum Hered 2008; 65:33-46.
26. Li N, Wang H, Yang J, Zhou L, Hong J, Guo Y, Luo W, Chang J. Genetic variation of NEDD4L is associated with essential hypertension in female Kazakh general population: a case-control study. BMC Med Genet 2009; 10:130.
27. Huo L, Gao Y, Zhang D, Wang S, Han Y, Men H, Yang Z, Qin X, Wang R, Kong D, Bai H, Zhang H, Zhang W, Jia Z. Piezo2 channel in nodose ganglia neurons is essential in controlling hypertension in a pathway regulated directly by Nedd4-2. Pharmacol Res 2021; 164:105391.
28. Xia H, Feng Y, Obr T D, Hickman P J, Lazartigues E. Angiotensin II type 1 receptor-mediated reduction of angiotensin-converting enzyme 2 activity in the brain impairs baroreflex function in hypertensive mice. Hypertension 2009; 53:210-216.
29. Radley J J, Gosselink K L, Sawchenko P E. A discrete GABAergic relay mediates medial prefrontal cortical inhibition of the neuroendocrine stress response. J Neurosci 2009:29: 7330-7340.
30. Nasimi A, Kafami M. Vasopressin and sympathetic system mediate the cardiovascular effects of the angiotensin II in the bed nucleus of the stria terminalis in rat. Neurosci Res 2016; 108:34-39.
31. Thirouin Z S, Figueiredo M, Hleihil M, Gill R, Bosshard G, Mckinney R A, Tyagarajan S K. Trophic factor BDNF inhibits GABAergic signaling by facilitating dendritic enrichment of SUMO E3 ligase PIAS3 and altering gephyrin scaffold. J Biol Chem 2022; 298:101840.
32. Suthakaran N, Wiggins J, Giles A, Opperman K J, Grill B, Dawson-Scully K. O-GlcNAc transferase OGT-1 and the ubiquitin ligase EEL-1 modulate seizure susceptibility in C. elegans. PLOS One 2021; 16: e0260072.
33. Xu Z, Herbert J. Regional suppression by water intake of c-fos expression induced by intraventricular infusions of angiotensin II. Brain Res 1994; 659:157-168.
34. Song K, Allen A M, Paxinos G, Mendelsohn F A. Mapping of angiotensin II receptor subtype heterogeneity in rat brain. J Comp Neurol 1992; 316:467-484.
35. Liénard F, Thornton S N, Martial F P, Mousseau M C, Galaverna O, Meile M J, Nicolaïdis S. Effects of DOCA pretreatment on neuronal sensitivity and cell responsiveness to angiotensin II, in the bed nucleus of the stria terminalis in the rat. Regul Pept 1996; 66:59-63.
36. Wang L, de Kloet A D, Pati D, Hiller H, Smith J A, Pioquinto D J, Ludin J A, Oh S P, Katovich M J, Frazier C J, Raizada M K, Krause E G. Increasing brain angiotensin converting enzyme 2 activity decreases anxiety-like behavior in male mice by activating central Mas receptors. Neuropharmacology 2016; 105:114-123.
37. Lee K Y, Zhu J, Cutia C A, Christian-Hinman C A, Rhodes J S, Tsai N P. Infantile spasms-linked Nedd4-2 mediates hippocampal plasticity and learning via cofilin signaling. EMBO Rep 2021; 22: e52645.
38. Van Huysse J W, Amin M S, Yang B, Leenen F H. Salt-induced hypertension in a mouse model of Liddle syndrome is mediated by epithelial sodium channels in the brain. Hypertension 2012; 60:691-696.
39. Zhang Y, He X, Meng X, Wu X, Tong H, Zhang X, Qu S. Regulation of glutamate transporter trafficking by Nedd4-2 in a Parkinson's disease model. Cell Death Dis 2017; 8: e2574.
40. Xu J, Sriramula S, Lazartigues E. Excessive glutamate stimulation impairs ACE2 activity through ADAM17-mediated shedding in cultured cortical neurons. Cell Mol Neurobiol 2018; 38:1235-1243.

### Example 3

### ACE2 Ubiquitination as a Target for Salt-Sensitive Hypertension

### ACE2 is Part of the Brain Renin-Angiotensin System

In the 1970's, researchers started identifying components of the Renin-Angiotensin System (RAS) in individual tissues, as illustrated in this cartoon, supporting the concept of local renin angiotensin systems See Lavoie, J. L. and Sigmund C.D. Endocrinology 2003; 144:2179-2183. It is now recognized that components of the Renin-Angiotensin System exist throughout the body and contribute to local organ and tissue functions. Our main interest lies in the brain RAS where all the classic components have been identified in the various nuclei involved in the central regulation of blood pressure. Some of these nuclei are involved in baroreflex mechanisms, buffering the rapid changes in blood pressure. This is the case for the NTS, CVLM, RVLM and NA. Other nuclei, located in the hypothalamus, like the PVN, are involved in long term regulation of blood pressure. Within these nuclei, local and blood-borne Ang-II interacts with AT1 receptors to modulate sympathetic and parasympathetic drive, ultimately leading to modifications of cardiac, renal and vascular functions.

ACE2, a homologue of ACE, was discovered a decade ago and is now recognized as a component of the RAS. Our lab previously identified the presence of ACE2 in the mouse brain, notably in regions involved in the central regulation of blood pressure, like the SFO, PVN, RVLM, NTS and the DMNX. Therefore ACE2 appears to be in a position to modulate blood pressure regulation.

Ang-II Promotes ACE2 Translocation from Membrane to Cytoplasm Via Ubiquitination

As described in Deshotels et al. Hypertension 2014; 64 (6): 1368-1375, in control conditions, ACE2-GFP was strongly co-localized with Wheat Germ Agglutinin, a plasma membrane marker showing that in baseline conditions, ACE2 is predominantly localized at the plasma membrane. When treated with 100 nM Ang-II for 4 hours, ACE2 localization at the plasma membrane was diminished as the enzyme displayed accumulation within the cytoplasm. To further analyze the subcellular distribution of ACE2 in presence of elevated Ang-II, N2A cells were co-transfected with Rab7-dsRed, a lysosomal marker. ACE2 did not co-localize with Rab7 in control conditions, but displayed significant co-localization with the lysosomal marker following Ang-II treatment suggesting that ACE2 is internalized from its functional site and subsequently targeted for lysosomal degradation in the presence of Ang-II.

### SUMMARY

Ang-II mediated hypertension is associated with ubiquitination of ACE2. Nedd4-2 is upregulated in hypertension, mostly in males, and contributes to ACE2 ubiquitination. ACE2 is expressed on GABAergic neurons within the BNST which maintains an inhibitory tone over the PVN. Prevention of ACE2 ubiquitination prevents the development of salt-sensitive hypertension.

### EQUIVALENTS

Those skilled in the art will recognize, or be able to ascertain, using no more than routine experimentation, numerous equivalents to the specific substances and procedures described herein. Such equivalents are considered to be within the scope of this invention, and are covered by the following claims.

What is claimed:

1. A recombinant human Angiotensin Converting Enzyme type 2 (ACE2) polypeptide or fragment comprising one or more substitutions selected from the group consisting of K769R, K770R, K771R, K773R, or K787R, wherein the recombinant polypeptide or fragment thereof is resistant to protein degradation.

2. The recombinant polypeptide or fragment of claim 1, wherein the polypeptide comprises an amino acid sequence according to:
MGVIVVGIVILIFTGIRDRRKKNKARSGENPY
ASIDISKGENNPGFQNTDDVQTSF;
MGVIVVGIVILIFTGIRDRKRKNKARSGENPY
ASIDISKGENNPGFQNTDDVQTSF;
MGVIVVGIVILIFTGIRDRKKRNKARSGENPY
ASIDISKGENNPGFQNTDDVQTSF;
MGVIVVGIVILIFTGIRDRKKKNRARSGENPY
ASIDISKGENNPGFQNTDDVQTSF;
MGVIVVGIVILIFTGIRDRKKKNKARSGENPY
ASIDISRGENNPGFQNTDDVQTSF;
MGVIVVGIVILIFTGIRDRRRRNRARSGENPY
ASIDISRGENNPGFQNTDDVQTSF,
or a sequence of at least 90% identity thereto.

3. The recombinant polypeptide of claim 1, wherein the polypeptide consists of the amino acid sequence according to:
MGVIVVGIVILIFTGIRDRRKKNKARSGENPY
ASIDISKGENNPGFQNTDDVQTSF;
MGVIVVGIVILIFTGIRDRKRKNKARSGENPY
ASIDISKGENNPGFQNTDDVQTSF;
MGVIVVGIVILIFTGIRDRKKRNKARSGENPY
ASIDISKGENNPGFQNTDDVQTSF;
MGVIVVGIVILIFTGIRDRKKKNRARSGENPY
ASIDISKGENNPGFQNTDDVQTSF;
MGVIVVGIVILIFTGIRDRKKKNKARSGENPY
ASIDISRGENNPGFQNTDDVQTSF;
MGVIVVGIVILIFTGIRDRRRRNRARSGENPY
ASIDISRGENNPGFQNTDDVQTSF,
or a peptide at least 90% identical thereto.

4. The recombinant polypeptide of any one of claims 1-3, wherein the polypeptide comprises one or more post-translational modifications.

5. The recombinant polypeptide of any one of claims 1-4, wherein the polypeptide exhibits at least one activity selected from the group consisting of resistance to ubiquitination, resistance to angiotensin II-induced degradation, enhanced enzymatic activity toward angiotensin formation, or increased cellular expression levels.

6. The recombinant polypeptide of claim 1, wherein the polypeptide is isolated from or secreted by a host cell.

7. The recombinant polypeptide of claim 6, wherein the host cell is a mammalian cell, an insect cell, a yeast cell, or a bacterial cell.

8. A nucleic acid encoding the recombinant polypeptide or fragment of claim 1.

9. A vector comprising the nucleic acid of claim 8.

10. A cell comprising the nucleic acid of claim 8 or the vector of claim 9.

11. The cell of claim 10, wherein the cell is a mammalian cell, bacterial cell, yeast cell, or an insect cell.

12. A pharmaceutical composition comprising the recombinant polypeptide of claim 1, the nucleic acid of claim 8, or the cell of claim 10, and a pharmaceutically acceptable carrier.

13. The pharmaceutical composition of claim 12, further comprising at least one additional active agent.

14. A method of treating a subject afflicted with a cardiovascular disease, the method comprising administering to the subject the recombinant polypeptide of claim 1, the nucleic acid of claim 8, the cell of claim 10, or the pharmaceutical composition of claim 12.

15. The method of claim 14, wherein the cardiovascular disease comprises hypertension, type 2 diabetes, or heart failure.

16. A method of treating a subject afflicted with a pulmonary disease, the method comprising administering to the subject the recombinant polypeptide of claim 1, the nucleic acid of claim 8, the cell of claim 10, or the pharmaceutical composition of claim 12.

17. The method of claim 16, wherein the pulmonary disease comprises acute respiratory distress syndrome (ARDS) or COVID-19.

18. The method of claim 14 or claim 16, wherein the administering is intracranially.
